SEQUENCE LISTING (68 sequences)

SEQ ID NO: 1 - DNA, 6730 bp, Mus musculus
gagcatgctccacccggtagtagcaggctgggtggctcgtgctggtccccgccgggcgaagcggcagcggctgtggcgcgggacagtatgcgggaaccggtcgtgatgggtcctgccaccgtgatggttgaagcttttgaggtttgatgacgctgcagcatgtatagtgtggaagatctcctgatttctcatggatacaaaccagcccgagatgccgcagcaccatgtgaggacaaatcagagagatgcaggtcgacaaggacaggaccccgagctggccaaggcctattgaatgggtacaaggatggcgctacagcccatacgcacagtaggacatccctggggacaggacacgtgagcaactctgaaaaccgcatcagcaggccaagaggccacagggagcaccaaagcacttctagaactcctgaggcacggtttttaaatcaaccctccttagcatggtcctctcagccccaaagtggtagagacgacatctactggagcagaggaagacaggagggcagtggctccctgtgtcccagggactggaaagaactggaaagcagaggaatggctcaggcttacagtctgcccgtccatgtgagagagaatctgtgggaagttgcaggaaggacagagcatgtgatgaagaatgccatctgggaagaagagctgagaatgcaggacatgagcttggaaagctggaagaagccaagggagttagggaggcaggcatccgatggggatggacggaaaaggccccaggaaaagttcgagggtctgtacccatttgttcatggggagcacacgtctcagaacagaaaaaaatcccagtcattgccccgagctctttctcctaagagcctgaatttcacagaaattcctgttccactacatgatggtcacataacaggtgtcccaaaagtgccaccatatcctcctagcttcccatccccttcggaacccatgaggaaccttgagaaggctagctcctcaggcccttttcccaggcctaagtttgggaaaccccttaagactccgtgttatagctctcactcacagcccaggggagaaggtggatttcaggaccaccagcacagggacccacgtggctcctacccaacaagaagtaaggatcccagccatgagttgggtatgctggatcctggcttggagcccccagtgtatgtgcctccaccttcatataggtcaccccctcagcacattccaaacccataccttgaagatcctgtgcccaggcatgtgagcagcaaccagagtcagcagcaagtacctgagaaacctgagaccagctgtccacttccttctggctctcttgcagctagggatctctatgatgcaatgcctggctctcctccgcaaggtcttcctccacaaccctatcctattgccacccatgggggttctattcagtacattccatttgatgatccacggatccgacatatcaaactagctcagcccccagaattctatgaagaggcaaagcttgatgatacatcctataaccctggtttactcactacccaagagccagccattgggaaaagacagtacgatgatgccccttcggtaccacggggcccaacgccttcaccagtcaatgagcagagctctgcctttgtccattccagtccccggtggctgcagggccagctccccctgggcattggacctggaggcttccatggccaaacagagcatcatgtcatgggcggattaacaactaatgtgacagacatcaaggcagaaggccatgcctcttcaccacagccacagagtgagggtacctgcaaaacctacactaagctcagaaagtttgaaactggggttcagagcaagaaaagttcaaagaagaaaagtaacgcaactatattttgtttggtctccatcccagttaaatctgagtcacttgtgctagctacggatacaaacaacaatgactttaagctggttgctgataagacccgagggctgtgccagggttcagccctgcaggagcagagtctgctgagtatgtcttccactgacctggagctgcaagccctcatgggaagcatggcttggagaagaacatctccaaggcaaggtctgagggagtcagaagatggccaaattgatgaccccagaatcctccatctcatcaaacccaaagaacttcaggcttccagcccttggccaggacaccaatacagagatcaacagacccaaaccagtttccatgaagactccaaaagctcacagctcctccctgccacaaagccaggagaggccagcaatgtagctccgaccccaacatgcccagataccactgcctccgaagtatgcctccatacagccttagcattcagtgatcaaaatcagaagcccagtgtacctcacctccaaggacaaacgtcccttagcccgtctcgaaacagtgctttctcaaggacttcctcagccataaaccaggcatctatgtccaaagggacctctgaccagctccctggtgccaaccctgttcccaagccagaggtggtgaagggggagtccacaacaggccagtgcaatagcacacaactcttcggtcagtttctcttgaaaccagttagccggcggccctgggatttgataagtcagttagaaagttttaacaaggaacttcaggaagaggaagaaagccatggtggtagtggtagtgaggacagtgaggcagaacagccagaggactgtgcagattccagaaccaagtcctgggctctccagggaactagaacagcacagcagcctgcagggctggcgctggagaatgtagcttccccagatagaagacttaatgactcacagagctggaatgaagaaccaaagcctggccactcaagtgtccatccacagtccctgggcccatcacaggaggaaggcagcagaggtgttccagtccaatgggcagatggaagcctgactgcagagcagaaaagccaggaggatttgaatgggatgtgtgagcgagactttagcccaaggcctgtgagcaggattgcacccattgacacaaaagcagcccctttatactgtctgtcagaaccaagaggaagtcaagaactcaccaaattcggcgatgctgtagggtctgtgcagctgggcagagagactcccacacaggtgggtaatggtggggacacagaggtcctaccctgtgtccttctgccactggctgacaaatatcgaggccactcaacaccagactttcggtctttagagctcacactgggacaagaacagaatgcctataaattagagtgtctggatttagagaacaccgtggaagtccttccaagtgagtctctgcaggaaagggcagagaggatcctgggcatagaggtggccgtggagtcccttctgccaagtgccaggagaacagaacaaagccagcttcctgagcctgatgcaagtgcctgcaacccaagttcatccagagaggactcatcacacagcttggcactaccagtggggcccaaagtggccactgacgccttctatggcaggaggaagtgtggctggactgagagtcctctttttgtaggagaaagggccccccaggcttctatatgctcagatgtggatggcttccctacaagccaggccaccagtcctgagcctgggaaaaaggatgaggaggcgaaagcacccttcaagtccactctgttccatttcatggaaaagtccacaaatgtggtgggtcctgaaaagaggctcagaaacccttccaaagtggttgagaacttacaagaaaaactggtatcacccccaaaaaaggcagactctgttcacttgataagaatgagggaggtcaactccttgtctcagatgaggtgtctgagctccaagagcgctgactccgtggaggagcctgaccccttaaaggtcatcaaaagttcagcctggctttcagaaggccttacttccctgggtggtaaagacgaagcctggcaagcagggcacctgccctctgtctctcagaatgaaaacggacaccccgaagtgccgagggacaagatgtcagaccaagacttgtggtgtgcagattcctatgatccaagccgagtggagagggtgtgatggctgctggtggagcccccaccacctgctagtggaaagctctctggttggcccacctgtcttgctcaggcagaagctggaaactgtggggtttgcagtgtcccggctcctcctttgcctgccacccctacagaattgttacgaagataaagcagtgatctgctgacaccatctgtctctatagcatggatatgtaatccctatatttagaacagagtatttacatcaattcccttccaggtggttaggagtgtggcctaggagtttggacactttgagtgtaaggggaatgtgagtgagcgagtgagtaagtgagtgagtgagtgagcgagcgagtgaatgagcgagtgagtgagtgagtgagcgagcgagtgagtaagaatgacccatttttattactaacccttcggcagcttggagttaggtgtgagttaactgctgtgaactcagaatcgcgtcttggtgttcctgaaggctcgcactcgctgtaacttacccactccacttggtttcaaggcaaacgtgctgtgctcgctcagctttgcactcagtcttccctaggttttaccttctgtgtgaatgtttagatattctttatatgaaaaataattgctgttctaaaatagaccatgctaacaaaataactatatttctttttataaaattgctatttttctatggtatataaacagttcttaggtggcagatttttaaggaagtgctattttatttaagagtctatccctatgtgtgtgagggacccaagagggcccaccttggctctaaagcctttgtccctgctccaatgtcagctctgttccttttcactgtgtttcccaatgtcattgaagaccaatcacatgttcattagctctcataaagatatttgcacagacagagctgtgagagtacatttgaagataccgctctcgaaatcaaccaacttcacggtcattttgcatccatgtccactagacttgccaacccccatacttctgcctttgtattcctatggggaatcttccccgagaagagacgtttctcctgcctactgccttgctcctttaaagaaacatatatttttgtctagaatgactagttaatcttgcatattctgaaaagtgcttgggatcagtacctaggtggcattctatgtgcacacataccatgacggaagtataaaggggcccgtgtttccactattgattggcctgcagagttcgtgtattttgtggcatttcacccttctgcctgtgaaaggtttttgttaactaatattttccaaagcctttgtccccttccttcccctcttacctcttcccctgtcctttcctcttttcttcccctcaccctccacctctctttctcgattaattttttgagacatttagacattttctgacatgccaaattcctgcagtatgagcccatggctggtcagtcctctggtcctaagttcaaacaggactttcacagtctcttatctccaccatatcattttatttcctaagaacctcacatgcactgactggactacacaaagcagagtccaagtagtctcgatacaccttcccagatgcacacatctacagtcttgatttgaacctgtttctcatggccgtccttgtccaaagcttggctcacgtagggaggggtttgagctgtaaatctccctggggcagtggacaaacaggtgagtggtttgcaatggtggttgaatcatctgcgaggggccagtgaaggctaaagtatactagtgtctgtgtgcatgtgtatacatgtgaagaccccagtgcacgcctgtgtacctgtgtacatactggtgctgtactgacttcctgtcacataacctgctaccgtctgaagactctaggacagagagaaggatcctgaaggaaagctgttgtgccttatgtatctcagtctggaacaagctgcacttccattttgtcttgtttgagatcgcatcatacttccccacagctggtcagtcacatagtgactattatagattaacttaagttttccatattctagcccataagcaaatcttttggaaggaagtagtaacgttaaacttagaaacttgaaattttgactacaacttttcatattcctaggtaatgtatatctgaccaaaaatgattttgctgaggtcttaaggtcaatacaaaccaccatttcctatgtaaatttcagatgtgcccatgacttttggtggaaaaaaaagtcttccatattctgtgtttgtaattatttttctcattacacatgttggtattcaataatttggagtttgtgaaattaaaaattctaatttcaaccatttgagctaaaaagtataggaggaattgtgcctgtggttgtttcagaacttaaagacatgatagtgaggtggttgtctttggcctcttcgggggaaatttcctgaaggactgaaaatgagatattttacttattctaatgaatatttacacattcagttgtgtttctgtaagccctgggaaacctctaaagatgcaaccagttcctgggagggaccatcaggattatttccataattataaatacatatttcctttattaataaagaccctgattgtgcatc

SEQ ID NO: 2 - DNA, 19 bp, Artificial
  siRNA
cgggaagaggtgagagtaa

SEQ ID NO: 3 - DNA, 19 bp, Artificial
  siRNA
cagcatcacgcgaggataa

SEQ ID NO: 4 - DNA, 19 bp, Artificial
  siRNA
ggatgagagtcctgagctt

SEQ ID NO: 5 - DNA, 20 bp, Artificial
  primer
agagctggagtgaggagctg

SEQ ID NO: 6 - DNA, 20 bp, Artificial
  primer
tccattccgttgctaacctc

SEQ ID NO: 7 - DNA, 20 bp, Artificial
  primer
gacctgagcacatgacaaac

SEQ ID NO: 8 - DNA, 38 bp, Artificial
  primer
tgtaaaacgacggccagttggacaacatgactgctgac

SEQ ID NO: 9 - DNA, 38 bp, Artificial
  primer
aggaaacagctatgaccatcagatggctgtcagagagg

SEQ ID NO: 10 - DNA, 19 bp, Artificial
  primer
atacactgtggtgcggaag

SEQ ID NO: 11 - DNA, 25 bp, Artificial
  morpholino
catgtttgctctgatctgacacgca

SEQ ID NO: 12 - DNA, 19 bp, Artificial
  primer
agagactcctgcttcaacg

SEQ ID NO: 13 - DNA, 20 bp, Artificial
  primer
cacaccagtgcacaaggttc

SEQ ID NO: 14 - DNA, 20 bp, Artificial
  primer
cacgtgaacccatgtgagtc

SEQ ID NO: 15 - DNA, 20 bp, Artificial
  primer
cacagtggaatcctccaggt

SEQ ID NO: 16 - DNA, 20 bp, Artificial
  primer
gcatccgtgtttccaaaagt

SEQ ID NO: 17 - DNA, 20 bp, Artificial
  primer
aagggcctagaccagagagc

SEQ ID NO: 18 - DNA, 20 bp, Artificial
  primer
tcggctctaggttccatgtc

SEQ ID NO: 19 - DNA, 20 bp, Artificial
  primer
agcagatccctgcttctcaa

SEQ ID NO: 20 - DNA, 20 bp, Artificial
  primer
atttgtgattttggccttgc

SEQ ID NO: 21 - DNA, 21 bp, Artificial
  primer
caggctcatgaacttgaaagc

SEQ ID NO: 22 - DNA, 20 bp, Artificial
  primer
agcgatggcctcttctgtaa

SEQ ID NO: 23 - DNA, 20 bp, Artificial
  primer
acacgactccatgttggtca

SEQ ID NO: 24 - DNA, 20 bp, Artificial
  primer
gaagcaccgagagaacaagg

SEQ ID NO: 25 - DNA, 20 bp, Artificial
  primer
ctcggggtagatcctgatga

SEQ ID NO: 26 - DNA, 20 bp, Artificial
  primer
tccactgctgacaaggtttg

SEQ ID NO: 27 - DNA, 20 bp, Artificial
  primer
actggggctccagaggtatt

SEQ ID NO: 28 - DNA, 20 bp, Artificial
  primer
tccatgggtaatccgttcat

SEQ ID NO: 29 - DNA, 20 bp, Artificial
  primer
tcagcaaaaccaaagtgctg

SEQ ID NO: 30 - DNA, 20 bp, Artificial
  primer
ggtctacatcgaccccttca

SEQ ID NO: 31 - DNA, 20 bp, Artificial
  primer
tcttgattgccacacagctc

SEQ ID NO: 32 - DNA, 20 bp, Artificial
  primer
gggcagaatcatcacgaagt

SEQ ID NO: 33 - DNA, 20 bp, Artificial
  primer
atctgcatggtgatgttgga

SEQ ID NO: 34 - DNA, 20 bp, Artificial
  primer
gactcatcagccgtgtctca

SEQ ID NO: 35 - DNA, 20 bp, Artificial
  primer
tggggaacactcacactcaa

SEQ ID NO: 36 - DNA, 20 bp, Artificial
  primer
aatcccatcaccatcttcca

SEQ ID NO: 37 - DNA, 20 bp, Artificial
  primer
tggactccacgacgtactca

SEQ ID NO: 38 - DNA, 20 bp, Artificial
  primer
gagggcagcagtgaagtagg

SEQ ID NO: 39 - DNA, 20 bp, Artificial
  primer
gtgtgtgtccggatgagttg

SEQ ID NO: 40 - DNA, 20 bp, Artificial
  primer
cagccaacactgctgaaaca

SEQ ID NO: 41 - DNA, 22 bp, Artificial
  primer
tcaggagagaaagatgtgattg

SEQ ID NO: 42 - DNA, 18 bp, Artificial
  primer
tgtgattgttgcctcgga

SEQ ID NO: 43 - DNA, 18 bp, Artificial
  primer
taggcaccttctcaccat

SEQ ID NO: 44 - DNA, 18 bp, Artificial
  primer
caaggaggccttgaagtc

SEQ ID NO: 45 - DNA, 18 bp, Artificial
  primer
cacgaccctcttctcctc

SEQ ID NO: 46 - DNA, 18 bp, Artificial
  primer
atccccaacagagacagt

SEQ ID NO: 47 - DNA, 18 bp, Artificial
  primer
aggcctaaacagggtgac

SEQ ID NO: 48 - DNA, 18 bp, Artificial
  primer
gactccccagactggaat

SEQ ID NO: 49 - DNA, 18 bp, Artificial
  primer
cgctcttgtgctgatgtg

SEQ ID NO: 50 - DNA, 20 bp, Artificial
  primer
ctgcgttaagtttctgtgtg

SEQ ID NO: 51 - DNA, 22 bp, Artificial
  primer
ctctctttctcctttacctctc

SEQ ID NO: 52 - DNA, 18 bp, Artificial
  primer
gcagtcttcctgcctcaa

SEQ ID NO: 53 - DNA, 18 bp, Artificial
  primer
atgggttacgcaagttcc

SEQ ID NO: 54 - DNA, 18 bp, Artificial
  primer
tcaaactccaccctgagg

SEQ ID NO: 55 - DNA, 18 bp, Artificial
  primer
gaagacagcctttgcctt

SEQ ID NO: 56 - DNA, 20 bp, Artificial
  primer
gaggacattttcggactcac

SEQ ID NO: 57 - DNA, 18 bp, Artificial
  primer
gactgtcaggaaccaggt

SEQ ID NO: 58 - DNA, 18 bp, Artificial
  primer
tgctctcgaagcccagta

SEQ ID NO: 59 - DNA, 18 bp, Artificial
  primer
tgtgaatgttgctgggtc

SEQ ID NO: 60 - protein, 1320 aa, Mus musculus
MYSVEDLLISHGYKPARDAAAPCEDKSERCRSTRTGPRAGQGLLNGYKDGATAHTHSRTSLGTGHVSNSENRISRPRGHREHQSTSRTPEARFLNQPSLAWSSQPQSGRDDIYWSRGRQEGSGSLCPRDWKELESRGMAQAYSLPVHVRENLWEVAGRTEHVMKNAIWEEELRMQDMSLESWKKPRELGRQASDGDGRKRPQEKFEGLYPFVHGEHTSQNRKKSQSLPRALSPKSLNFTEIPVPLHDGHITGVPKVPPYPPSFPSPSEPMRNLEKASSSGPFPRPKFGKPLKTPCYSSHSQPRGEGGFQDHQHRDPRGSYPTRSKDPSHELGMLDPGLEPPVYVPPPSYRSPPQHIPNPYLEDPVPRHVSSNQSQQQVPEKPETSCPLPSGSLAARDLYDAMPGSPPQGLPPQPYPIATHGGSIQYIPFDDPRIRHIKLAQPPEFYEEAKLDDTSYNPGLLTTQEPAIGKRQYDDAPSVPRGPTPSPVNEQSSAFVHSSPRWLQGQLPLGIGPGGFHGQTEHHVMGGLTTNVTDIKAEGHASSPQPQSEGTCKTYTKLRKFETGVQSKKSSKKKSNATIFCLVSIPVKSESLVLATDTNNNDFKLVADKTRGLCQGSALQEQSLLSMSSTDLELQALMGSMAWRRTSPRQGLRESEDGQIDDPRILHLIKPKELQASSPWPGHQYRDQQTQTSFHEDSKSSQLLPATKPGEASNVAPTPTCPDTTASEVCLHTALAFSDQNQKPSVPHLQGQTSLSPSRNSAFSRTSSAINQASMSKGTSDQLPGANPVPKPEVVKGESTTGQCNSTQLFGQFLLKPVSRRPWDLISQLESFNKELQEEEESHGGSGSEDSEAEQPEDCADSRTKSWALQGTRTAQQPAGLALENVASPDRRLNDSQSWNEEPKPGHSSVHPQSLGPSQEEGSRGVPVQWADGSLTAEQKSQEDLNGMCERDFSPRPVSRIAPIDTKAAPLYCLSEPRGSQELTKFGDAVGSVQLGRETPTQVGNGGDTEVLPCVLLPLADKYRGHSTPDFRSLELTLGQEQNAYKLECLDLENTVEVLPSESLQERAERILGIEVAVESLLPSARRTEQSQLPEPDASACNPSSSREDSSHSLALPVGPKVATDAFYGRRKCGWTESPLFVGERAPQASICSDVDGFPTSQATSPEPGKKDEEAKAPFKSTLFHFMEKSTNVVGPEKRLRNPSKVVENLQEKLVSPPKKADSVHLIRMREVNSLSQMRCLSSKSADSVEEPDPLKVIKSSAWLSEGLTSLGGKDEAWQAGHLPSVSQNENGHPEVPRDKMSDQDLWCADSYDPSRVERV

SEQ ID NO: 61 - DNA, 5636 bp, Mus musculus
ctccacagaaagaagatactcccgtggatggggctaccgaggagccggggtttgagggggaagtccaggagcatggtacagagcagacaggaactgagggggacctggaagctccagatgaagaggcaccaagtagagacagtgaggaaggcatggtccacgctctggaagatgaagactgtgatcacgatccagagacggatgggaccccaacatcgccagatgaaggggcaccaagcagagacagtgaggaaggtgaggaggactgtgatcagggcccaggtatggaggagcatcccatgagtgaagaggagggagaagaggaggaggtgaaggagcacgtgtacaacagtgataacagggcaccctgggatggagaggagcccttccccaatgaggtcattctcacacatgtccgctctcagtcccctgaagttccctgttgggagccaggccctcctgagactcctggagaggcagaagaggattgtgaagacatctgtaacaacacagaacctgggaaacccaatcaggacactggtcaggacacagaggatgccggcatgggatcccctgagagtgaggtgtccccagatgtccaggagcaagaggcagcaacggacaaccctgaggtctttgaggaggactctgcagatgctgcagaaggtgaggatcagatagagcaggaggaaccacccaattgtgacgaggaagcctataatagagatgccgcagcagccaccatgcaggtgggagaggacctcggagaggagggagaccatgtgcaggaggaccctgctgaggaaagctgccagatcattccctttgagagcgacagtgtggaggaggatttctcacctacactcacagagaatccctatgagattttcccaaccgagagcacttccttctgcaataacacctattcccttgacgagtcagccaatgggcacgagccagtgtgcgagatctgtgtagaggaggttcctggtgttggccctccacttaaccagcatgattccctgccagatggatctggagaggactccccggtggtccctgatgtggtggtcgtgccagagaatgaggggcccgtggatgatgcactcagcagtccatacgtgatgggagttggcttgctgagccttggagagggagcgcagtcagacacccaggctgcatcaggcactctgagtgggtacagtacatgggaggaaggggactctgagggagggcaggtcccagtggataggaagaatattgccacaagggcccggcctcactctgggaaggtggctggtcatgttccagaaactgttctagaagaaacgggaccagaaacctgttcatcaggcatgggcatcagagataccagtgatgaagtgaggaagataggtatattgccagagggaaagcctcccgagtgtgttcgggccttgccggccaagcccagagcatttactctctacccaaggtccttctctgtagaaggccgggagagtcccctgtccatgttccgggagccagagggagccgggctggacagccaccgtgtaaggaggaaagaggacaacctctctctgccgggcgccatcggctcctccggtagcttctcacagcgcagccacctgccttccagtggcacctccacaccatcctctgtggttgacatcccacccccttttgacttggcctgcatcacgaagaaacccatcactaaaagctcaccctcactcctgatagacggagacaccctggaaaaagcctctaagaagaagaagtcctccttcaaacgcttcctggagctgacgttcaggaagaagacagagagcaaggtgcacgtggacatgaacctgtcgtcttccaggtcttcctctgagtccagctaccatggtccagccagggtactggaacttgaccgcagaagcctcagcaactcgccccagctcaagtgtcgcactggaaagctccgggcctctgactccccggccgccctcatcttctacagggacagcaagaggaaaggcgtccccttcagcaggacggtgtccagagtggagtccttcgaagaccgctcccggccgccctttctgcctctgcccctcaccaagccacggtccatctcattccccaatgccgacacttcggactatgagaacattccagccatgaactcagactatgagaatatccagatcccccctcgcaggccggtgaggactggcactttcacaaagctgttcgaagaacagagccgagccctgtccaccgcaaatgaaaatgacggctacgtggacatgagcagcttcaatgccttcgagagcaagcagcagagttcagagcaggaagctgagagcgcctacactgagccctacaaggtctgtcccatctcagcggctcccagagaggacctcacatcagacgaagaacaaggaagctccgaggaggaggacagtgcttcaagagaccccagcctctcacacaagggtgaaggacagtctagagcccttgtcatcgctcaggagctgctgtcttcagagaaagcatatgtgcagatgttgcagcacttaagcctggatttccatggagccgtcctgagggccttggagaacgtagagcaagagggcagagagccactggcccaggaggagctgcggcagggcctgcgggagctcccagctatctgtgaccttcaccagggcatcctggagagcctggagcagaggctgggggattgtggggagggccagccgcaggtggccgacatcttcctggcccaggaacaggagttcgagcatcatgccgcacacatcctgcaatttgacaggtacctggggctgctcgctgagagctgcctgctctcaccccggctagccaccacggtccgggagtttgagcagagttcacaagggggtggccagagcatgaagcatcgtatgctgcgtgtggtccagcgcctcttccagtaccaagtgctgctcactgattatttaaataacctgtgcccggactcagcagagtacgacaacactcagagtgctctgactctcatctctaaagtgacagaccgtgccaacgaaagcatggaacagggggaaaacctgcagaagctggtccacatcgagtacagtgtacgaggccaaggggacctcctccagccaggaagggagttcctgaaggaagggacactgatgagggtgagagggaaaagccgacacccccgccacctgttcctgatgaatgacacactcctgtacacacatccccagaaggatgggaagtaccggctgaagagctcgctgccagtggccaacatgaaggtcagccgccctgtgatggacaaagtgccctatgctctgaagatcgaaactcctgagtcttgtctgacactgtctgcaagctcctgcgcggagcgggacgagtggcactactgtctgagcagagcccttccggaggattacaagactcaggccctggctgccttccaccacagtgtggagatccgggaaaggctggggatcagcctcggtgagaggctccccaccctggtgcctgtcactcatgccatgatgtgcatgaactgcggctgtgacttctccctcaccgtgaggcgccaccactgccatgcctgcggcaagattgtgtgccggaactgctctcgaaacaagtacccattgaagtgcctcaagaacaggatggccaaggtctgtgatggctgcttccgggagctgaagttgaggaatgggcctgtcccaggctccatgagagagcgtccagtcagcatgagcttcccactgtcctcgtcccgcttttcctcgggcagcgccttgtcctctgtcttccagagtattagcccctcaactttcaagaagcagaaaaaagtcccttcggctctgtccgaggtggccgcgtcaggagagggctctgccatcagcggctacttgagccgctgtaagagtggcaagcggcgctggaagaaactctggctcgtcatcaagggcaaagtactctacacctacttggccagcgaggacaaagtggccatggagagcatacctctgctgggttttactattgccccagaaaaggaggagggcagcagcgaagtgggccctgtttttcatctttatcacaagaaaaccctgttttacagcttcaaagcagaggacagcaattctgctcagagatggatggaggccatggaagatgccagtgtgttatagcagtcaccaggcccatggactaacaacaaactcctacattaactcgagaacccttccggaagtgactcctgctttcagctcatctggatgcacgtgtgggttctgtgtggagcttgacctcaaggcgaaaccccaagagctatttatagacatcttcttttctgcctccatgtttcccaaccccaatcacagactgtacacccttatctaaaagtgagttaaaatttagtaccttgaagacgtggaaaccaagtgaaaaagacacttagaaatgtgggctttttagtagaaactggctttttctggttttactcctggtgactaagaccactttcaactttccgcactctccatatggtgcctcaggtgatgtgggagccagacaggaagtatcagctgtgtatctaagcactttactatcctgctgagctgcagcctccagggctgtgtcttccaaggaatggctgcaaacttcggtgcaaagtgactcctgcagccttactgtggcttgccaccctaaggagggaggggctcagcaccacaccagggctgccctctaaagcatcacagatccctcaaacatggaagacctgcatctgcgttaagtttctgtgtgcattcctgtgtccacactgtgcaggctgaccatcctgttgatgggagtctgtcctctttgtaacacgtgtccttgggctgtaccagccacaccaagcttactttttgagagaggtaaaggagaaagagagaagttgggtcatcagaaagtggtctgcacagaaggcttggccagcccggaggcactgagtggctggaagacactttgcacacctgaggccgacagcatcacctccctctgggcacctgaggcagagccatcacctgctggccagttctttatgccgaatggattttacaaaatgcaggaggaggaacaggattcctcttcactatccctacagttgggaaaaaaaaaaaaatgcctccattttctgaaatctcttcctgccttgtcccctacaaaaaagatttttgtaaaacaaacaaacaaacaaaccaccatctcaaggacttatttacattaattatgttcagggatttctttttctagtttgaagttctttttcaatgtttttgcagtcggtgtatagttagtaattcaagtcggatggaagacaagtgttactgttcacttcacaatcctaggatccttcctagagtcactcacatgggacccggacaccgagacccaggaacctttagagagtggagctggactcaaggacagtaatcctcttcctttggtttcatttttctatgatttcaattgcaggagaccaactgtggactgaaaatattaaatggaaaattccagaaataaaagtgcatgaatttccagtcaaaaaaaaaaaaaaa

SEQ ID NO: 62 - protein, 1359 aa, Homo sapiens
MYSVEDLLISHGYKLSRDPPASREDNPKGRQAARTGTRAGQGLQNGHEDGPAALAHRKTSAGKGHVSDSESRRSTPRGHGEPQSTSASRTSEAGFCNQPPSAWSSHPPTGNDQAYRRRGRQEARSQKPREHENLEARGMAQAHSLPVHVREGPWEVGGRSEHVMKKPVWEEELRMSGPAKWQNVSLESWNQPRKLGRQMSDGDGERLFQDLYPFIQGEHVLNSQNKGKSRSLPRVLSPESLSCTEIPIPLNERHSPKMPPYPPTCAPNLDSTRNSEKSGCSAPFPRPKFGRPLKPPSYSSHQQSRGGADSSDSQDSQQMDAYVPRHELCLSDPGLEPPVYVPPPSYRSPPQNIPNPYLEDTVPINVCGGHSQQQSPTEKAGASGQPPSGPPGTGNEYGVSPRLPQGLPAHPRPVTAYDGFVQYIPFDDPRLRHFKLAQPQGFCEDIKLDDKSYNSSPVTAQEPAHGGMQPDGAIWNPQSLIPPSGDERGLVLADSSPRWLWGQPPGDGENSGLPNQRDRCVARGQWPDVRGSQHGHTGRQVSSPYSQGESTCETQTKLKKFQTGTRTKKSSKKKMNETIFCLVSIPVKSESHLPDRDMDNNDLKPSADQKNGSDKSPALQEQSLLSMSSTDLELQALTGSMGGRTEFQKQDLGEPEEDRQTNDLSFIHLTKHRELKHSGSWPGHRYRDQQTQTSFSEEPQSSQLLPGAKLGGPSRAALSPKCSDPAASEAQTHTAFPTGDHKQRPSARNLKGHRSLSPSSNSAFSRTSLSVDQAPTPKAGRSQPCVDVHGLGAHPGPKREVVKGEPTGPCNSKQLFGQFLLKPVSRRPWDLISQLESFNKELQEEEESSSSSSSSSSSSEESEAEPQQENRAHCRQEDVGFRGNSPEMRVEPQPRMWVPESPVCRSGRGESKSESWSEELQPGHPRAWPPSPGRFRVEEGGGAPFCSADGSTSAEKRHLEVSNGMDELAGSPFPVTRMSSRSSDAKPLPASYPAEPREPQESPKITSAFSSVKPSEAVPRKFDSGGERGAGLPLSLSNKNRGLSAPDLRSVGLTPGQEQGASELEGSLGEASTIEIPPGESLQARAARILGIEVAVESLLPGIRRAGQNQPAEPDASACTPESPQEELLSRPAPADVPRVSTDAFYGRRKCGWTKSPLFVGDRDSARRAPQAFEHSDVDGVVTSTDPVPEPEPSPLESKFFEQKDVETKPPFRSTLFHFVERTPSVAGSEKRLRSPSKVIESLQEKLASPPRRADPDRLMRMKEVSSVSRMRVLSFRNADSQEDAEELKATTRGQAGLPGGLVSPGSGDRAQRLGHSLSVSKDSISREEKEHPAAQKEKSMDQDFWCPDSYDPSRVERV

SEQ ID NO: 63 - DNA, 1728 bp, Mus musculus
gtctaggagcctcgcagcctcacgtccatgtccgtggcggtggcgcctgtggctgtgcaccccgacagcatgctctccgaagcggaggagcccagggaagtggctacagatgtcttcaattccaaaaacctggccgttcaggcacaaaagaagatcctgggcaaaatggtatccaaatccatcgccaccacgctgatcgacgacaccagcagcgaggtgctagatgagctgtacagggtgaccaaggagtacacccagaacaagaaggaggcggagagggtcatcaagaacctcatcaagacggtcatcaagctggccgtcctccacaggaacaatcagttcaatcaagacgagctggcgctcatggagaagttcaagaagaaggtgcaccagcttgccatgacggtcgtcagcttccaccaggtagagtacaccttcgaccgcaatgtgctgtccaggctgctgaacgagtgccgagagctcctacacgagatcattcagcgccaccttaccgccaagtctcacggacgggttaataatgtctttgaccatttttcagattgtgattttttggctgccttgtacaatccctttggaaagtttaaacctcacttacagaaactttgcgacggcatcaacaaaatgttggatgaagagaacatatgagcttgcgaagtaagattgtgaccagcagcgagttaggtgaagtccagcaccgatgacttgtaaagggaagacggagactcttttatcaatcacatgtatttcagaaaggcttggcccccatatgacctgttattgttgttggacattaatagtagtacttctgtgtggcttgttgtatttgaaaggaaaaaaaaaagtgtattgccaaaaattctggctgcaaacgtcttaatgattggcaagatgtgagaaaaacggatagttggtcattcagatgtcaagggatacaaagacagatgagtgtggccccaagcgctggcacttcttgtgttttagggaaatcatgttggtggcacgttggatatttctaatatgtataaagccgtgtatcttgactcaccttgatcctttgctacgtctgtgtatctctcttacaatgtcgagaccctctctttgctgtcattgatccttttgaaacaactctgcttctttggttacagatcgtcattgaccttcaggagttaaatttgaggcagtcaaaccgaatgctcaggaaagcaaaaaaaactgtgcatctgaaatcatgactgtgggagtgaacaatcttagacatcatgagcagtgtccattgcaagagtgtgtttttgaagctgacttctaccttttagtactatcagtgatacttagagtctcagagctagtggcatgatattgccttgaaatatctgcttagggcataactttgatgtcatcttgaacatgtactgacatgttcccgtattaacatctatttcacctgtaggaagaataaatctctagattggctttgatattgagacaataaaatgtaagtagcgtttcaaaaagaaaaacttcttgaatttaaagatgaattcatcagactgatttatgggtgaaaggatagaaaggacgttgctaagtaacataagaaagatgcatgtatttcagtgtgtaacagggattgatgaataaaagatgccattttttatttaaaaaaaaaaaaaaaaaaaaaaaaaaaaaaaaaaaaaaaaaaaaaaaaaa

SEQ ID NO: 64 - DNA, 2365 bp, Mus musculus
tttctcgggggcgtggcctcctagccagcggccgggctcagatccagcggccaatgcctctggccccacagaggcacctacagaaaactgaaggcttgacacatcccgaggagcagaacccatggacaccttcagcacgaagagcctggccctgcaggcccagaagaaagtcctcagcaagatggcttccaaggccatggtggctgtgtttgtggacaataccagcagtgaggtcttggatgaactgtaccaggccacgaaggagttcacgcgcagccggaaggaggcacagagggtagtgaagaacctggtgaaggtggctgtgaagctggctgtgttgctgagggcggaccagctggacagcaatgagctggcccagctgcagcggttccggggccgcgtccgcagcctggccatgacagccctcagcttccaccaggtagacttcacctttgaccggcgtgtgctggccactgggctgctggagtgcagggacctgttgcaccaggctattggcccgcacctcactgccaagtcccacggccgcatcaatcacatcttcagtcactttgccaatggtgacttcctggccgcgctgtacagcccagcagagccctaccggagccatctgtgccgcatctgtgatggcctcgggaggatgttggacgagggtggcatctgacctggagtcagccacctggagactatcctccactggtgacggtccccaagaactcagaactgcctgtgcctggggtagtctctcctctgtttctcctgctgtaacttatctgcttttgtctagcttctgcttcgtctcctgacctcctcttcttacttctgagacaggtctcatgtagcccaggctgaccccaaacaccctatgtagcaaaggctgaccttgacctcctgagcctcctgcccccaccaccgagagtgctggatgataggtgtgtgccaacgccccactgtctggacattctcagtctttcaaataaggttgtggagaattgggagacaagaattgtactcccctgacccttacccctgccaaatgactatgtttgtttcctaagcctcaggtcgtcccaaggtgacttgctttagaaaatggagttcaaactcttgttgtaccagactagagttgaacaagtactggactgctacaacaggtccttatgtatagattggcccaggaaggctcactctgccaatgctgcaggacactggacactgaggagtgatgctactgcaaaccaagaacaccagcagacagatgctgtgcggcagatcgtggcttctctcaatggttttgaaaggagagactccaagcacacttcggtgttgggatctgaggtcctgttaggggactggaccctcgcattgaagagaagaaataaatagagccggtgagatggctcagcatgtaagaatgcttgtcaccaaacttgtctggagtttaccaaaatctgtgtggtggatggagtcttagaaagctggcctctgacctccatgtggatgtgcccacacagaaccacaccgcggcttccggtgcttggaaacatggcagggccctgaccactcagcaaggaggcgctaatcaggcagaaatgtggggtcttcactgagtgatagtacaggacttgctgtgaaacatggggaccccagaggctgtccccagcttcggaccagggtaaagccactggagacacgagcatgtgttacattataattccatatttgggaaacaaatccatcttttctctttgaatggtaccgagttggtaactgacactgaccacgcatgtgaaagcctgcagcctctactatctgagtgtgcctgtggtcatgtgagacaccgttaactggcttaacccaactggtacttagatcttttatccttggaaagcagacttcagctacacatgtgtctagctgaggtgtgggttagttgtctagaatcctccagcgagggttcggggggcatggtcaggggtggagcccagcctagaatcctccagtgagggactgggggcgtggccagtgatagatgtacctagaatccccaggtcagaagcttggctcaggggtagcatgcatgtcatggggtcgacccctgagatctgtgaagatcgagtctgtggaggggctggcacagctgtggaggggctggcacagcggttacgcagtgctgccgatggaaaggctatctggggcattgggagaggacacaaaatgtgcagagctgagtgtatttaaggcaatgtgtacaacctggagcctgtggtgacgggtgggagtcactgcctcaatgtttcagtgtatgcagacagatgcaagtacaaagaagagtctgtcaatagct

SEQ ID NO: 65 - DNA, 3573 bp, Mus musculus
ggagacagagactctggagggtgcaaggaagtgtctttagctgagctgggctgggctgggacgggaaggtagactgaagctgcctgaactgctcatcccgctgggtctgagctcccagtggggcggttcgcatgcctggaaggactctaagcaccctcagacctcttactctgaggttcaagccaatccttcagggtgcttgctgagttggtgacccatctctggaactggtgtccctgggactcactgcccagcttcacttcattcagcaccatggaagatgatggttacaactactatggggctgacaaccagtctgaatgcgactacgcagactggaagccctctggagcgctcattcctgccatctacatgttggtttttcttctaggcaccacaggcaatggcctggtgctctggaccgtgtttcgaaccagccgcgaaaagagacgctcagctgacatcttcattgccagcctggcagtggctgacttgacctttgtggtgactttgccactgtgggccacttatacctaccgggagtttgactggccttttggaaccttctcttgcaagctcagcagctacctcatctttgtcaacatgtacgccagtgtcttttgcctcaccggcctcagctttgaccgatacctggccattgtcaggccggtggccaatgctcggctaaggctgcgagtcagcggggccgtggccacagcagtcttatgggtgctggctgcccttctagctgtgcctgtcatggtgttccgttccacagacgcctcggaaaatggcaccaagatccagtgctacatggactactctatggtggccacttcaaactcagagtgggcctgggaggtgggccttggggtgtcctccactgccgtgggctttgtggtgcccttcaccatcatgctgacatgttacttcttcattgcccaaaccatcgctggccatttccgaaaggagcgcattgagggcctgcggaagaggcgccggctgctcagcattatcgtggtgcttgtagtgacctttgccctgtgctggatgccttaccacctggtgaagactctctacatgctgggcagtttgctgcactggccctgtgactttgacatcttcctcatgaatgtctttccgtactgcacctgcatcagttatgtcaacagctgcctcaacccctttctctatgccttctttgacccccgatttcgccaagcctgcacctccatgctctgctgtgatcagagcgggtgcaaaggcacccctcacagcagtagtgctgagaagtcagccagttattcttccgggcacagccagggccctggccccaacatgggaaagggaggagagcagatgcatgagaaatcgattccctatagtcaagaaacccttgtggactagggctgggaccagagggaagcctggtgcagcgggtctgacccaaactttatcctcacgctctgagaatcaggtagcatggctgtactttctcccggattcctcgctccgttctcttgctcatctccgagttctgccttgcccctctcagatgtttgtggtttagcggaaagggatggagccttgcaaacagctcagctgttcaacatttctgaggttgagcttgcacaagtctcttaacttccttaaacttcagtttctttgtttataataaatgagggaaggttatatggaccctaaaatgttgaagtatgagtcctgatctaatttgcttcattctgccattttttccctcagctctctagcacccctttttgctttctctgctggagaaattatttctgtcaactactcacttgctagatctaactctttctcttctagtgggtcccttcccctcaaaccttcctgtctgtaccccagcgccttcttccctgctggactcttaagggttaagtaccctgaaacttgaagagtaacttatcctttgagttcttgtcctgttttctgagaaccagcaggtggctgtatgtaggcaaagaattagtgctctagtccccagcgccaggagctctgtatctttcaaaaaagttggatcagttcattgttggtccaggcaccagggctagatgaatttttgaatagcaatttctctttggcagaatctctgtgatgcttagtgaatcctaccaataacccagtatccttcccatcattttttttttttaaaactgtctgtgagaatctcaaggcaacaggactgaagaagctgtggattgtcctgtccctctgaaggggggagttgggaagggacaacagagaaaggattagaggggagtctgtatttagtttgtatgctgcttgcctcccctatcccaatcctcatccctccacctcctacagaactgtgaactgtagagctgatctttctcctgtctcctcccccaccccaccatctctataggttttggtgctacttggcaccaagagtcactcacttggttaggtaaggctatttgctttttgtctatatgggtgtatataaaggtatatcaatgtgcacatgcatgtgtgtgtacacacacacatacacaagtatcacataaactttatcgatgaaggactagggtgaaccccccactcactttcccctgtgcctctgtaggtctcagagtccacttggggaagaatggacctatggaggtagaatagatccattgtggatccacccccactggatttggggaactccgacggctggtccgtatctaggaaagaagtctatgtttgtgctaggatttctgtagggagctgttttctcagtggcatctggtcctaaggctggagaagtctggagctgggccccagttcctctcccagttccacagtagcaagaagagacttacagactatatatagcagggcccttctcctctgtccctccctgcctctgacttttttctcacccctctcactctctttaggggtcttgaggaggccactttcttgccacaggatgatgaaaaaagggtgagcttttgtgatcctctccatcaggactgagcctcctggatcctctttctcctttaggggggagtaagtttgggaaagagtgactggagcctctgtggggatctccaggacaaaatcaatagtggggcctttgtgagctggacatgggtgtgcatattccgggttttgttcttggagggctctggacaccaaggacaagacatattccatggaaacaggaagagcccagcttgatgcggttggaccatctctggcagagatcctgctcctggagtaggaaagggggttgtggtggtgggggatggagaaagcagctcctcttagctgagctctgcatatctcaggccagaaactttcacacagctactttctccgccaggccattctcaaagcagccagttcgagctgcaggaggcttctgggagacaacaaactcaaccacagcatgggacagatagcagcagaggcagcttttcctcacccccaccccttcttcaggatgcgcttttttttttttaaatgatcttgttactcactgtggttcatcaaataaaactgttttgtgtaaaaaaaaaa

SEQ ID NO: 66 - DNA, 3148 bp, Mus musculus
gaagcattctctctaacagcctggaccaacgggtagttgggagtcgcgggcagagcagcgtcagccacttagagagtttttgccgccgacccgaagccaccaaggccagcttcgcggcgctgccccgcggcggcagagaaggctgcaccaagagcagaggcagcgagcaggagtggggcaggcagccagcggtgcggctggggcgctcaccctcccgcggtccgggagccacgcgagctccgtgcccgcacgcgccagccccggctcgcgcctttctttgcgtccgggtgccctgcctctccgcccactcgccggctcctctgggctgccgcggaccgagttgcagcatgaatctgaggctctgcgtgcaggcgctgctgctgctctggctctccttgactgcagtttgtggagtgccactgatgttgcctccagatggaacaggactagaagaaggaagcatgcgctacctggtgaagcccagaacttcgaggactggaccaggagcctggcagggaggcaggaggaaatttcgcagacagcgcccccggctctcccataagggccccatgcctttctaaagcaggattgaagggctcgccaagtgccctcccggtgccggtctctctactccacagatgaattctcttctctggaaccctcacatctatttggctttcatcttgcacctgttctagctgctgatggtcccggctcttctcacccaccaagttcctctaatggcgtggtctcgcccctcacttggatgatttcattcctctcacaaggatccctttggcccatcctcctggccactctttggactgcccttggagacccagcttctccttgacctgttccatgcggtagtccctatctttcagccgtagccttctcttggctcttccctcttttcatttgccttgggcactggacaagaacactcccttctccagtggattccagcacatccatgctttcttatcaattggacctcttgcctggaagggagcgccgagatgtgcaatgagacttcggttgagaggggttgggtgttggggtccagataaggtgcactggggaaactgaatggtgggcgtgaggaaggggtgtggcgaaatagctgccacagcctcttccttagctctctctggccctgttaaaggaagacgcaaaggaaaggagcccatgaagttctatgtcaccgtgccttctaatgtcttcgctctctctgttctattgcagcttgttcagtccccaggtcagaaggggagagtgtggatcctgacatggttctatactcttctgggggtaattttgccaaaaaaataatcccaaccaatcaaccaaccaaccaaataacccacagaaagtcagagcccaaaggacccttgaagatcacttagctcccttatgcaaaagcttgcacaaataggccctggagagggaagtaacctgcttagagccactgcagagccccacagggcaccagggccctcaactcccactttatgatgccctttttcctcttgatgatgtcctttcaaagcaaatgaagtgccttttgctgggtttagtgaagcgggtggctaggaaggaagagcagggagagacaagccagctgtgaactgctttgtgtgagaatggagcatctccctcccaacgtggcccggaatagtccctaagtggtgaaatgtaccccacacaggcctattcccaggctcatgccattttaattggggttctcaggcactgaaactaccatttagcccctgacactggttgtcatgaacccctgtgttgactgcgcccttgatcttggggcttttgggggatggagaaaggcaaagaaaggaaggagaggtggaagaaggaaggagagatgggaggatgtgtaggtgggatctatcactccaccaagggggtctcatctctcaccaggagccttttgtagcccttccctgaagggagaccctcatcttctctgcctgagcacatgttcttctcagaggcctctcaggagggagaaaagagaagacagaagctcgatattcacttatatatgtgtagggtagggggagtcaggacccccaagccccataataacctgattgcctattcatcctttcccaaagccttcctatctgctgctgctaatgctgctgctgctgcctctgctgccgtttcaaggcccaccctggggagcagggccaggcatagcgtcctcacctcttgggctagaagtgggagctggctggcgcctcttctactgctggtacccctaccagggaccgggccttctccgtctttgtcgtttctgcacctcctgccttatcagcctgccatggccagccccacagggaaagggaggggggaatattggctgagaaagaggagagatagaagcacaagaggggagtaaacgggcccagtgggctgtcttatttaaagtggttatgtgtatttcttatactaatttatatagagatattaaggccctttgagttagagaaactgtccccttcccataagtgtgttcgctatgtttgtaaaaattgttccatgtaaatatgtctttataataaagagttaaaagttgacagtttgcccttactctttgaggtcatattcaggagggcattctttttctcctagggccaatcttgtccctgtgcccacagattgcacgtgtagggaggtgagtgcttgtatcccaaattggttctaggtcaactggcctcaaactgatttgccacaagctcacaaaaagactcgctatgcttaagggccagttcacataaatttgagccccttttaagtcctttggcatctgttcgaatgtcctcatttttctggcagtgtcacttaaaggattttttttccagaagcaattcatttaaggacacgctgatcaaaggtgggatttgtactaaacgctgttttgtagccctggtgggtgccttctgttttatctgcttttgaatactttcgggactttttagccagtttgcctttcttgacaaatgttgtcctcagccataaatacatttggtaatgactttgtatgtatcgttttattgtttcacaaagtggagttgcttgatgaatgagataacctggaaaataaacttcgaggagttcgatagga

SEQ ID NO: 67 - DNA, 7995 bp, Mus musculus
gagctgacggatagcttttaaagcggctgctcacccaccctgtcctgcctccttcctcatgcctgcctcttggagttcactcgctgcctgccgctgctgcaatcactgtccccacactacttttgtctcagacagcagacctgcctgctatggcagagccccgaaccctccttctgctctgtgtcctggtcctctgcctgtcagactccagcttcatccgagggcagacggtacggtctaaacgttgcgacatccacaccaagtttgtcactcacacaccctgtacggcgtgtgcagctatcaggaggcaactatgtccctggggctggtctcggaatttcccagagaagatacttctggactgccgctatgagttgcagctcaggggcgccgcgatatccttaagtggctgcagccaggaatgctggaaggatgtggttcagaaggcctgctgccccggctactggggatcccagtgctttgagtgccctggtggtccggcgacaccgtgtagtggccacgggacctgcctggatggcatagaaggaaacgggacctgtgtgtgccaggaaaacttcagcggttcagtctgccaggagtgccgagaccccaacagatttgggcctgactgccaatcagtgtgcaactgcgtacatggcgtgtgtagccatgggccacggggagatggaagctgccggtgttttgctgggtatactggcccccactgtgaccaagagctcccagtctgccagagtctgaaatgcccccagaattcccagtgctctgcagaggcacccacctgcaagtgcctgcccggatacactcagcaagacaatgtttgtcttgctcctgacccctgccagccatctgcttgctccccactggcccgatgctcagtgactccccaggggcaggctcagtgccagtgcccagagaattaccacggagatgggaaagtgtgtctgccccgggacccttgcctcaccaactttggtggctgccccagcaactctaccttctgcctgtaccgtgggccaggcaaggccacctgtatgtgccgaccaggtatgacaagcattaacaacaacgcttctgaggggtgccatgtctcctgcaaaccacattcctgcgacagatcagctacctgtcaggtgacccctgacagaaagaccagctgcgtgtgcaaaaacgacgaggtgggtgacggtcatgcctgctatggacatctactccatgaggttcggagggccaatcagaatggcctggtgttcctgcggttaagagcagccattgccatgctggagcagggctgccaggagattcttaccacgtcggggccattcaccgttctggtgccatccatgttctctgtctcctctgtctccagtaacatgaatgcgacgctggctcagcagctctgccgacagcatgtcatcgcaggggagcacatgctggagaatgcagggcccccaagcacacgcaggtggtggacgctggctggccaggaggtcaccatcactttcaaaaacatgagatatgcctacaagtacgaagatcagccccagcagttcagcatccacaaggccaactacatcgcagctaatggggtcttccatacggtcactgctctacgatggcagcttccacccccacttccaggggactccaagaaaactgttggccagatacttgcttctacagaagttttcacacggtttgaaacgatcctggagaactgtgggctgccatctatcctggatgggcctgggcccttcacagtctttgccccgagcaacgaagctgtggacagcctgcgagatggccgactgatctaccttttcacagcaggcctttccaaacttcaggagctggtacggtaccacatctacaaccatggtcagctgacagttgagaagctcatctctaaaggacgtgtcctcaccatggccaaccaggtcctgactgtgaatatctctgaggagggacgcatcctgctgggaccggagggcatccctgtgcggagggtcgatgtgccggctgccaacggtgtgatccacatgctggaaggcatcctgctgccccccaccattttgcctatcttacctaagcactgtgatgaagagcagcaccagactgtgctgggttcttgtgtggattgccaagccttgaacaccagtgtatgccccccaaacagcgtgaaaatggatatcttccccaaggagtgcgtttatatccacgacccaaatgggctcaacgttctgaagaaaggctgtgctgactactgcaaccagaccatcacaaaacgcggttgctgcaaaggattttttgggcctgactgcacacagtgtcctgggggcttctccaacccctgctacggcaaaggcaactgcagcgatggggtccggggcaatggagcctgcctctgctttccagactacaagggtatcgcctgtcatatctgctccgacccaaagaagcatggagagcagtgccaggaggactgtggctgtgtccacggtctgtgtgacaaccgtcctggcagtggaggggtgtgccagcagggtacatgtgcccctggcttccaaggccgattctgcaatgaatccatgggaaactgtgggtccacagggctggcccagccctgccactcggacgctcactgtgttatccaggagggtgttgccaggtgcgtctgccatgatggttttgaaggtaatggcttctcctgcaaacgcagcaatccttgctcgcgcccagaccgtggtggctgctcagagaacgctgaatgtgtccctggagacctgggtacccaccactgcatttgccacaaaggctggagtggggatggccgcatctgtgtggccatcgatgaatgtgggctggacactcgaggtggctgccatgctgatgctctctgcagctatgtgggccctggacagagccgatgcacgtgtaagctgggcttcgctggaaatggctacgagtgcagccccattgacccctgtcgggtgggcaatggcggctgccacggcctggctacctgcaaagcagtggggggaggccagcgggtttgcacatgccctcctcattttggtggtgatggcttcagttgctatggagatattatccaggaactggaggcaaatgcccatttctctgccttctcccagtggttcaagaattcaagcatcactcttcctgccgacagcagagtcacagccctggtaccctctgagtccgccatccgtagactgagcctcgaggaccaggccttctggttgcagccaaagatgctgccagaactggccagggcccattttctccagggtgccttctcagaggaggagttggcccggttaaatggtcaacaagtagccacactgagtgccacgacacgctggcagatacacaacatcagtgggaaagtctgggtgcagaatgccactgtggatgtccccgacctccttgctaccaatggcatcctacacattgttagccaggtcttgttgcctccaagaggtgacatgcagaccgggcccgggttgctgcagcaactagatagtgtgcctgcctttcgcctcttcggagagcagctaaagcaccacaaactggtggcccaaatcgaggctgccaaagcctacaccatctttgtgcccacaaatcactctctggagacccagggcaacaacagtgtcctgggtatagacacggtgcgacatcacgtgatccttggggaggcactctctgtggaggtcctgcggaaagggggacaccgcaactccctcctgggccctgcccactggctggtcttctacaatcacagtggccagcctgaggtgaatcacatgccattggagggccctttgctggaagctcctggcagctccctcttcggactgtcggggatcttggcagtgggctccagccgttgcctgcacagccacgcagaggctcttcgggagaaatgcataaactgtacccggaaattccgctgtactcaaggcttccagctgcaggacacaccgaggaagagctgtgtctacagatctggattgtctttctcccggggctgttcctacacctgtgccaagaagatccaggtgcccgactgctgcccaggcttctttggtaccctgtgtgagccctgcccagggggtcttggtggagtgtgctcaggccatgggcagtgccaagacagattcctgggcaatggggaatgtcgctgccaagaggggttccatgggacagcctgtgagatgtgtgagctgggtcgctatggccccacctgctctggagtatgtgactgtgaccatgggctgtgccaggaggggctgcgcgggaatggaagctgtgtctgtcatgcgggctggcagggtctccgctgtgaccagaaaatcactgatcatcaatgtcccaagaagtgcgatcccaatgccaactgcatccaggactctgctggaatccctgcctgcgtctgtgctgcaggatactcgggcaacggcagctattgctcagaggtggatccctgtgcctctggccatgggggctgctcaccctatgccaactgtaccaaggtggctcctgggcagcggacatgcacctgccaggatggctacacgggcgatggagagctatgccaggaaataaacagctgtctcgtccacaatgggggctgtcatgttcatgctgagtgtatccccacaggcccccagcaggtctcctgcagctgccgtgagggctacagtggcgatggcattcagacttgcaagcttctggacccctgttcccagaacaatggaggctgcagtccttatgctgtgtgcaaaagcacgggagatggccagaggacgtgttcctgtgacgcaacacatactgtgggggacggcatcacctgtcatgggagagttggactggagctcctacggaacaagtatgcctccttcttcagcctccacctcctggaatacaaggagctcaagggtgacggacctttcactgtctttgtgccacatgctgacttaataagcaacatgtcacaggatgagctggctaggatacgggcccatcgccagcttgtgtttcgctaccacgtggttggctgccggaagctgtggagccaagagatgctggaccagggctatatcactacactgtctggacacactctgcgtgttagtgagagggagggcagcatttatctcaatgattttgctcgggtggtaagcagcgatctcgaggtggtgaacggtgtcctgcacttcattgaccacgtcctgctgccaccagatgtgctacactgggagtctggtgctatcccaatccctcagagaaatgtcactgccgctgctgaaagcttcggctacaagatcttcagccgcctattgacggtggctggcctcctgcccatgcttcaggatgcgtcccatcgccccttcacgatgctgtggcccacagattctgccctgcaagccttgcctcctgatagaaagaactggctcttccacgaggaccaccgtgacaaactggcagccattttgcggggtcatatgattcgcaacattgaggccttggcgtctgacctacccaacctgggccaactccgaaccatgcacggaaacaccatctctttctcctgcggtctcacccggcctggtgagctcatagtaggcgaagacgaggcccacattgtgcaacggcacttgacctttgaaggtggcctggcttatggcattgaccagctgctggagccacctgaccttggcgcccgctgtgatcgctttgaacctcagccactccaaatgaagacttgcagcatctgtgggctggagccaccctgtcctcggggctcacgggagcagggcagccctgagacctgctggcgtcactactccaagttctggacaactccattgcactccatctcaatgcgtggagcttactggataccgtctagtttttggaatcgtaaccacatgagtaggggctgccaccgaaactgtgtcaccaccgtctggaagcccagctgttgccctggtcactatggcattaactgccatgcttgccctggtggccccagaagcccctgcagtgaccatggcgtgtgcctggatggtatacgtggaagtgggcagtgtaattgccatccgggttttgctgggacagcctgtgaactctgtgccccaggtgcctttggaccccagtgccaagcttgccgctgtacccaacatggccgttgtgatgaaggtcttgggggctctggctcctgcttctgcgatgaaggctggactggggcacgctgtgaagtgcagctggagttgcagcctgtgtgcaccccaccgtgtgcaccccaggccgtgtgccgtctgggcaacagctgtgagtgcagcctgggctatgaaggggatggccgtgtgtgcacagtggcagacctgtgccagaaggggcatggtggctgcagtaagcatgccaactgcagccaggtggggacagtggtcacttgcacctgcctgcctgactatgagggtgacggttggagctgccgagctcgagacccctgcttggatggccaccgtggaggttgcagcgagcacgctgactgcctcaataccggtccgaacacacggcgctgtgaatgccacgtaggctacgtgggtgatgggctgcagtgtctagaggagcttgaaccccctgtggacagatgcttgggaggatcatcaccttgccacactgatgctttgtgcactgacctacatttccaggaaaaacaggctggtgtcttccacatccaggccaccagtggcccttatggtctgaccttctcagaggccaaggaagcgtgtgagggccagggagccgtccttgcttcactccctcaactctctgccgcccaacagctaggttttcatgtctgcttcgtgggctggttggccaacggctctgctgcccaccctgtcgtcaccccagcggcagactgtggtaataatcgtgtaggtgtagttagccttggggtccgtaagaacctctcggagctctgggatgcctactgttaccgcgtgcaagacgtggcttgccagtgtcgggccggcttcgtgggtgacgggatcagcacgtgcaacgggaaactgcttgatgtcttggccgccactgccaacttctccaccttctatgggatgctgctgggctatgccaatgccacccagagaggtcttgaatttatggatttcctggaggacgagctcacctacaagacactcttcgttcctgtcaacaaaggctttgtggacaacatgacgctgagtggcccagatctagaactccacgcttctaacgccacctttctgagtatcaatgccagccgggggacattgcttcctgcccactcaggtcttagcctcttcataagtgacacgggccctgacaacacttctctggttcctctggccccgggggcagttgtggttagccacgtcatcgtgtgggacatcatggctttcaacggcatcattcatgctctggccagccccctgctcatgcctccccagactagggcagtgctgggatctgagcctccacctgtggcattaagcttgggggttgtggtaacttctggaacactgctggggctggtggcgggagctctctacctgcgtgcccgaggcaaacctccaggtttcagcttctctgcctttcaggcagaagataatgcagacgatgacttctccccatggcaagaagggaccagcccaaccctggtttctgtccccaaccctgtttttggcagcagtgacatcttttgtgaaccctttgatgattcagtcctggaggaggacttccccgacacccagagggtcctcaaggtcaaatgatgagctggaatgaagcagaggctctgagaagacacagaccatttttattgcttgtcagggtggctgagggggccaatggtctgtccaggacaataaaagtgccctcagcggatgtgggccatgtcaccaa

SEQ ID NO: 68 - DNA, 8152 bp, Mus musculus
agttctcctggcactcatcagaggagttgaaatcgtgaggtcaccctgaggggagcctggcatggcaaggagcaagcttctcctcggcaagctgctgcctttaatcctcatcttcctggggttgcttgtgcaaaatgcctgctccccaactgaagccccagaactgacaaagagatgtgataagaagtctactctaaccatcaagacggagtgccagtcctgctcggttaacattgcagtcaagtgtccagacggctacatcaagattaccaatggcactgtcggggttcgagattgcaggtattcgcttaaaatccagtcctacgtcctggatatccctggatgccgccatatctgtaggaaggattacctgcaacctcaatgttgtccaggacactggggcccagactgcatggagtgtccaggaggagcaagggcaccgtgtggtggcaggggtgtttgtgatgaaggcatggaaggaaccggaagctgctcctgccgggcaggctttcgtggaacagcctgtgagaactgtgctgctgaagacgtttttggtcccaattgttcagcagtgtgcagttgtgtccatggtgtgtgcaacagcgggatcagtggtgacggaacttgtgagtgcttgtctgcgtacagggggcccaggtgtgacaagcccatccctgagtgtgcagccttgctctgcccagaaaactccagatgctcaccttccagcaaagacgaaaccaaactccagtgcaaatgccttcccagttacaaaggggacggccagacctgcaagcccatcaacccatgtttaaaaaacgtctgccaccctcacgcaagttgttcctacctgggaccaaatcggcacagttgtgtatgccaaaaaggctaccaaggagatggtcaagtgtgtctgccagtggacccctgccaaaccagctatggaaactgccctacaaagtctacggtgtgcagatatgatgggcctggacagtctcactgcgagtgtaaagaacattaccgaaattttgtacctggagtggggtgcagtatgacggacatctgtgaatcaaaaaacccatgtcataagaatgcgaactgcagcacagtctcaccaggccaaacccaatgcacttgccagaaaggttatgtgggtgatggcctgaactgctatggaaacatcatgcagagactcagagaattaaatactgaacccagaggaatgtggcaaggacagctgacttccttcatctcaatcctggacagaacttatgcctggccactgagcaacctgggacccttcaccgtgctgctgccctcagacaagggactaaaaggggttgatgtaaaggagctcttaatggacaaagaggctgctcggtactttgtgaagctccacatcattgctgggcaaatgagcacggagcagatgtataacttggacaccttctataccttgacgggaaagtcaggggaaatcatcaacaaggataaggacaatcaactaaagctcaaactttatggaagcaaaatagtgcaaattatccaggggaacatagttgcttcaaatgggcttgtgcatatcctcgacagagcgatggacaagatagagcccacacttgagagcaacccccagcaaaccattatgacaatgctacaaccaagatatggcaagttcagatctttgctagagaaaaccaatgttggacaagccctggagaagggtggtattgatgagccatacaccatttttgttccaagtaatgaagcattgagtaatatgacggctggtgtcctcgattacctcctgtctccagagggctctcggaaacttctggaactcgtcagataccacattgttgcatttacccagctggaagtggctactctggtttccacacttcacatccgaagcatggccaaccagatcatcacatttaacataagcagcaaagggcagattctggcaaacaacgtggccgtggatgaaaccgaggtcgctgccaaaaacggccgaatttacacactgaccggagttctcattcctccctccatcctcccgatccttccccaccgatgcaatgaaacaaagagagagatgaaactgggcacttgcgtgaggtgtttcatgaaaaactggagcaaatgtcctactaactcggagcccacggcaatcttcacaaacaagtgtttctatggcagcagagcatggaacctgaaaattggttgtgcccgatattgtgatgtgactgtggagataccaagatgctgtaaaggtttcttcggacctgactgcaacccatgtccaggaggcttcatgaatccgtgttctggaaatgggcagtgcatagacggcctcggtggcaatgggacctgcatctgtgaggatggcttccaaggttccaggtgtcagttctgctccaaacccaacagatatggacctcagtgtaacagaacctgccagtgtgttcatggaatatgtgataacagactggacagcgatggatcctgcctgccaggaacatgccgagagggcacagctgggaggttctgtgacaagcagacgtcagcctgtgggccctatatgcaattctgtcacatccatgccacctgtgaatacagcaatgagacagccagctgtgtctgcaatgacggctatgaaggagatggaactctctgttctaagaaggacccttgcctgggatcgacctccagaggaggatgtagtcctaacgcagaatgcatccaagctagcacggggacatacagttgtgtgtgccagcggggctggacagggaacggaagagactgtgtggagatcaacagctgcctgctgcctagctccggaggctgccatgacaacgccacctgtttatatgtaggccctgggcagaatgaatgtgaatgcaagaaaggatttcgaggaaacggaattgattgtgaacctatcatttcatgcttggaacaaatagagaaatgtcaccccctggccacctgtcaatatactttgtctggtgtttggagctgtgtttgccaagagggctatgaaggaaatggtgtcctgtgctatggaaatgtgctcatggaactgtcgtttctctctgaagcagctgtattttaccagtggatcaataatgcttctctgcagtccatgctgtctgccacctcgaatctcactgtcctcgtgccatccctgcaagccatcaaggacatggaccagaatgaaaaaagcttctggttgtcacggaacaacattccagccctgataaaataccacacactactaggcacatacagagtggctgatctgcagactctgccatcctctcacatgctagcgacatccttgcagggaagcttccttcgcctggacaaggcagatgggaatatcacaattgagggggcatcctttgtggatggtgacaatgcagccacaaacggggtggtccacatcatcaacaaggtgctaatcccccaaagaggtctaactggctccttacccagtctactcacccgtctggaacagatgcctgactattccatcttccggggctacatcattcattacaatctggcaagtgcaatcgaggctgcagatgcttatactgtatttgtgccaaacaatgaagccatcgaaagttatatcagggagaagaaagccacctctctaaaggaagacattcttcaataccacgtggtcctgggggaaaagctcctgaggaatgacttgcacaacggcatgcaccgagagacgatgctgggcttctcctacctcctcgccttctttctccacaatgaccagctgtatgtaaatgaagctccaataaactacaccaatgtggccactgacaagggagtgatccatggtctggagaaagttctggaaatcaagaagaataggtgtgacaataatgacaccattattgtgagagggaagtgtggaaagtgttcccagcaaacactctgcccgctcgagacaaaaccacttagtgagacgaggaaatgcatctattccgtttacttcatggggaagagatccattttcatcgggtgccagctacagtgtgtgagaaccatcattacaagtgcatgctgtgctggcttctttggcccacaatgccaagcctgccctgggaaaggtcagaatgtgtgctctgggaatggcttctgtctggacggcgtgaatggcactggcacatgcgagtgcgagcagggcttcaatgggacggcctgtgagacctgcacagaggggaagtatggtattcactgtgaccaagcatgctcttgtgtccatgggagatgtaaccaaggtccctcgggagatggctcctgtgactgcgatgtcggctggcgaggagtgaagtgtgacagcgagatcacaacagacaactgcaatgggacctgccacaccagtgccaactgccttctcgatccagatggcaaagcctcgtgcaaatgtgcggcaggattccaagggaacggaacggtctgcacagccatcaatgcctgtgagatcagcaatggagggtgctctgccaaggctgactgtaagagaaccatcccaggaagccgggtgtgtgtgtgcaaggcaggctataccggcgacggcatcgtgtgcctcgaaatcaacccgtgtttggagaaccacggcggctgtgacaggcatgcagagtgcacgcagacagggcccaaccaggccgtctgtaactgcttgcccaagtacactggagatggaaaggtctgcacactcatcaacgtctgcttaactaataacggcggctgcagtccatttgccttctgcaatcacacagaacaagatcaaaggacatgtacctgcaagccagactacacgggtgatggaatcgtctgccgtggcagcatccattcggaacttccaaagaatccctcaacgtcccagtatttcttccagttgcaggagcatgctgtccaagagcttgctggacctggccccttcaccgtgtttgtgccttcatctgactccttcaacagtgagtccaagcttaaagtctgggataaacagggcctcatgtcccagattctacggtatcacgtggtggcctgccagcagctactgctggagaacctaaaggtgatcacgagtgccacaaccctccaaggagagccaatttccatctctgtctctcaggacactgtgctcataaacaagaaggcgaaggttctgtccagtgacattatcagcaccaacggagtcatccacgtcatagacacattgttgtctccccaaaacttgcttatcacccccaaaggtgcctccggcagggttctgctaaaccttactacagtggcagcaaaccatggatataccaaattcagcaagttgatacaggactcaggcttgctgaaagtcatcactgaccccatgcataccccagtcactctcttctggcctacggacaaagccctccaagccttgcctcaggagcagcaggacttcctgttcaatgaagacaacaaagacaagctgaaggcatacctgaagttccacgtgatccgagataccatggctttagcttcagacctccccaggtctgcttcctggaagaccctgcagggttccgagctgagtgtgaggtgtggaactggcagtgatgtcggagagctctttctaaatggacaaatgtgcaggattatacagcggagactcttgtttgacggaggtgtggcctatggcattgactgcctgctaatggatcctaccgaaggtggccgatgtgacacttttactaccttcaatattccgggggaatgtgggagctgtttctccactcccagatgcccactacagagcaaaccaaagggtgtgaggaagaagtgtatctacaacccgttacctttcaggagggatgtggaaggctgtcagaacctgtgcaccctggtggtccatgtcccgcggtgctgcagtggttacttcatgccagactgtcaggcctgccctggaggaccagacacaccgtgtaacaaccggggcatgtgttatgatcagtacaaacccacaggacagtgccaatgtcatactggtttcaacgggacagcctgtgagctctgcttgcctgggagatttggtcctgactgtcaaccctgtggctgctctgagcatgggcagtgtgacgaggggatcacaggctccgggcagtgcctctgtgaagcagggtggacaggccgcttctgtgatgctcccacagttgtgattccagtgtgcatacctgcttgttccatgcacgcaacctgtatggagaacaacacatgtgtgtgtaacttgaattacgaaggtgacggaatcacatgcacagtcgtggatttctgcaaacagaacaatgggggctgtgcgaaggtcgccaagtgctcccagaagggcacacaagtctcctgcagctgccagaagggctacaagggggatggccacagctgcacagagatagacccctgtgcaaacggtgtcaatggtggatgtcatgagcatgctacctgcaggatgacgggcccaggcaagcagaagtgtgaatgtaaaagtcactatgtcggggatggacgggactgtgagcccgaacagctgccccttgaccgctgcttacaggacaacggacaatgccacccagatgccaactgtgtggaccttcatttccaggacactactgttggcgtattccacctacgctccccactgggccagtacaaactgacatttgacaaagccaaagaggcctgtgctaaggaagctgcatccatagccacctacaaccagctctcctatgcccagaaggccaagtatcacctctgctcagctggctggctggagagtgggcgggttgcctaccccacgatctacgcctctaagaaatgtgcaaacattgtggggattgtagactacggaaccaggaccaacaagagtgaaatgtgggacgtcttctgttacagaatgaaagatgttaactgcacctgcaaggcaggctacgtgggcgatggcttctcctgcaatgggaacctgctgcaagtcctcatgtccttcccctcactcacgaacttcctgacagaggtgctggttttttccaggagctcagcccaaggccgggcgtttttgaaacacctgactgacctgtccatcagtggcaccctgtttgtgccacagaacagtgggctaccgaaaaataagagcctgtctgggcgggacattgagcaccacctcactaatgtcaacgtctccttttacgatgaccttgtcaatggtaccgtcctgaagactaggctgggaagccaactgctcatcacctccagccaggaccagctccaccaagaggccaggttcgtggatggaagagccattctgcagtgggacatcattgcctctaacggggttctccatatcatttctgaacctttgaaagctcctcccacggccgcaacggctgcccactctggcctaggaacaggcatattctgcgctgtcgtcctggttactggtgcgattgctctggctgcctactcctacttccggctaaaccagagaacaactggcttccggcgttttgagtcagaagatgacattgatgccttggcctttggcaagcagcagcctgagagtatcacaaaccctttgtatgagacctcaacgccggcagcccccgagccctcctgtgaccccttcaccgactctggagaacgggagctggagaacagtgaccctcttggggcactgaggtcctgatgtgagaagccagccaacagccacagccacgtgagccctgagccatcacggttacacggtgactcccctgctccagctgtcacttggatcatttgttttaaagtatgataactcagaagccatacctcacccctctggttagtctggggttgtcgtcatgggtaaggggacatgtttccaggacaccagtatacctctgcctcctctgggccttcactgtggctctctgcaccttccagatggtgcttggcctgttctgccctatctgtgggtagctgtgaccctgtggtatctcctattaactgtaagcaccaaaggcaaggcttcatcccatatgttctgtattccagtacccagaagtacctgccacacgggtgcgctcaataaatgtggggcccgaaaaaaaaaaaa

(19) United States
(12) Patent Application Publication
(10) Pub. No.: US 2012/0277144 A1
(43) Pub. Date: Nov. 1, 2012

(54) NOVEL COMPOUNDS FOR MODULATING NEOVASCULARISATION AND METHODS OF TREATMENT USING THESE COMPOUNDS

(76) Inventor: Henricus Johannes Duckers, Delft (NL)

(21) Appl. No.: 13/505,544

(22) PCT Filed: Nov. 4, 2010
(86) PCT No.: PCT/NL10/50736

§ 371 Date: Jul. 10, 2012

(30) Foreign Application Priority Data
Nov. 4, 2009 (NL) PCT/NL2009/050663

(51) Int. Cl. A61K 38/17; A61P 9/10; C07H 21/02; C07K 14/47; A61P 7/02; C12N 15/63; A61P 35/00; A61P 27/02; A61P 9/00; A61K 31/713; C12N 15/12

(52) U.S. Cl. 514/1.9; 514/44 A; 514/13.3; 536/24.5; 530/350; 536/23.5; 435/320.1; 514/1.1; 514/19.3; 514/20.8

(57) ABSTRACT

The invention relates to a method for modulating neovascularisation of a tissue in a subject in need thereof, said method comprising administering to said subject a therapeutically effective amount of a compound or a combination of compounds selected from an isolated nucleic acid molecule comprising a gene selected from the group consisting of RIKEN c DNA S9430020K01, Agtrl1, Apelin, Stabilin 1, Stabilin 2, TNFaip8l1, TNFaip8 and FGD5, and homologues thereof; a gene product encoded by said genes, or encoded by homologues of these genes, and functional fragments thereof; an antibody or derivative thereof directed against a gene product of said genes, or encoded by homologues of these genes, and functional fragments thereof, said derivative preferably being selected from the group consisting of scFv fragments, Fab fragments, chimeric antibodies, bifunctional antibodies, intrabodies, and other antibody-derived molecules; an antisense molecule, in particular an antisense RNA or antisense oligodeoxynucleotide, an RNAi molecule (siRNA or mi RNA) or a ribozyme capable of binding under stringent hybridization conditions to a gene or an m RNA gene product of said genes and homologues thereof; a small molecule interfering with the biological activity of a gene product of said genes and homologues thereof, and a (glycol)protein, a hormone and other biologically active compounds capable of interacting with said genes and homologues thereof or with a gene product thereof.

### FIELD OF THE INVENTION

The present invention is in the field of products capable of modulating neovascularisation. More specifically, the invention relates to gene products capable of modulating neovascularisation and to compounds which interfere with said gene products or their production and thereby stimulate or inhibit neovascularisation. The invention also relates to the use of the disclosed compounds in the treatment of disorders, including, but not limited to, cardiovascular, cerebrovascular and peripheral artery diseases, and diseases characterized by pathological neovascularisation, including (but not limited to) cancer and diabetes.

### BACKGROUND TO THE INVENTION

In the developing embryo, the primary vascular network is established by in situ differentiation of mesodermal cells in a process called vasculogenesis. Vasculogenesis, the de novo formation of blood vessels from progenitor stem cells, can also occur in adults and involves the mobilization and differentiation of vascular progenitor cells, for example, from the bone marrow, to sites of active vessel growth. It is believed that all later processes involving the generation of new vessels in the embryo and neovascularisation in adults, are mainly governed by the sprouting or splitting of new capillaries from the pre-existing vasculature in a process called neoangiogenesis (Pepper et al., Enzyme & Protein, 1996 49:138-162; Breier et al., Dev. Dyn. 1995 204:228-239; Risau, Nature, 1997 386:671-674), but also by vasculogenesis mediated by circulating and local stem cells and progenitor cells. Neovascularisation is defined as the process of (neo)angiogenesis and vasculogenesis by which vessel formation and vascular healing is mediated. Neovascularisation is not only involved in embryonic development and normal tissue growth, repair, and regeneration, but is also involved in the female reproductive cycle, establishment and maintenance of pregnancy, and in repair of wounds and fractures. Arteriogenesis, the formation of large bore vessels containing smooth muscle cells, is thought to be a continuum of the neovascularisation process. In the adult, new vessel formation is thus a synergistic process of angiogenesis, vasculogenesis and arteriogenesis (after here collectively called neovascularisation).

In addition to angiogenesis and vasculogenesis (neovascularisation) as a normal physiological process, aberrant neovascularisation is involved in a number of pathological processes, notably tumor growth and metastasis, and other conditions in which blood vessel proliferation, especially of the microvascular system, is increased, such as diabetic retinopathy, psoriasis and arthropathies. Inhibition of neovascularisation is useful in preventing or alleviating these pathological processes. On the other hand, promotion of neovascularisation is desirable in situations where vascularisation is to be established or extended, for example (but not excluded to), following tissue or organ transplantation, or to stimulate establishment of perivascular and/or collateral circulation in tissue ischemia and/or infarction, such as in coronary heart disease, cerebrovascular ischemic disease, peripheral (stenotic) artery disease and thromboangitis obliterans All three processes of new blood vessel formation-angiogenesis, vasculogenesis, and arteriogenesis (collectively: ‘neovacularization’),—play a role in the response to ischemia and infarction.

The neovascularisation process is highly complex and involves the maintenance of the endothelial cells in the cell cycle, degradation of the extracellular matrix, migration and invasion of the surrounding tissue and finally, tube formation. The molecular mechanisms underlying the complex neovascularisation processes are far from being understood.

Because of the crucial role of neovascularisation in so many physiological and pathological processes, factors involved in the control of neovascularisation have been intensively investigated. A number of growth factors have been shown to be involved in the regulation of neovascularisation; these include fibroblast growth factors (FGFs), platelet-derived growth factor (PDGF), vascular endothelial growth factor (VEGF) and hepatocyte growth factor (HGF).

Factors that are capable of modulating neovascularisation are known in the art. A particular family of endothelial cell-specific growth factors, the vascular endothelial growth factors (VEGFs), and their corresponding receptors are believed to be primarily responsible for stimulation of endothelial cell growth and differentiation, and for certain functions of the differentiated cells. These factors are members of the PDGF/VEGF family, and appear to act primarily via endothelial receptor tyrosine kinases (RTKs, Flt/Flk, KDR).

There remains however a need in the art for additional factors that modulate vasculogenesis. It is an objective of the present invention to provide new compounds that modulate neovascularisation.

### SUMMARY OF THE INVENTION

The present invention is based on the identification of genes, which are involved in the regulation of vessel formation and arterial repair. Gene products of these genes can be used to induce or inhibit new vessel formation in tissues of a subject. Also compounds interfering with said genes or gene products can be used to stimulate vessel formation and arterial repair, or to impede aberrant or unwanted neovascularisation (diabetic retinopathy, inflammatory neovascularisation, atherosclerotic plaque stabilisation, or tumor angiogenesis) to halt disease progression. These genes and their products are also involved in the physiological arterial repair response following physical and inflammatory vascular damage and have been shown to modulate atherosclerosis progression and atherosclerotic plaque instability to plaque rupture and myocardial infarction.

The invention therefore provides in a first aspect a method for modulating neovascularisation of a tissue in a subject in need thereof, said method comprising administering to said subject a therapeutically effective amount of a compound or a combination of compounds selected from:
an isolated nucleic acid molecule comprising a gene selected from the group consisting of RIKEN cDNA 9430020K01, Agtrl1, Apelin, Stabilin 1, Stabilin 2, TNFaip8l1, TNFaip8 and FGD5, and homologues thereof;
a gene product encoded by a gene selected from the group consisting of RIKEN cDNA 9430020K01, Agtrl1, Apelin, Stabilin 1, Stabilin 2, TNFaip8l1, TNFaip8, and FGD5, or encoded by homologues of these genes, and functional fragments thereof;
an antibody or derivative thereof directed against a gene product of a gene selected from the group consisting of RIKEN cDNA 9430020K01, Agtrl1, Apelin, Stabilin 1, Stabilin 2, TNFaip8l1, TNFaip8, and FGD5, or encoded by homologues of these genes, and functional fragments thereof, said derivative preferably being selected from the group consisting of scFv fragments, Fab fragments, chimeric antibodies, bifunctional antibodies, intrabodies, and other antibody-derived molecules;
an antisense molecule, in particular an antisense RNA or antisense oligodeoxynucleotide, an RNAi molecule (siRNA or miRNA) or a ribozyme capable of binding under stringent hybridization conditions to a gene or an mRNA gene product of the genes selected from the group consisting of RIKEN cDNA 9430020K01, Agtrl1, Apelin, Stabilin 1, Stabilin 2, TNFaip8l1, TNFaip8, and FGD5 and homologues thereof;
a small molecule interfering with the biological activity of a gene product of a gene selected from the group consisting of RIKEN cDNA 9430020K01, Agtrl1, Apelin, Stabilin 1, Stabilin 2, TNFaip8l1, TNFaip8, and FGD5 and homologues thereof, and
a (glycol)protein, a hormone and other biologically active compounds capable of interacting with a gene or gene product selected from the group consisting consisting of RIKEN cDNA 9430020K01, Agtrl1, Apelin, Stabilin 1, Stabilin 2, TNFaip8l1, TNFaip8, and FGD5 and homologues thereof.

Preferably, the method of the present invention relates to a method for modulating neovascularisation of a tissue in a subject in need thereof, said method comprising administering to said subject a therapeutically effective amount of a compound or a combination of compounds selected from:
an isolated nucleic acid molecule comprising the gene RIKEN cDNA 9430020K01 and homologues thereof;
a gene product encoded by the gene RIKEN cDNA 9430020K01 or encoded by homologues of this gene, and functional fragments thereof;
an antibody or derivative thereof directed against a gene product of RIKEN cDNA 9430020K01 or encoded by homologue of this gene, and functional fragments thereof, said derivative preferably being selected from the group consisting of scFv fragments, Fab fragments, chimeric antibodies, bifunctional antibodies, intrabodies, and other antibody-derived molecules;
an antisense molecule, in particular an antisense RNA or antisense oligodeoxynucleotide, an RNAi molecule (siRNA or miRNA) or a ribozyme capable of binding under stringent hybridization conditions to a gene or an mRNA gene product of the gene RIKEN cDNA 9430020K01 and homologues thereof;
a small molecule interfering with the biological activity of a gene product of the gene RIKEN cDNA 9430020K01 and homologues thereof, and
a (glycol)protein, a hormone and other biologically active compounds capable of interacting with the gene RIKEN cDNA 9430020K01 and homologues thereof or with a gene product thereof,
   wherein said gene homologue has at least 60% sequence identity with the sequence of said gene.

In a preferred embodiment, the method for modulating neovascularisation of a tissue in a subject in need thereof is for:
treating or alleviating or preventing the risk of suffering from cardiovascular disease, improving arterial healing following physical damage (stenting, medical intervention), treating or alleviating or preventing atherosclerosis, treating or alleviating or preventing atherosclerotic plaque formation, treating or alleviating or preventing plaque destabilization (vulnerable plaque formation and rupture); treating or alleviating or preventing cancer, in particular tumor angiogenesis; treating or alleviating or preventing diabetic retinopathy or retina retinopathy or any condition associated with enhanced, aberrant, immature, accelerated and/or uncoordinated vessel growth resulting in leaky or hyperpermeable vessels;
treatment to induce arterial remodeling and arterial integrity/hyperpermeability;
treatment to stimulate re-endothelialisation of compounds, grafts and/or devices (valves, vascular grafts, endovascular prosthesis, intravascular stents) to reduce the risk of thrombus formation thereon.

In another aspect the invention provides a pharmaceutical composition comprising a therapeutically effective amount of at least one compound as defined above and a pharmaceutically acceptable excipient, carrier or diluent.

Preferably said compound in said pharmaceutical composition as defined above is:
an isolated nucleic acid molecule comprising a gene selected from the group consisting of RIKEN cDNA 9430020K01 and homologues thereof;
a gene product encoded by the gene RIKEN cDNA 9430020K01 or encoded by homologues of this gene, and functional fragments thereof;
an antibody or derivative thereof directed against a gene product of the gene RIKEN cDNA 9430020K01 or encoded by homologues of this gene, and functional fragments thereof, said derivative preferably being selected from the group consisting of scFv fragments, Fab fragments, chimeric antibodies, bifunctional antibodies, intrabodies, and other antibody-derived molecules;
an antisense molecule, in particular an antisense RNA or antisense oligodeoxynucleotide, an RNAi molecule (siRNA or miRNA) or a ribozyme capable of binding under stringent hybridization conditions to a gene or an mRNA gene product of the genes selected from the group consisting of RIKEN cDNA 9430020K01 and homologues thereof;
a small molecule interfering with the biological activity of a gene product of a gene selected from the group consisting of RIKEN cDNA 9430020K01 and homologues thereof, and
a (glycol)protein, a hormone and other biologically active compounds capable of interacting with a gene or gene product selected from the group consisting of RIKEN cDNA 9430020K01 and homologues thereof.

In another aspect, the invention provides a method of treating a subject, comprising administering to said subject a therapeutically effective amount of the pharmaceutical composition as defined above. The treatment is suitably indicated for:
the treatment or alleviation or prevention of the risk of suffering from cardiovascular disease, improving arterial healing following physical damage (stenting, medical intervention), treatment or alleviation or prevention of atherosclerosis, treatment or alleviation or prevention of atherosclerotic plaque formation, treatment or alleviation or prevention of plaque destabilization (vulnerable plaque formation and rupture); treatment or alleviation or prevention of cancer, in particular tumor angiogenesis; treatment or alleviation or prevention of diabetic retinopathy or retina retinopathy or any condition associated with enhanced, aberrant, immature, accelerated and/or uncoordinated vessel growth resulting in leaky or hyperpermeable vessels;
the treatment to induce arterial remodeling and arterial integrity/hyperpermeability;
the treatment to stimulate re-endothelialisation of compounds, grafts and/or devices (valves, vascular grafts, endovascular prosthesis, intravascular stents) to reduce the risk of thrombus formation thereon.

In another aspect, the invention provides a (therapeutic) compound selected from:
an isolated nucleic acid molecule comprising a gene selected from the group consisting of RIKEN cDNA 9430020K01, Agtrl1, Apelin, Stabilin 1, Stabilin 2, TNFaip8l1, TNFaip8 and FGD5, and homologues thereof;
a gene product encoded by a gene selected from the group consisting of RIKEN cDNA 9430020K01, Agtrl1, Apelin, Stabilin 1, Stabilin 2, TNFaip8l1, TNFaip8, and FGD5, or encoded by homologues of these genes, and functional fragments thereof;
an antibody or derivative thereof directed against a gene product of a gene selected from the group consisting of RIKEN cDNA 9430020K01, Agtrl1, Apelin, Stabilin 1, Stabilin 2, TNFaip8l1, TNFaip8, and FGD5, or encoded by homologues of these genes, and functional fragments thereof, said derivative preferably being selected from the group consisting of scFv fragments, Fab fragments, chimeric antibodies, bifunctional antibodies, intrabodies, and other antibody-derived molecules;
an antisense molecule, in particular an antisense RNA or antisense oligodeoxynucleotide, an RNAi molecule (siRNA or miRNA) or a ribozyme capable of binding under stringent hybridization conditions to a gene or an mRNA gene product of the genes selected from the group consisting of RIKEN cDNA 9430020K01, Agtrl1, Apelin, Stabilin 1, Stabilin 2, TNFaip8l1, TNFaip8, and FGD5 and homologues thereof;
a small molecule interfering with the biological activity of a gene product of a gene selected from the group consisting of RIKEN cDNA 9430020K01, Agtrl1, Apelin, Stabilin 1, Stabilin 2, TNFaip8l1, TNFaip8, and FGD5 and homologues thereof, and
a (glycol)protein, a hormone and other biologically active compounds capable of interacting with a gene or gene product selected from the group consisting of RIKEN cDNA 9430020K01, Agtrl1, Apelin, Stabilin 1, Stabilin 2, TNFaip8l1, TNFaip8, and FGD5 and homologues thereof, for use as a medicament in:
the treatment or alleviation or prevention of the risk of suffering from cardiovascular disease, improving arterial healing following physical damage (stenting, medical intervention), treatment or alleviation or prevention of atherosclerosis, treatment or alleviation or prevention of atherosclerotic plaque formation, treatment or alleviation or prevention of plaque destabilization (vulnerable plaque formation and rupture); treatment or alleviation or prevention of cancer, in particular tumor angiogenesis; treatment or alleviation or prevention of diabetic retinopathy or retina retinopathy or any condition associated with enhanced, aberrant, immature, accelerated and/or uncoordinated vessel growth resulting in leaky or hyperpermeable vessels;
the treatment to induce arterial remodeling and arterial integrity/hyperpermeability;
the treatment to stimulate re-endothelialisation of compounds, grafts and/or devices (valves, vascular grafts, endovascular prosthesis, intravascular stents) to reduce the risk of thrombus formation thereon.

In another aspect, the invention provides an isolated nucleic acid molecule comprising a sequence which has a sequence identity of at least 60% with in any one of FIGS. 11, 13, and 15-20. FIG. 11 (SEQ ID NO:1) provides the sequence of the murine RIKEN cDNA 9430020K01 gene. No function of this gene was hitherto known. The present inventors are the first to provide an industrial application for this gene. Also, industrial application of the other genes indicated herein is provided.

In another aspect, the invention provides a gene product of an isolated nucleic acid molecule as defined above or a vector comprising the nucleic acid molecule as defined above for use as a medicament.

Preferably, said a gene product or a vector as defined above is for use in the treatment of a patient suffering from a reduced revascularisation. Preferably, said use is for treating a patient who is suffering from cardiovascular ischemic disease, cerebrovascular ischemic disease and/or peripheral artery disease, as well as attenuate the progression of solid tumor formation, and metastasis formation, and promote the efficacy of cytostatic therapy.

In particularly preferred embodiments, the (therapeutic) compounds of the invention as defined above is for use in the treatment or alleviation or prevention of the risk of suffering from cardiovascular disease, improving arterial healing following physical damage (stenting, medical intervention), treatment or alleviation or prevention of atherosclerosis, treatment or alleviation or prevention of atherosclerotic plaque formation, treatment or alleviation or prevention of plaque destabilization (vulnerable plaque formation and rupture); treatment or alleviation or prevention of cancer, in particular tumor angiogenesis; treatment or alleviation or prevention of diabetic retinopathy or retina retinopathy or any condition associated with enhanced, aberrant, immature, accelerated and/or uncoordinated vessel growth resulting in leaky or hyperpermeable vessels; the treatment to induce arterial remodeling and arterial integrity and/or hyperpermeability; and/or the treatment to stimulate re-endothelialisation of compounds, grafts and/or devices (valves, vascular grafts, endovascular prosthesis, intravascular stents) to reduce the risk of thrombus formation thereon.

### BRIEF DESCRIPTION OF THE DRAWINGS

FIG. 1 shows the expression pattern of the RIKEN cDNA 9430020K01 gene in different adult mouse tissues.

FIG. 2 shows the expression level of the RIKEN cDNA 9430020K01 gene during embryonic mouse development at different days following fertilization α-axis).

FIG. 3 A-C show the results of a 2D matrigel analysis at 3 hours after subculturing of HUVEC cells as described in Example 1. (A) saline treated control (showing network); (B) sham/scrambled siRNA treated control (showing network); (C) targetted silencing of RIKEN cDNA 9430020K01 (showing monolayer). Knock down of RIKEN cDNA 9430020K01 results in a failure to develop a 2D matrigel neocapillary bed in cell culture. Cells did not show alignment of the cells and tube formation as could be seen in the control conditions (A, B).

FIG. 4 shows the RIKEN cDNA 9430020K01 mRNA expression in HUVECS, 96 hours after siRNA transfection in concentrations of 5-2.5-1 ng/ml, as described in Example 1.

FIG. 5 shows the effectiveness of siRNA knock down of RIKEN cDNA 9430020K01 RNA expression as described in Example 1. A: using specific targeting siRNAs compared to scrambled non targeting siRNA and a control measured at 24-48-72-96 hrs. The number of HUVEC cells over time (depicting cell growth) with scrambled siRNA (solid line); no siRNA (bold striped line) and siRNA targeting RIK943 (short striped line). RIK943 silencing results in a inhibition of endothelial cell growth in cell culture. Relative cell growth at 0 hrs the number of cells was set at 100%. (B-D) Injection of the 3 siRNAs as a mixture directed against RIKEN cDNA 9430020K01 in the eye of mice pups. The concentration of the siRNAs was 5 ng/ml. (A) down regulation of RIK943 mRNA expression in vivo as measured by qPCR of samples from the retina of mice following injection of siRNA. The Y-axis depicts the relative RNA expression level (at 0 hrs, expression is set at 100%) as measured by qPCR. Dark grey bars (right) represent siRNA RIK943, light gray bars (left) represent the sham siRNA (control). (C) SEM micrographs indicating that down regulation of RIK943 (right) results in a complete loss of retinal vessel formation in the eyes of newborn mice compared to control (left). (D). Quantification of the effect of silencing of RIK943 on the number of junctions (left graph), number of retinal vessels (middle graph) and total tube length of the formed retinal vessels (right graph) in newborn mice following knockdown of RIK943. Bar indications same as FIG. 5B.

FIG. 6 shows the results of a proliferation assay of Example 1. Proliferation of HUVEC cell cultures exposed to specific targeting siRNAs against RIKEN cDNA 9430020K01 gene is strongly inhibited compared to a sham inhibition (non targeting siRNAs) and a negative control. The Y-as depits the number of cells, the x-as depicts time following subculturing of 100,000 siRNA-treated cells.

FIGS. 7 A and B show the results of a Propidium iodide (PI) cell cycle assay. HUVECS undergo a cell cycle arrest in the G1 phase upon exposure to specific targeting siRNAs of Example 1. The Y-axis depicts the % of cells in the G1/Go phase (A), and S/M/G2 phase (B) as measured by flow cytometry.

FIG. 8 A-C show that the number of cells (HUVECS) exposed to specific siRNAs become apoptotic compared to a negative control or an inhibition with non targeting siRNAs (sham) as described in Example 1. The Y-axis depict the relative number of viable cells (A), pre-apoptotic (B) and apoptotic (C) cells in cell culture as measured by flow cytometry.

FIG. 9 shows the results of a migration assay after 72 hours upon on HUVECS exposed to specific RIKEN cDNA 9430020K01 targeting siRNAs compared to HUVECS which were exposed to a treatment with non-targeting siRNA and a negative control as described in Example 1. HUVECS which were exposed to specific RIKEN cDNA 9430020K01 targeting siRNAs displayed a significantly reduced migration activity.

FIG. 10 shows FGD5 function in human ECs, upper panel shows a sham adenovirus treated group, lower panel shows the FGD5 adenovirus transfected group. The columns show the effect of FGD5 overexpression on: tip and stalk cell formation in vitro in a coated-bead fibrin gel assay (ECs visualized by phalloidin staining) (A), new vessel formation in matrigel in vivo (B), and ECs proliferation on coated beads in vivo (hematoxylin/eosin stained cryosection) (C).

FIG. 11 shows SEQ ID NO:1 with underlined the sequences of the forward and reverse primers.

FIG. 12 shows the amino acid sequence of the protein encoded by SEQ ID NO:1 (RIKEN cDNA 9430020K01).

FIG. 13 shows the nucleotide sequence of murine FGD5 (genbank accession number NM_{—}172731.2).

FIG. 14 shows the alignment of murine protein encoded by the RIKEN cDNA 9430020K01 and its human homologue.

FIG. 15 shows the nucleotide sequence of murine Tnfaip8 (genbank accession number NM_{—}134131.1).

FIG. 16 shows the nucleotide sequence of murine Tnfaip8l1 (genbank accession number NM_{—}025566.3).

FIG. 17 shows the nucleotide sequence of murine Agtrl1 (genbank accession number NM_{—}011784.3).

FIG. 18 shows the nucleotide sequence of murine Apelin (genbank accession number NM_{—}013912).

FIG. 19 shows the nucleotide sequence of murine Stabilin 1 (genbank accession number NM_{—}138672.2).

FIG. 20 shows the nucleotide sequence of murine Stabilin 2 (genbank accession number NM_{—}138673.2).

FIG. 21 shows specific progenitor cell recruitment after Ischemia in a mouse model (see Example 2). (A) Following 3 days after a myocardial infarction, specifically cKit+/Flk+ endothelial progenitor cells are increased in the heart, whereas (B) Sca+/Flk+ cells numbers did not change in heart tissue after MI. 3 Days after hind limb ischemia, specifically Sca+/Flk+ cells increase in ischemic skeletal muscle (D), but cKit+/Flk+ levels do not change (C). Thus, cKit+/Flk1+ endothelial progenitor cells, which are involved in the (repair) response following an acute myocardium infarction, are specifically recruited from the circulation following myocardial infaction, but not following hind limb ischemia.

FIG. 22 shows Agtrl-1 expression on EPCs (endothelial progenitor cells) and apelin expression/levels after Ischemia in a mouse model (see Example 2). (A) Mean Agtrl-1 expression is relatively low in the different sub populations, except for the cKit+/Flk+ cells, which show a high expression. Circulating apelin levels, the natural ligand for Agtrl-1, are upregulated after myocardial infarction, but not after hind limb ischemia (B). mRNA and protein levels of apelin in the ischemic heart increase after myocardial infarction (C and E), but not after hind limb ischemia (D and F). Thus, Agtrl1 is specifically expressed in cKit+/Flk1+ endothelial progenitor cells (involved in the (repair) response following an acute myocardium infarction), and that following an acute myocardial infarction, apelin (the ligand of Agtrl1) is upregulated in the circulation, as well as in the myocardium at the protein and mRNA level.

FIG. 23 shows EPC mobilisation after apelin infusion in the mouse (see Example 2). Systemic infusion of apelin increases the number of cKit+/Flk+ endothelial progenitor cells in bone marrow (A) and blood (C). Apelin infusion did not change the number of Sca+/Flk+ cells in these tissues: B (bone marrow) and D (blood). Thus, systemic infusion of apelin specifically stimulates the number of cKit+/Flk1+ in the bone marrow as well as in the circulation.

FIG. 24 shows the left ventricular (LV) function after myocardial infarction followed by apelin treatment (see Example 2). Echocardiography images of non-treated (A), PBS (B) and apelin treated (C) mice 2 weeks after the induction of MI. Apelin treatment improved Fractional Shortening of the left ventricle compared to the other groups (D), without changes in left ventricular end diastolic diameter (E), indicating better contraction during systole. The conclusions of this experiment are that systemic treatment with Apelin improves cardiac function following an acute myocardial infarction.

FIG. 25 shows the scar formation after MI and apelin treatment (see Example 2). Masson trichrome stainings of nontreated (A), PBS (B) and apelin treated (C) mice 2 weeks after the induction of MI. Apelin treatment increases myocardial thickness of the border zone (D) and diminishes infarct length (E) compared to the other groups. Thus, apelin infusion reduces infarct size after myocardial infarction.

FIG. 26. Revascularisation after myocardial infarction and apelin treatment (see Example 2). Lectin stainings of nontreated (A), PBS (B) and apelin treated (C) mice 2 weeks after the induction of MI. The capillary density was increased in animals treated with apelin compared to PBS or untreated animals (D). Thus, apelin infusion stimulates new vessel formation following a myocardial infarction.

FIG. 27. FGD5 is specifically expressed in endothelial cells (see Example 3). (A) FGD5 expression during embryonic development of c57/bl6 mice from 8.5 dpc to 16.5 dpc, analyzed by qPCR of Flk1+ and Flk1− cells. FGD5 expression was upregulated in Flk1+ cells at all time points. (B) Whole mount in situ hybridization of zebrafish larvae at 24 hours revealed expression of FGD5 in the vasculature, including dorsal aorta, intersegmental vessels, and posterior cardinal vein. (C) qPCR analysis of FGD5 expression in various tissues of mature C57/bl6 mice (N=6 animals). Values represent mean±SEM. *P<0.05 aorta versus expression in various tissues. (D) qPCR analysis of the other FGD-family members (FRG, FGD1, FGD2, FGD3, FGD4, and FGD6) and the pattern of vascular specific CD31 or eNOS(N=4 animals). Values represent mean±SEM. *P<0.05 aorta versus expression in various tissues. (E) qPCR analysis of primary cell lines, including human endothelial cells (HUVECs, HAECs), compared to non-relevant cell types (Hela, sarcoma). Data obtained from 3 separate experiments. *P<0.05 HUVECs and HAECs versus hela and sarcoma cells. Values represent mean±SEM. (F) Immuno-histological staining of myocardium of mature C57/bl6 mice demonstrated co-localization of the FGD5 protein (green FITC signal) with the endothelial cell marker Isolectin IB4 (red Cy-3 signal). 400× magnification. (G) Immuno-histological staining of cryosections of aortas derived from mature C57/bl6 mice. FGD5 is detected in the endothelium and adventitia (green FITC signal). No FITC signal was detected after incubation with an isotypic control. 650× magnification. (*) Depicts luminal area.

FIG. 28. FGD5 inhibits angiogenesis in vitro and ex vivo (see Example 3). (A) Representative tube formation of HUVECs in a standard 2D Matrigel tube formation assay following FGD5 silencing or sham siRNA transfection. HUVECs were visualized by Calcein-AM uptake. 400× magnification. Quantitative analysis of the Matrigel assays show the effect of FGD5 knockdown (FGD5KD) on (B) total tube length, (C) number of tubes, and (D) number of junctions compared to control and sham-treated HUVECs (N=6 individual experiments). Values represent mean±SEM. *P<0.05 FGD5KD versus control and sham siRNA, #P<0.10 FGD5KD versus control and sham siRNA. (E) Representative tube formation of HUVECs in a standard matrigel assay following FGD5 overexpression using Ad-FGD5 or sham adenovirus (Sham Ad). HUVECs were visualized using Calcein-AM uptake. 400× magnification. Quantitative analysis of the Matrigel assays demonstrate the effect of FGD5 overexpression on (F) total tube length, (G) number of tubes, and (H) number of junctions compared to control and sham adenovirus transfected HUVECs (N=6 individual experiments). Values represent mean±SEM. *P<0.05 Ad-FGD5 versus control and sham Adenovirus. (I) Representative micrographs show microvascular sprouting of matrigel embedded aortic rings transfected with Ad-FGD5 or sham Adenovirus. 400× magnification. (J) Quantitative analysis of the aortic ring assays demonstrate the effect of FGD5 overexpression on the microvascular area (N=8 individual aortic explants). Values represent mean±SEM. *P<0.05 Ad-FGD5 versus sham adenovirus. (K) FGD5 expression impedes microvascular sprouting in a coated bead assay. Representative microvascular sprouting of HUVECs coated on cytodex beads in matrigel. HUVECs were transfected with Ad-FGD5 and compared to non-transfected and sham Adenovirus treated controls. 650× magnification (L) Representative micrographs of phalloidin-stained microvascular networks (Texas-red fluorescent signal) of the non-transfected, sham virus transfected, and Ad-FGD5 transfected groups. 650× magnification. (M) Quantitative analysis of the micrographs demonstrates the effect of FGD5 overexpression on the relative sprout area per bead, (N) and the number of beads with multiple sprouts. (O) Toluidine blue staining shows the effect of FGD5 on lumen formation in the formed vessels. 650× magnification. (P) Quantitative analysis of the micrographs shows the effect of Ad-FGD5 overexpression on the number of beads with lumen formation compared to non-transfected and sham Adenovirus transfected controls (N=6 separate experiments, analysis of 20 beads per group per experiment). Values represent mean±SEM. *P<0.05 versus control and sham Adenovirus. †P<0.05 versus control.

FIG. 29. FGD5 inhibits angiogenesis in vivo (see Example 3). (A) Representative macroscopic pictures of subcutaneously inoculated matrigel plugs containing HUVEC-coated cytodex beads in immunodeficient SCID mice at day 8. HUVECs were transfected with either Ad-FGD5, sham Adenovirus transfected or non-transfected cells (control). 20× magnification. (B) Representative macroscopic pictures of the matrigel plugs of the sham Adenovirus and the Ad-FGD5 group in which the lack of accumulation of erythrocytes within the plugs obtained from the Ad-FGD5 group is evident. 50× magnification. (C) Histological hematoxylin/eosine staining shows a decline in accumulation of HUVECs on beads coated with Ad-FGD5 transfected HUVECs versus sham Adenovirus transfected HUVECs. 400× magnification. (D) Quantitative analysis of micrographs show the effect of FGD5 on cell surface (mm2 per bead) in the Ad-FGD5 transfected versus sham Adenovirus transfected and non-transfected control. (E) Immuno-histological staining reveals accumulation of CD31/Cy3+ECs surrounding the cytodex beads, (F) which co-localized with the blue fluorescent DAPI signal. The contours of the cytodex bead were visualized by auto-fluorescence. 650× magnification. (G) Quantitative analysis of the cell-bead plug data show the effect of FGD5 on CD31+ surface area per bead mm2 in the Ad-FGD5 group versus sham Adenovirus transfected group and non-transfected control (N=8 animals per group, 20 beads per animal were analyzed). Values represent mean±SEM. *P<0.05 versus control and sham adenovirus group. (H) Representative micrographs of the developing retinal vasculature visualized by whole mount isolectin IB4 staining (green FITC signal). Ad-FGD5 injection severely impeded vascular development as compared to sham Adenovirus controls. 400× magnification. (I) High magnification (650×) micrographs show disruption of vessels and shortened sprouts. (J) Whole mount CD31 immuno-histological staining (red Cy-3 signal) shows similar patterns. Magnification 700×. (K) qPCR validation of FGD5 expression in Ad-FGD5 injected retinas, 3 days post injection, compared to sham virus injected control. Quantification of the vascular areas of our experiment shows that FGD5 overexpression induced a decrease in (L) number of tubes, (M) number of junctions, (N) total tube length (μm), (O) whereas no effect was observed in mean tube length (m) (N=32 pups). Values represent mean±SEM. *P<0.05 versus sham virus injected eye. (P) Flow cytometry evaluation of % Flk1+ cells in the retina transfected with ad-FGD5 or sham Adenovirus, 3 days post injection. Red histograph represents the isotypic control, blue histograph indicates Flk1+ signal. (O) Bar-graph shows % Flk1+ cells in the alive (7AAD−) cell population. (R) Dot blot graph of the % of dead cells (7AAD+), % apoptotic cells (Annexin V+), and % alive cells (7AAD−/Annexin V−). (S) Quantification of the percentage of apoptotic Annexin V+ cells in Flk1+ population (Data obtained from N=6 mice). *P<0.05 versus sham Adenovirus transfected. Evaluation of endogenous FGD5 function during retinal vascular development in postnatal wildtype c57/bl6 mice showed that specific endogenous expression of FGD5 in KDR+ endothelial cells correlates with high levels of cleaved caspase 3. Flowcytometric evaluation of the murine retina at 3, 8, and 14 days after birth showed; (T) a specific increase in the percentage of KDR+ cells that were FGD5+ as compared to the KDR− cells at day 14, and (U) a significant increase of cleaved caspase 3+ cells in the KDR+ population as compared to the KDR− population from day 8. (V) Dotblot graph showing FGD5 and cleaved caspase 3 signal in the KDR+ cell population in the murine retina at day 3, 8, and 14 after birth, with mock indicating the signal in the staining control. (W) In the KDR+ cell population, a significant higher number of FGD5+ cells were cleaved caspase 3+ as compared to FGD5− cells. (X) Mean intensity (MI) levels of FGD5 was significantly higher in KDR+ cells as compared to KDR− cells, and increased gradually during retinal vascular development. (Y) MIF levels of FGD5 was also higher in the cleaved caspase 3+ cell population as compared to the cleaved caspase 3− cells. *P<0.05, N=8 for each time-point assessed. SiRNA-mediated silencing of endogenous FGD5 during murine retinal development of the vasculature resulted in the persistence of excessive sprouts. (Z) Representative micrographs of the developing retinal vasculature visualized by whole mount isolectin IB4 staining (green FITC signal). SiFGD5 injection and resulted in the persistence of excessive sprouts and poor differentiation of veins and arteries by lack of pruning of the junctional vessels as compared to sisham injected controls. 500× magnification. Quantification of the vascular areas of our experiment shows that FGD5 silencing induced an increase in number of junctions and tubes, and total tube length (μm), whereas a decrease was observed in mean tube length (μm) (N=18 pups). Values represent mean±SEM. *P<0.05 versus sisham injected eye.

FIG. 30. FGD5 inhibits EC-proliferation and induces cell death (see Example 3). (A) Number of HUVECs transfected with Ad-FGD5 (black triangle) compared to sham Adenovirus transfected (white square), or non-transfected controls (black circle). (B) The graph shows the effect of FGD5 on 3-(4,5-dimethylthiazol-2-yl)-2,5-diphenyl tetrasodium bromide (MTT) processing in HUVECs. (C) Cell count of HUVECs treated with FGD5 targeting siRNA compared to control siRNA treated or non-treated controls, 3 days post transfection (Data was obtained from 4 separate experiments in triplicate). Values represent mean±SEM. *P<0.05 versus sham Adenovirus/sham siRNA and non-treated control. (D) Flow cytometry evaluation of apoptosis in HUVECs transfected with Ad-FGD5, sham virus, or non-transfected cells. (E) Quantification of total percentage of Annexin V+ cells (Data obtained from 4 different experiments). *P<0.05 versus sham Adenovirus transfected cells. Representative western blot analysis of p53 (F) and p21CIP1 (G) levels in Ad-FGD5 and sham Adenovirus transfected HUVECs. Graphs show Licor-quantified differences in band density (Data obtained from 3 separate experiments. Values represent mean±SEM). *P<0.05 versus sham Adenovirus transfected cells. (H) qPCR analysis of RNA expression of the Notch signalling pathway genes following FGD5 overexpression and silencing compared to the appropriate controls (Data was obtained from 6 separate experiments in duplicates). Values represent mean±SEM. *P<0.05 versus sham siRNA or sham Adenovirus treated cells.

FIG. 31. Hey1 mediates FGD5-induced p53-dependent apoptosis (see Example 3). (A) Number of HUVECs at 3 days following sham virus, Ad-FGD5, Ad-FGD5 with siRNA Hey1, Ad-FGD5 with sham siRNA, and Sham virus with sham siRNA treated groups. *P<0.05 versus sham virus and control. ‡P<0.05 versus Ad-FGD5 with siRNA Hey1. †P<0.05 versus Ad-FGD5 and Ad-FGD5 with siRNA Hey1. Representative immunoblots with corresponding graphs that show quantification of (B) p53 and (C) p21CIP1 band densities in the examined groups. *P<0.05 versus control, sham virus, and Ad-FGD5 with siRNA Hey1 knockdown (Data obtained from 4 different experiments in duplicates). Values represent mean±SEM. (D) Flow cytometry analysis of apoptosis in HUVECs transfected with sham virus, Ad-FGD5, and Ad-FGD5 with siRNA Hey1. Dead cells are PI+, apoptotic cells are Annexin V+, and alive cells are PI−/Annexin V−. (E) Quantification of the percentage of Annexin V+ cells. *P<0.05 versus Ad-FGD5 with siRNA Hey1, sham Adenovirus and control (Data obtained from 4 different experiments). Shown are values of the mean±SEM. (F) Analysis of cell cycle distribution of non-transfected HUVECs, and HUVECs transfected with sham Adenovirus, Ad-FGD5 or Ad-FGD5 with siRNA Hey1. Indicated regions represent from left to right the subG1, G1 and S+G2 fraction respectively. (G) Quantification of the percentages of cells in the subG1, G1, and S+G2 fractions in the different experimental groups (Data was obtained from 4 separate experiments in duplicates). Values represent mean±SEM. *P<0.05 versus Ad-FGD5 with siRNA Hey1 knockdown, sham virus, and control. (H) Representative micrographs show phalloidin-stained microvascular sprouting (Texas-red fluorescent signal) of HUVECs coated on cytodex beads in matrigel. HUVECs were transfected with Ad-FGD5, or Ad-FGD5 with siRNA Hey1 and compared to sham virus treated controls. 600× magnification. (I) Analysis of the relative sprout area per bead demonstrates the effect of Hey1 knockdown in FGD5 overexpressing cells. (N=3 separate experiments, analysis of 20 beads per group per experiment). Values represent mean±SEM. *P<0.05 versus sham virus. #P<0.05 versus Ad-FGD5. (J) Dot-blot graphs show apoptosis in HUVECs transfected with sham virus, Ad-FGD5, and Ad-FGD5 with siRNA p53. (K) Quantification of the percentage of Annexin V+ cells. *P<0.05 versus sham virus and FGD5 with siRNA p53 (Data obtained from 4 different experiments). Values represent mean±SEM. (L) Representative microvascular sprouting of HUVECs coated on cytodex beads in matrigel. HUVECs were transfected with Ad-FGD5 or Ad-FGD5 with siRNA p53 and compared to sham virus treated controls. 650× magnification. (M) Quantitative analysis of the effect of p53 knockdown in FGD5-overexpressing cells on the relative sprout area per bead (N=3 separate experiments, analysis of 20 beads per group per experiment). Values represent mean±SEM. *P<0.05 versus sham virus. #P<0.05 versus FGD5 with siRNA p53.

FIG. 32. FGD5 binds and activates cdc42 (see Example 3). (A) Western blot analysis of protein levels of cdc42, rac1, and RhoA, following FGD5 expression in HUVECs. (B) Co-immuno precipitation (IP) of FGD5 in cell lysates derived from FGD5 overexpressing HUVECs. Western blot analysis of the samples showed selective precipitation of cdc42, but not Rac1 or RhoA, whereas IP using mouse IgG isotype showed no effective precipitation. Shown are representative western blot samples from 3 different experiments. Chemo-luminescent measurement of the GTP-bound small G-proteins in cell lysates from Sham Ad or Ad-FGD5 transfected HUVECs, showing the levels of (C) GTP-Rho-A, (D) GTP-Rac1, and (E) GTP-cdc42 in response to serum activation (Data was obtained from 4 separate experiments in duplicates). Values represent mean±SEM. *P<0.05 versus sham virus. (F) Representative micrographs of 3 different experiments, showing peri-nuclear localization of FGD5 (red fluorescent signal; nucleus stained by blue DAPI) in the cytoplasm of HUVECs. FGD5 signal co-localizes with cdc42 (green fluorescent signal). 1000× magnification. (G) FGD5 (red signal) does not co-localize with the focal adhesion marker zyxin (green signal). 1000× magnification.

### DETAILED DESCRIPTION OF THE INVENTION

### Terminology

The term “neovascularisation” as used herein refers to both the combined process of vasculogenesis and angiogenesis. Vasculogenesis is the formation of blood vessels when there are no pre-existing blood vessels, in contrast to angiogenesis, which term refers to the development of blood vessels from existing ones. Vasculogenesis was first believed to occur only during embryologic development, although it is now known that the process also occurs in adult organisms. Vasculogenesis involves migration and differentiation of endothelial precursor cells (angioblasts) in response to local cues (such as growth factors and extracellular matrix) and the formation of new blood vessels (vascular trees). These vascular trees are then pruned and extended through angiogenesis. Circulating endothelial progenitor cells (derivatives of stem cells) are known to contribute, albeit to varying degrees, to neovascularisation.

The term “ischemic cardiovascular or cerebrovascular event” or short “ischemic event”, as used herein refers to an interruption of the blood supply to an organ or tissue. An ischemic event may often be the result of a blood cloth and in patients with atherosclerotic stenosis is most often caused when emboli dislodge from the atherosclerotic lesion. The resulting stenosis, or narrowing or blockage of an artery or other vessel due to this obstruction may result in a large number of adverse conditions, many of which have severe consequences for the subject. Ischemic cardiovascular or cerebrovascular events as referred to herein include, but are not limited to stroke/transient ischemic attack or cerebrovascular attack, myocardial infarction, myocardial ischemia (angina pectoris), any cardiomyopathy complicated by myocardial ischmia (for instance symptomatic aortic stenosis, HOCM), cerebral bleeding, peripheral (unstable) angina pectoris, claudicatio intermittens (peripheral atherosclerotic artery disease) and other major abnormalities occurring in the blood vessels. The term “abnormalities occurring in the blood vessels” includes reference to coronary and cerebrovascular events as well as to peripheral vascular disease. The term “ischemic cardiovascular or cerebrovascular event” is often the acute stage of a medical condition that is broadly encompassed by the term “cardiovascular, cerebrovascular and peripheral artery disease” (here collectively termed “cardiovascular disease”). Such diseases include cerebrovascular and also peripheral artery diseases.

The term “ischemia”, as used herein, refers to an absolute or relative shortage of the blood supply or an inadequate flow of blood to an organ, body part or tissue. Relative shortage refers to the discrepancy between blood supply (oxygen delivery) and blood request (oxygen consumption by tissue). The restriction in blood supply, generally due to factors in the blood vessels, is most often, but not exclusively, caused by constriction or blockage of the blood vessels by thromboembolism (blood clots) or atherosclerosis (lipid-laden plaques obstructing the lumen of arteries). Ischemia result in damage or dysfunction of tissue. Ischemia of the heart muscle results in angina pectoris, and is herein referred to as ischemic heart disease.

The term “cardiovascular disease” (CVD) generally refers to a number of diseases that affect the heart and circulatory system, including aneurysms; angina; arrhythmia; atherosclerosis; cardiomyopathies; cerebrovascular accident (stroke); cerebrovascular disease; congenital heart disease; congestive heart failure; coronary heart disease (CHD), also referred to as coronary artery disease (CAD), ischemic heart disease or atherosclerotic heart disease; dilated cardiomyopathy; diastolic dysfunction; endocarditis; heart failure; hypertension (high blood pressure); hypertrophic cardiomyopathy; myocardial infarction (heart attack); myocarditis; peripheral vascular disease; small vessel disease; and venous thromboembolism. As used herein, the term “cardiovascular disease” also encompasses reference to ischemia; arterial damage (damage to the endothelial lineage) due to physical damage (endartiectomie, balloon angioplasty) or as a result of chronic damage (including atherosclerosis); myocardial damage (myocardial necrosis); and myonecrosis. In general, any physiological or pathophysiological condition that elicits a neovascularisation response is encompassed by the term “cardiovascular disease” as used herein.

The term “pathological neovascularisation” refers to an unwanted state of neovascularisation. The term refers to unwanted neovascularisation in organs or tissues, for example (but not limited to) neovascularisation in tumors or in the pathological formation of new blood vessels in the retina and in other vascular beds in patients suffering from diabetes. The term also refers to atherosclerotic plaque destabilization (based on atherosclerotic plaques neovascularization).

The terms “polypeptide,” “peptide” and “protein” are used interchangeably herein to refer to a polymer of amino acid residues. The terms apply to amino acid polymers in which one or more amino acid residue is an analog or mimetic of a corresponding naturally occurring amino acid, as well as to naturally occurring amino acid polymers. Polypeptides can be modified, e.g., by the addition of carbohydrate residues to form glycoproteins. The terms “polypeptide”, “peptide” and “protein” include glycoproteins and proteins comprising any other modification, as well as non-glycoproteins and proteins that are otherwise unmodified.

The terms “affecting the expression” and “modulating the expression” of a protein or gene, as used herein, should be understood as regulating, controlling, blocking, inhibiting, stimulating, enhancing, activating, mimicking, bypassing, correcting, removing, and/or substituting said expression, in more general terms, intervening in said expression, for instance by affecting the expression of a gene encoding that protein and/or of the gene product itself.

The terms “subject” or “patient” are used interchangeably herein and include, but are not limited to, an organism; a mammal, including, e.g., a human, non-human primate, mouse, pig, cow, goat, cat, rabbit, rat, guinea pig, hamster, horse, monkey, sheep, or other non-human mammal; and a non-mammal, including, e.g., a non-mammalian vertebrate, such as a bird (e.g., a chicken or duck), an amphibian and a fish, and a non-mammalian invertebrate.

The term “homologous” as used herein refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared ×100. Homologous sequences (referred to herein as “homologues”), preferably have more than 50, 60, 70, 80, 90, 95, 96, 97, 98, 99, 99.5 or more percent sequence identity with one another. The genes and their sequences provided herein are the murine forms. The skilled person will appreciate that any mammalian and preferably human homologue is expressly intended to be included herein.

The term “sequence identity” refers to the degree of similarity between any given nucleic acid sequence or amino acid sequence and a target nucleic acid sequence or target amino acid sequence, respectively. The degree of similarity is represented as percent sequence identity. Percent sequence identity is calculated by determining the number of matched positions in aligned nucleic acid sequences, dividing the number of matched positions by the total number of aligned nucleotides, and multiplying by 100. A matched position refers to a position in which identical nucleotides occur at the same position in aligned nucleic acid sequences. Percent sequence identity also can be determined for any amino acid sequence. To determine percent sequence identity, a query nucleic acid or amino acid sequence is compared to a database nucleic acid or amino acid sequence using the BLAST 2 Sequences (Bl2seq) program from the stand-alone version of BLASTZ containing BLASTN version 2.0.14 and BLASTP version 2.0.14. This stand-alone version of BLASTZ can be obtained from the U.S. govermment's National Center for Biotechnology Information web site (World Wide Web at “ncbi” dot “nlm” dot “nih” dot “gov”). Instructions explaining how to use the Bl2seq program can be found in the readme file accompanying BLASTZ.

The term “pharmaceutically acceptable carrier” refers to a carrier for administration of a therapeutic agent, such as antibodies or a polypeptide, genes, and other therapeutic agents. The term refers to any pharmaceutical carrier that does not itself induce the production of antibodies harmful to the individual receiving the composition, and which may be administered without undue toxicity. Suitable carriers may be large, slowly metabolized macromolecules such as proteins, polysaccharides, polylactic acids, polyglycolic acids, polymeric amino acids, amino acid copolymers, and inactive virus particles. Such carriers are well known to those of ordinary skill in the art.

Pharmaceutically acceptable carriers in therapeutic compositions may contain liquids such as water, saline, glycerol and ethanol. Additionally, auxiliary substances, such as wetting or emulsifying agents, pH buffering substances, and the like, may be present in such vehicles. Typically, the therapeutic compositions are prepared as injectables, either as liquid solutions or suspensions; solid forms suitable for solution in, or suspension in, liquid vehicles prior to injection may also be prepared. Liposomes are included within the definition of a pharmaceutically acceptable carrier.

The term “therapeutically effective amount” as used herein refers to an amount of a therapeutic agent to treat, ameliorate, or prevent a desired disease or condition, or to exhibit a detectable therapeutic or preventative effect. The effect can be detected by, for example, chemical markers or antigen levels. Therapeutic effects also include reduction in physical symptoms, such as decreased body temperature. The precise effective amount for a subject will depend upon the subject's size and health, the nature and extent of the condition, and the therapeutics or combination of therapeutics selected for administration. Thus, it is not useful to specify an exact effective amount in advance. However, the effective amount for a given situation can be determined by routine experimentation and is within the judgment of the clinician. Specifically, the compositions of the present invention can be used to treat, ameliorate, or prevent the occurrence of a cardiovascular or cerebrovascular event in a subject and/or accompanying biological or physical manifestations.

For purposes of the present invention, an effective dose will be from about 0.01 mg/kg to 50 mg/kg or 0.05 mg/kg to about 10 mg/kg of the polynucleotide, polypeptide or antibody compositions in the individual to which it is administered.

The term “functional fragment” refers to a shortened version of the protein which is a functional variant or functional derivative. A “functional variant” or a “functional derivative” of a protein is a protein the amino acid sequence of which can be derived from the amino acid sequence of the original protein by the substitution, deletion and/or addition of one or more amino acid residues in a way that, in spite of the change in the amino acid sequence, the functional variant or derivative retains at least a part of at least one of the biological activities of the original protein that is detectable for a person skilled in the art. A functional variant is generally at least 50% homologous (preferably the amino acid sequence is at least 50% identical), advantageously at least 70% homologous and even more advantageously at least 90% homologous to the protein from which it can be derived. A functional variant may also be any functional part of a protein; the function in the present case being particularly but not exclusively the capacity to modulate neovascularisation. Preferably the amino acid sequence differs from the native protein sequence mainly or only by conservative substitutions. More preferably the protein comprises an amino acid sequence having 70%, 80%, 90% or more, still more preferably 95%, sequence identity with the native protein sequence and optimally 100% identity with those sequences. “Functional” as used herein means functional in mammals, preferably human patients.

The term “antibody” includes reference to antigen-binding peptides and refers to antibodies, monoclonal antibodies, to an entire immunoglobulin or antibody or any functional fragment of an immunoglobulin molecule. Examples of such peptides include complete antibody molecules, antibody fragments, such as Fab, F(ab′)2, complementarity determining regions (CDRs), VL (light chain variable region), VH (heavy chain variable region), and any combination of those or any other functional portion of an antibody peptide. The term “antibody” refers to a polypeptide substantially encoded by an immunoglobulin gene or immunoglobulin genes, or fragments thereof which specifically bind and recognize an analyte (antigen). However, while various antibody fragments can be defined in terms of the digestion of an intact antibody, one of skill will appreciate that such fragments may be synthesized de novo either chemically or by utilizing recombinant DNA methodology. Thus, the term antibody, as used herein, also includes antibody fragments such as single chain Fv, chimeric antibodies (i.e., comprising constant and variable regions from different species), humanized antibodies (i.e., comprising a complementarity determining region (CDR) from a non-human source) and heteroconjugate antibodies (e.g., bispecific antibodies).

The terms “inducing” or “stimulating” as used herein in the context of neovascularisation refer to improved growth of new blood vessels and includes reference to prevention of malformation of vessel structure.

### DESCRIPTION OF THE PREFERRED EMBODIMENTS

### Gene Products of the RIKEN cDNA 9430020K01 Gene

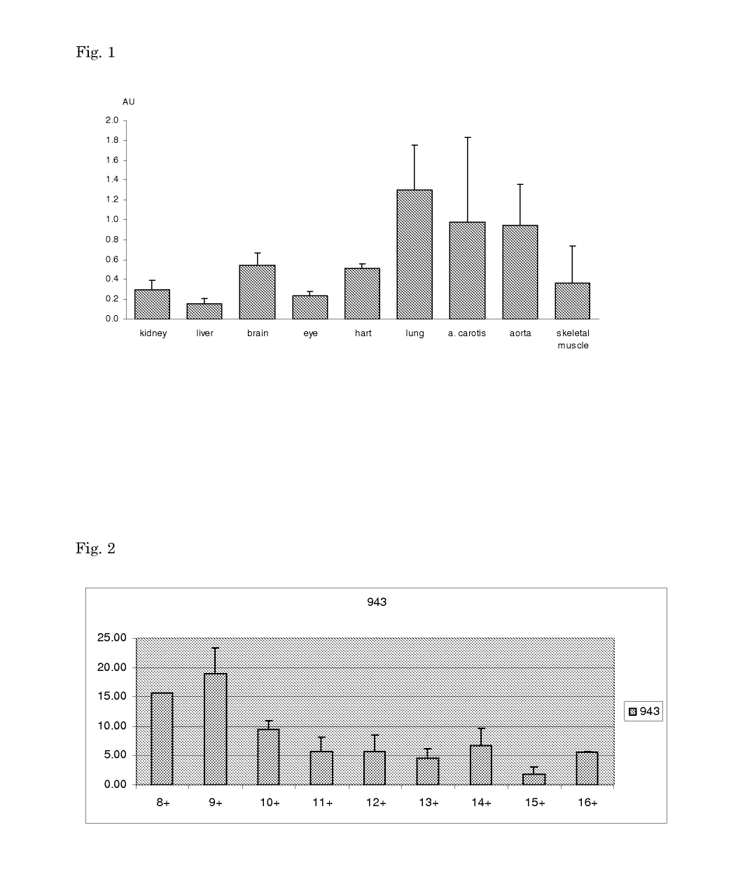

The RIKEN cDNA 9430020K01 gene was discovered upon a genome wide screen of various stages of vascular development during mouse embryogenesis which identified known as well as complete unknown and undocumented clones, designated EST clones or RIKEN clones. Some of these candidate genes did not have an amphibian orthologue and were further selected by their vasculature specific expression by comparing gene expression levels in isolated human aorta and highly vascularised tissue as opposed to other irrelevant organs by qPCR (see FIG. 1). Ensuingly, endothelial specific expression was further analysed using endothelial primary cell lines, more in particular HUVECs, and in vivo in the mouse. 9430020K01Rik was up-regulated during neovascularisation in the developing mouse (see FIG. 2) and was exclusively expressed in the vascular network.

The RIKEN cDNA 9430020K01 gene product was selected by the sheer potency to induce neovascularisation c.q. new vessel formation, equally, if not more potent, as compared to the most potent, hallmark neovascularisation regulatory gene (product) known to date, the VEGF family.

### Gene Products of the Agtrl1/Apelin Gene

The Agtrl1 gene encodes a member of the G protein-coupled receptor gene family. The encoded protein is an apelin receptor (also known as the APJ receptor) that inhibits adenylate cyclase activity and one of its known functions is that it plays a counter-regulatory role against the pressure action of angiotensin II by exerting hypertensive effect. Two transcript variants resulting from alternative splicing have been identified, both of which are indicated herein. Apelin is the protein ligand of the receptor.

Agtrl1 was identified in a transcriptome wide screen as a regulator of embryonic vasculogenesis. Whole mount in situ data of zebrafish larvae showed vasculature specific expression of Agtrl1 from 12 hours post-fertilization (hpf) onwards. Agtrl-1 knockdown was found to result in blood vessel malformations in zebrafish larvae. Morpholino-mediated silencing of Agtrl-1 also resulted in malformations of the heart and blood vessels in Fli::gfp mutant compared to uninjected larvae. The annotation “Agtrl1/apelin” as used herein refers to the combination of the receptor and its ligand and includes reference to ligand-mediated receptor activation. It was found that Agtrl1/apelin regulates mobilization of a specific EPC population. Agtrl1 was specifically expressed in endothelial cells and in the cKit+/Flk+ EPC population. Infusion of the Agtrl1 specific ligand apelin increased the cKit+/Flk+ EPC population in bone marrow and in blood.

The specific regulation of apelin protein and RNA levels after cardiac ischemia was studied and it was found that apelin is increased in ischemic tissue after myocardial ischemia (MI) on both protein level and RNA level, but not after 4 days hind limb ischemia (HLI).

It was further found that Agtrl1 regulates EPC recruitment in adult organisms in response to MI. An increase in cKit+/Flk+/Agtrl1+ EPC was found in ischemic tissue after MI, but not in HLI, whereas local injection of apelin recruits cKit+/Flk+ EPCs to Ischemic tissue in HLI.

### Gene Products of the Stabilin1 and 2 Genes

Stabilin1 and 2 are gene products which are expressed specifically in vivo in the endothelial cell lining of mice and zebrafish (Stabilin 2) and lymphogenic lining in the zebrafish. Morpholino-mediated gene silencing in zebrafish larvae resulted in malformations of the intersegmental vessels formation with aberrant side branch formation. Thus, the stabilins as referred to herein exhibit neovascularisation-inducing properties.

### Gene Products of the TNFaip8l1 Gene

Tumor necrosis factor alpha inducible protein 8 like 1 (TNFaip8l1) is expressed specifically in the endothelial cell lining of mice and zebrafish. The protein contains a death effector domain (DED), which suggests inhibition of caspase-mediated apoptosis. Morpholino-mediated gene silencing in zebrafish larvae resulted in malformations of the intersegmental vessels formation with aberrant side branch formation. In vitro knockdown of this gene in human umbilical vessel endothelial cells (HUVECs) showed similar effects. HUVECS that were silenced for TNFaip8l1 showed less proliferation and migration and proved to be less capable of forming a tubular network, compared to non silenced cells, all basic functions of endothelial during neovascularisation. These effects proved to be caused by the induction of apoptosis in the silenced cells. Hence, TNFaip8l1 exhibits anti-apoptotic functionality. It has now been found that the TNFaip8l1 gene is involved in vascular pruning, vessel maturation and remodeling, from an immature leaky neocapillary vascular bed in a functional hemodynamic relevant vascular bed, by removal of irrelevant non functional capillaries and maturation of existing vessels. TNFaip8l1 gene expression negatively regulates vessel pruning and vessel maturation (reactive vessel formation, inflammation, tumor neovascularisation, and ischemic neovascularisation). Silencing of the TNFaip8l1 gene or its gene products induces endothelial cell survival and facilitates neovessel formation in ischemic tissue and can thus be used as monotherapy or adjunctive therapy for ischemic disease. Therefore, it is advantageous to include a molecule capable of blocking a gene product of TNFaip8l1 in said pharmaceutical composition according to the invention.

### Gene Products of the TNFaip8 Gene

Tumor necrosis factor alpha inducible protein 8 (TNFaip8) is expressed specifically in the endothelial cell lining of mice and zebrafish. The protein contains a death effector domain (DED), which suggests inhibition of caspase-mediated apoptosis. Morpholino-mediated gene silencing in zebrafish larvae resulted in malformations of the intersegmental vessels formation with aberrant side branch formation. In vitro knockdown of this gene in human umbilical vessel endothelial cells (HUVECs) showed similar effects. HUVECS that were silenced for TNFaip8 showed less proliferation and migration and proved to be less capable of forming a tubular network, compared to non silenced cells, all basic functions of endothelial during neovascularisation. These effects proved to be caused by the induction of apoptosis in the silenced cells. Hence, TNFaip8 exhibits anti-apoptotic functionality. It has now been found that the TNFaip8 gene is involved in vascular pruning, vessel maturation and remodeling, from an immature leaky neocapillary vascular bed in a functional hemodynamic relevant vascular bed, by removal of irrelevant non functional capillaries and maturation of existing vessels. TNFaip8 gene expression negatively regulates vessel pruning and vessel maturation (reactive vessel formation, inflammation, tumor neovascularisation, and ischemic neovascularisation). Silencing of the TNFaip8 gene or its gene products induces endothelial cell survival and facilitates neovessel formation in ischemic tissue and can thus be used as monotherapy or adjunctive therapy for ischemic disease. Therefore, it is advantageous to include a molecule capable of blocking a gene product of TNFaip8 in said pharmaceutical composition according to the invention.

### Gene Products of the FGD5 Gene

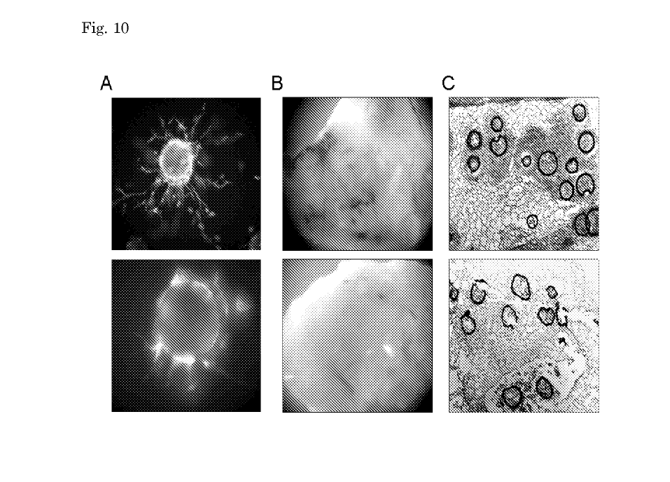

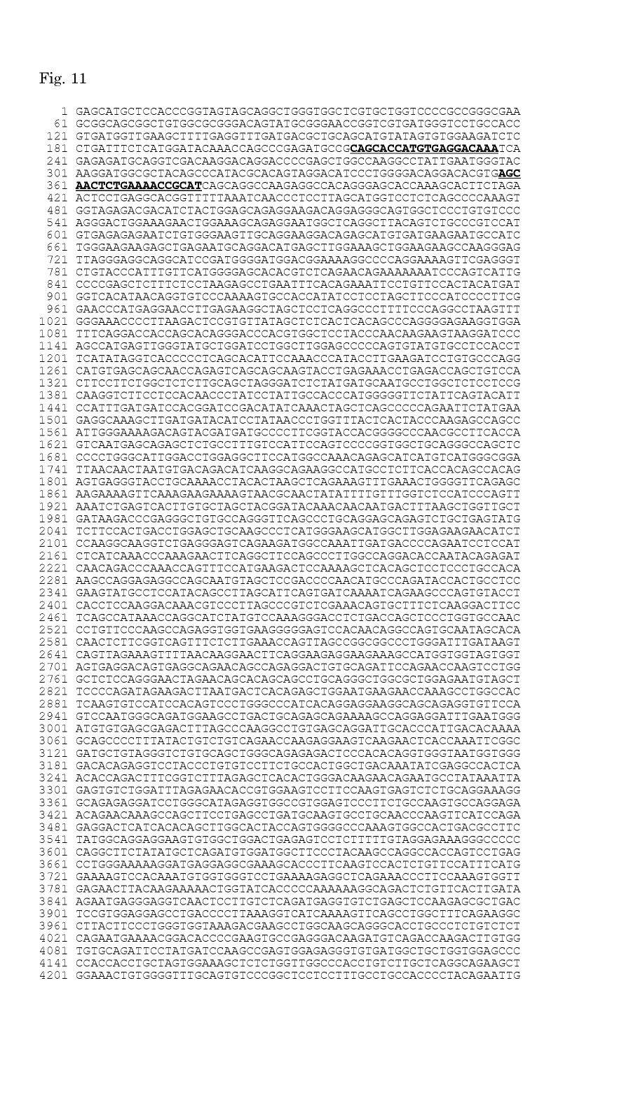

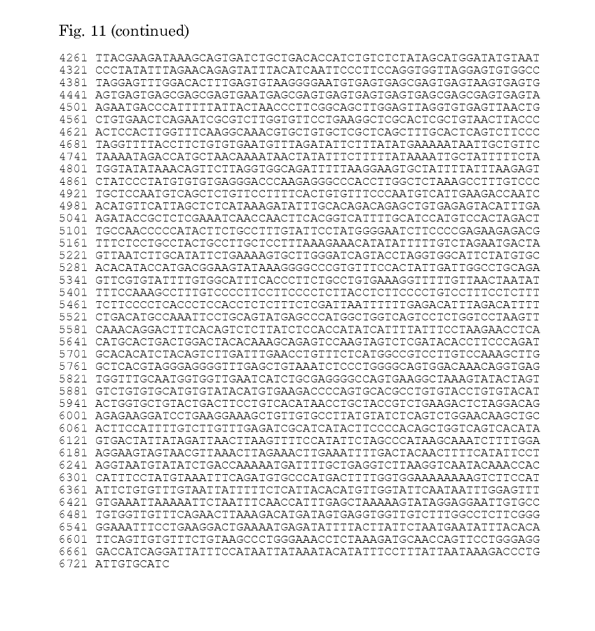

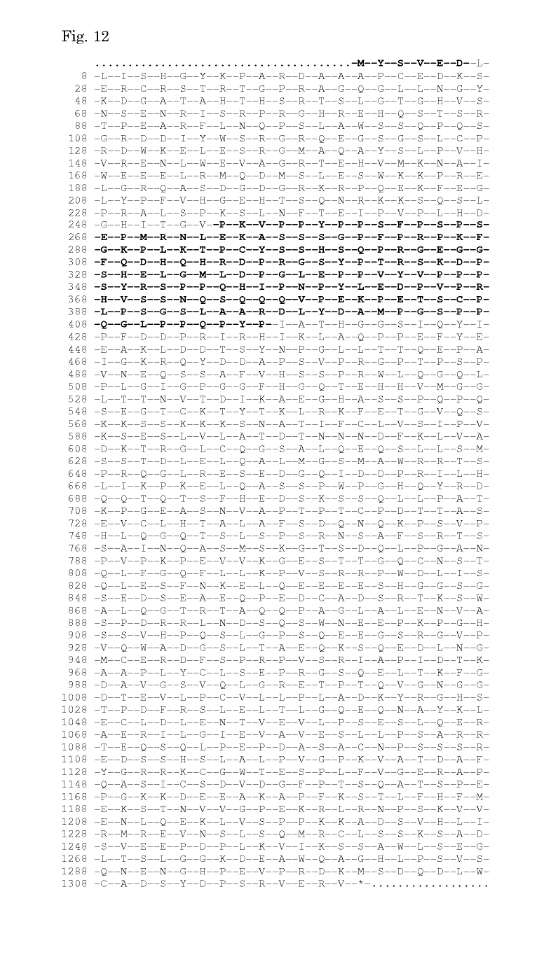

FGD5 is a gene that was selected after strenuous screening. Specific temporal and spatial expression of FGD5 in the vasculature was identified by whole mount in situ hybridization in developing zebrafish, and by qPCR and genome-wide microarray analysis in Flk1+ murine angioblasts, while qPCR analysis showed increased FGD5 expression in the aorta and carotid artery versus the other organs in adult C57bl/6 mice. In addition, high FGD5 expression levels were detected in primary human endothelial cells (ECs) compared to non-relevant cell lines. These data indicate that FGD5 is mainly expressed in the mature ECs and endothelial precusor cells. The FGD5 gene thus has an endothelial specific function. Knockdown of FGD5 expression by siRNA transfection significantly increased the capacity of human ECs to form new vessels in 2D matrigel in vitro, whereas adenoviral transfection of the FGD5 cDNA expressing vector inhibited new vessel formation. Similar effects were observed in an ex vivo aortic ring neovascularisation assay in which FGD5 cDNA overexpression in aortic segments isolated from C57bl/6 mice impeded sprouting of capillary structures. In addition, FGD5 adenovirus transfected ECs grown on sephadex beads showed severe inhibition in stalk and tip cell formation in an in vitro fibringel environment compared to sham transfected controls (FIG. 10A). In vivo, subcutaneous injection of a matrigel mix with sephadex beads coated with FGD5 transduced human ECs in SCID mice blocked new vessel formation (FIG. 10B) and ECs proliferation (FIG. 10C) in the matrigel plug compared to animals that received beads with shamvirus transfected ECs. This inhibitory function of FGD5 was explained by its effect on EC survival as FGD5 overexpression induced apoptosis, indicated by a rise in the percentage of annexin V+ cells in flow cytometric assessments, while siRNA knockdown of the gene decreased the number of annexin V+ (apoptotic and pre-apoptotic) cells. FGD5 inhibited EC proliferation and induced apoptosis in vitro by activating P53-P21 mediated cell cycle arrest, as indicated by Western blot analysis. Further studies showed that P53 induction was dependent on the regulation of the co-transcription factor HEY1 by FGD5, and siRNA blockage of the HEY1 pathway obliterated the FGD5 phenotype in ECs. HEY1 upregulation via FGD5 was associated with an increased expression of the receptors notch1/4 and their ligand DLL4, which point towards transcriptional activation of HEY1 via the notch1/4 signaling cascade.

FGD5 is a potential genetic regulator of neovessel formation, which is specifically expressed in mature and precursor endothelial cells. FGD5 is a potential regulator of early vascular pruning, as it induces cell cycle arrest and p53-p21-mediated apoptosis via the HEY1 cotranscription factor in the notch signaling cascade.

Without being bound by theory, we believe that this gene plays an important role in vessel remodeling, pruning, maturation and vessel stabilization. The FGD5 gene was shown to be a remarkably potent gene in EC selection, capillary pruning, and promotion of EC survival with facilitation of neocapillary formation upon silencing. In addition, overexpression of FGD5 in cell suspension in a matrigel plug potently prevents tumor vessel formation (as shown in FIG. 10 B/C). Thus, the FGD5 gene product as referred to herein inhibits neovascularisation.

The expression of gene FGD5 in mice and developing zebrafish model research was found to be restricted to endothelial precursor cells and mature ECs. In embodiments of the present invention, the aspects may be used and expressed in other cells and tissues as well. Gain and loss-of-function assays in vitro and in vivo demonstrated that FGD5 was involved in late vascular remodeling to remove redundant vascular structures. Our studies indicate that FGD5 functions as a Rho guanine-nucleotide exchange factor that binds and activates its direct target cdc42, and promotes Hey1-dependent p53-mediated apoptosis in endothelial cells. These findings identify FGD5 as a novel, critical regulator of vascular pruning during late vascular development by endothelial cell elimination. More details of embodiments of the therapeutic use of FGD5 and its gene products are provided in Example 3.

### Compounds of the Invention

The present invention provides in another aspect, compounds which are capable of modulating (by induction or inhibition) neovascularisation. This embodiment of the invention is inter alia based on the finding that in knockdown of RIKEN cDNA 9430020K01 in HUVECs cell culture remarkably inhibits cell proliferation by a cell-cycle arrest in the G1-phase. Also, concomitantly, more apoptosis was identified in the RIKEN cDNA 9430020K01-silenced endothelial cells. In line, tube formation by HUVECs in a 2D-matrigel assay was significantly attenuated by RIKEN cDNA 9430020K01 knockdown, while migration of HUVECs was decreased.

Gene function of RIKEN cDNA 9430020K01 was assessed in a murine retina model. Knockdown and overexpression of RIKEN cDNA 9430020K01 was induced by lentiviral infection of shRNA and cDNA expression vectors in the retina of C57bl/6 mouse pups directly after birth at day one. The RIKEN cDNA 9430020K01 gene is specifically expressed in the vasculature during embryonic vascularisation in murine development. Knockdown of RIKEN cDNA 9430020K01 impedes cell proliferation and increases apoptosis in EC in the retina in vivo.

Similar experiments were performed for the other genes and gene products described herein. For these other genes and gene products, similar or opposite effects were observed as indicated above, depending on the manner in which the gene or gene product modulates the neovascularisation process.

The invention therefore provides the following compounds or combination of compounds for use in modulating (by induction or inhibition) neovascularisation:
an isolated nucleic acid molecule comprising a gene selected from the group consisting of RIKEN cDNA 9430020K01, Agtrl1, Apelin, Stabilin 1, Stabilin 2, TNFaip8l1, TNFaip8 and FGD5, and homologues thereof;
a gene product encoded by a gene selected from the group consisting of RIKEN cDNA 9430020K01, Agtrl1, Apelin, Stabilin 1, Stabilin 2, TNFaip8l1, TNFaip8, and FGD5, or encoded by homologues of these genes, and functional fragments thereof;
an antibody or derivative thereof directed against a gene product of a gene selected from the group consisting of RIKEN cDNA 9430020K01, Agtrl1, Apelin, Stabilin 1, Stabilin 2, TNFaip8l1, TNFaip8, and FGD5, or encoded by homologues of these genes, and functional fragments thereof, said derivative preferably being selected from the group consisting of scFv fragments, Fab fragments, chimeric antibodies, bifunctional antibodies, intrabodies, and other antibody-derived molecules;
an antisense molecule, in particular an antisense RNA or antisense oligodeoxynucleotide, an RNAi molecule (siRNA or miRNA) or a ribozyme capable of binding under stringent hybridization conditions to a gene or an mRNA gene product of the genes selected from the group consisting of RIKEN cDNA 9430020K01, Agtrl1, Apelin, Stabilin 1, Stabilin 2, TNFaip8l1, TNFaip8, and FGD5 and homologues thereof;
a small molecule interfering with the biological activity of a gene product of a gene selected from the group consisting of RIKEN cDNA 9430020K01, Agtrl1, Apelin, Stabilin 1, Stabilin 2, TNFaip8l1, TNFaip8, and FGD5 and homologues thereof, and
a (glycol)protein, a hormone and other biologically active compounds capable of interacting with a gene selected from the group consisting of RIKEN cDNA 9430020K01, Agtrl1, Apelin, Stabilin 1, Stabilin 2, TNFaip8l1, TNFaip8, and FGD5 and homologues thereof or with a gene product thereof.

One of skill in the art will appreciate that the expression of RIKEN cDNA 9430020K01, Agtrl1, Apelin, Stabilin 1, and Stabilin 2 must be increased in order to induce or stimulate neovascularisation. Likewise, therapeutic administration of the gene products of these genes will stimulate neovascularisation. Compounds such as antibodies, antisense molecules and small molecules that interfere with the biological activity of the gene products of these genes are indicated for use in inhibiting neovascularisation.

One of skill in the art will appreciate that the expression of TNFaip8l1, TNFaip8, and FGD5 must be decreased in order to induce or stimulate neovascularisation. Likewise, therapeutic administration of the gene products of these genes will inhibit neovascularisation. Compounds such as antibodies, antisense molecules and small molecules that interfere with the biological activity of the gene products of these genes are indicated for use in inducing or stimulating neovascularisation.

The invention therefore provides as a therapeutic compound an antibody or derivative thereof (such as an scFv fragment, Fab fragment, chimeric antibody, bifunctional antibody, intrabody, and other antibody-derived molecule) directed against a polypeptide gene product of a neovascularisation modulating gene described herein. The antibodies of the present invention have the effect of interfering with the function of the protein such that, for instance, the ligand-receptor interaction or an enzyme function of the protein is blocked. Very suitable blocking antibodies are dendrimers. Such dendrimers may result in aggregation of the polypeptide gene products. Also generally envisioned herein are receptor antagonists.

The invention further provides as a therapeutic compound an antisense molecule, in particular an antisense RNA or antisense oligodeoxynucleotide, a morpholino, an RNAi molecule or a ribozyme binding under stringent conditions with a gene or a mRNA of a neovascularisation modulating gene described herein.

Preferably, the above compounds of the invention are used as a medicament. Medicaments of the invention can suitably be used for the treatment or prevention of pathological neovascularisation.

In another aspect the invention further provides a pharmaceutical composition for inhibiting neovascularisation comprising a compound according to the invention and a suitable excipient, carrier or diluent as explained above.

Preferably, the pharmaceutical composition for inhibiting neovascularisation according to the invention further comprises at least one gene product selected from TNFaip8l1, TNFaip8 and/or FGD5, and/or a compound selected from:
an antibody or derivative thereof directed against a gene product of a gene selected from RIKEN cDNA 9430020K01, Agtrl1, Apelin, Stabilin 1, and Stabilin 2, said derivative preferably being selected from the group consisting of scFv fragments, Fab fragments, chimeric antibodies, bifunctional antibodies, intrabodies, other antibody-derived molecules and antagonists of the gene products;
a small molecule interfering with the biological activity of gene product of a gene selected from RIKEN cDNA 9430020K01, Agtrl1, Apelin, Stabilin 1, and Stabilin 2;
an antisense molecule, in particular an antisense RNA or antisense oligodeoxynucleotide, an RNAi molecule or a ribozyme binding under stringent conditions with a gene or a mRNA of a gene selected from RIKEN cDNA 9430020K01, Agtrl1, Apelin, Stabilin 1, and Stabilin 2.

The present invention provides a pharmaceutical composition comprising:
a ligand that binds to a receptor encoded by any one of RIKEN cDNA 9430020K01, Agtrl1, Apelin, Stabilin 1, Stabilin 2, TNFaip8l1, TNFaip8, and FGD5;
a agonist of a gene product encoded by any one of RIKEN cDNA 9430020K01, Agtrl1, Apelin, Stabilin 1, Stabilin 2, TNFaip8l1, TNFaip8, and FGD5; and/or
a vector construct for overexpression of any one of RIKEN cDNA 9430020K01, Agtrl1, Apelin, Stabilin 1, Stabilin 2, TNFaip8l1, TNFaip8, and/or FGD5 in a mammalian cell comprising a coding nucleic acid sequence encoding any one of RIKEN cDNA 9430020K01, Agtrl1, Apelin, Stabilin 1, Stabilin 2, TNFaip8l1, TNFaip8, and FGD5 operably linked to a promoter that drives expression of said coding sequence in a mammalian cell, and a transcriptional termination sequence operably linked to the coding sequence;
   and a pharmaceutically acceptable carrier.

In an alternative second aspect, the present invention provides a pharmaceutical composition comprising:

a RIKEN cDNA 9430020K01, Agtrl1, Apelin, Stabilin 1, Stabilin 2, TNFaip8l1, TNFaip8, and/or FGD5-specific binding protein, a RIKEN cDNA 9430020K01, Agtrl1, Apelin, Stabilin 1, Stabilin 2, TNFaip8l1, TNFaip8, and/or FGD5 antagonist, a RIKEN cDNA 9430020K01, Agtrl1, Apelin, Stabilin 1, Stabilin 2, TNFaip8l1, TNFaip8, and/or FGD5 agonist scavenging compound, an antibody or small molecule inhibitor; and/or

a vector construct for silencing the gene encoding a RIKEN cDNA 9430020K01, Agtrl1, Apelin, Stabilin 1, Stabilin 2, TNFaip8l1, TNFaip8, and/or FGD5 gene product or its transcript by RNA interference comprising a coding nucleic acid sequence encoding an shRNA, siRNA or miRNA molecule capable of silencing the gene encoding RIKEN cDNA 9430020K01, Agtrl1, Apelin, Stabilin 1, Stabilin 2, TNFaip8l1, TNFaip8, and/or FGD5 or its transcript by RNA interference operably linked to a promoter that drives expression of said coding sequence in a mammalian cell, and a transcriptional termination sequence operably linked to the coding sequence;

and a pharmaceutically acceptable carrier.

Compounds as exemplified in the Examples below are suitable embodiments of compounds of the present invention.

### Medical Use

The present invention provides a method for inducing or stimulating neovascularisation comprising activating, increasing the activity and/or increasing the expression of a gene or product of a gene selected from the genes RIKEN cDNA 9430020K01, Agtrl1, Apelin, Stabilin 1 and Stabilin 2, and/or blocking, inhibiting the activity and/or inhibiting the expression of a gene or product of a gene selected from the genes TNFaip8l1, TNFaip8 and FGD5 in a subject in need thereof.

In preferred embodiments, the step of activating, increasing the activity and/or increasing the expression of a gene or product of a gene selected from the genes RIKEN cDNA 9430020K01, Agtrl1, Apelin, Stabilin 1 and Stabilin 2 is performed by:
contacting a gene or product of a gene selected from the genes RIKEN cDNA 9430020K01, Agtrl1, Apelin, Stabilin 1 and Stabilin 2 with a therapeutically effective amount of a ligand that binds to a gene or product of a gene selected from the genes RIKEN cDNA 9430020K01, Agtrl1, Apelin, Stabilin 1 and Stabilin 2 or an agonist of a gene or product of a gene selected from the genes RIKEN cDNA 9430020K01, Agtrl1, Apelin, Stabilin 1 and Stabilin 2, and/or
overexpressing in cells of said subject the gene selected from the genes RIKEN cDNA 9430020K01, Agtrl1, Apelin, Stabilin 1 and Stabilin 2.

In alternative preferred embodiments, the step of blocking, inhibiting the activity and/or inhibiting the expression of a gene or product of a gene selected from the genes TNFaip8l1, TNFaip8 and FGD5 is performed by:
contacting a gene or product of a gene selected from the genes TNFaip8l1, TNFaip8 and/or FGD5 with a therapeutically effective amount of an TNFaip8l1, TNFaip8 and/or FGD5-specific binding protein, a TNFaip8l1, TNFaip8 and/or FGD5 antagonist, a TNFaip8l1, TNFaip8 and/or FGD5 agonist scavenging compound, an antibody or small molecule inhibitor, and/or
silencing the gene encoding TNFaip8l1, TNFaip8 and/or FGD5 using RNA interference (RNAi).

The present invention also provides a method for inhibiting preventing neovascularisation comprising activating, increasing the activity and/or increasing the expression of a gene or product of a gene selected from the genes TNFaip8l1, TNFaip8 and FGD5, and/or blocking, inhibiting the activity and/or inhibiting the expression of a gene or product of a gene selected from the genes RIKEN cDNA 9430020K01, Agtrl1, Apelin, Stabilin 1 and Stabilin 2 in a subject in need thereof.

In preferred embodiments, the step of activating, increasing the activity and/or increasing the expression of a gene or product of a gene selected from the genes TNFaip8l1, TNFaip8 and FGD5 is performed by:
contacting a gene or product of a gene selected from the genes TNFaip8l1, TNFaip8 and FGD5 with a therapeutically effective amount of a ligand that binds to a gene or product of a gene selected from the genes TNFaip8l1, TNFaip8 and FGD5 or an agonist of a gene or product of a gene selected from the genes TNFaip8l1, TNFaip8 and FGD5, and/or
overexpressing in cells of said subject the gene selected from the genes TNFaip8l1, TNFaip8 and FGD5.

In alternative preferred embodiments, the step of blocking, inhibiting the activity and/or inhibiting the expression of a gene or product of a gene selected from the genes RIKEN cDNA 9430020K01, Agtrl1, Apelin, Stabilin 1 and Stabilin 2 is performed by:
contacting a gene or product of a gene selected from the genes RIKEN cDNA 9430020K01, Agtrl1, Apelin, Stabilin 1 and Stabilin 2 with a therapeutically effective amount of an RIKEN cDNA 9430020K01, Agtrl1, Apelin, Stabilin 1 and/or Stabilin 2-specific binding protein, a RIKEN cDNA 9430020K01, Agtrl1, Apelin, Stabilin 1 and/or Stabilin 2 antagonist, a RIKEN cDNA 9430020K01, Agtrl1, Apelin, Stabilin 1 and/or Stabilin 2 agonist scavenging compound, an antibody or small molecule inhibitor, and/or silencing the gene encoding RIKEN cDNA 9430020K01, Agtrl1, Apelin, Stabilin 1 and Stabilin 2 using RNA interference (RNAi).

The gene products and compounds as defined herein are indicated for therapeutic application. The present invention provides the therapeutic use of the compounds in ischemic heart disease, CNS and peripheral limb disease, as well as in oncological disease (e.g. tumours) and diabetes.

Several of the gene products and compounds have a pro-vasculogenic effect and therefore can stimulate local and circulating endothelial progenitor cells to create new arteries and stimulate repair of dysfunctional (atherosclerotic, immature or hyperpermeable) pre-existing arteries. This enables the treatment of cardiovascular ischemic disease or a final solution for patients currently not amenable for conventional therapy, for instance due to limited target vessels, high co-morbidity or small vessel disease (among others).

More specifically, the pro-vasculogenic gene products of the present invention can serve to stimulate the proper activation of the physiological response to a disease leading to an adequate vascular repair response. Preferred gene products include polynucleotides (DNA, RNA or synthetic nucleotides) and polypeptides. Preferred polynucleotides include RNA. The invention also encompasses a vector comprising a gene as identified herein or comprising a nucleic acid sequence encoding a functional fragment of the gene product as a medicament. Said vector can suitably be used in gene therapy, in which the genes identified herein are used for the design of dominant-negative forms which mimic the function of their wild-type counterparts following directed expression from a suitable vector in a target cell.

Said gene products also may be used for their anti vasculogenic effect and therefore can inhibit vessel formation. Inhibition of angiogenesis is useful in preventing or alleviating these pathological processes and/or remodel unorganized hyper-permeable arterial vascular beds (including in tumor angiogenesis and inflammatory neovascularisation) into a structured vascular tree with vascular integrity which is more amenable for pharmacotherapy and intervention.

The pro-vasculogenic gene products can therefore suitably be used for the treatment of a subject suffering from a disease which is characterized by a lower degree of neovascularisation. Such low degree of neovascularisation may occur in a specific organ or in a part of an organ affected by the disease. The term “low” in this context means lower than the degree of neovascularisation of the same organ or part of organ in a healthy person. In a preferred embodiment, said subject suffers from a cardiovascular disease. Therapy aimed to promote the formation of new vessels (neovascularisation) salvages compromised (ischemic) myocardial (or cerebrovascular) tissue, attenuates ongoing ischemic damage (with decrease of infarct size) and post myocardial adverse remodeling, leading eventually to long term preservation of myocardial function (and prevention of heart failure, reduction of cerebrovascular accidents, or alleviate claudicatio complaints and ischemic ulceration). Without being bound by theory, it is believed that endothelial progenitor cells (EPC) are recruited upon ischemia, for instance in myocardial infarction (AMI) and heart failure (CHF), although these cells are upregulated merely after 7-10 days after onset of the myocardial infarction. The vasculogenic gene products of the invention are not only involved in new vessel formation but also in the initiation of the vascular repair response of these EPC. Therefore expression of these vasculogenic gene products in the relevant cell population or locally at the site of ischemia (infusion in culprit coronary artery or intravenous infusion) is believed to improve prognosis and outcome of these AMI or CHF patients.

The compounds and compositions of the present invention can be used to improve vascular healing after physical damage, to improve maturation of premature vessels as well as reduce vascular permeability, and to modify atherosclerotic plaque stability, and to modulate vessel formation in solid tumors. Improving plaque stabilization can be used in aspects of preventing an acute myocardial infarction or cerebrovascular event, and may be obtained by enhancing or reducing vessel formation, depending on the therapeutic model. The modulation of vessel formation in tumors may comprise prevention or inhibition of vessel formation in order to retard cancer growth, but in other embodiments also the stimulation of vessel formation in cancers in order to make them more accessible to drugs.

In another aspect the invention provides a method of treating a subject, comprising administering to said subject the pharmaceutical composition of the invention in an amount effective to decrease (the risk of) a cardiovascular disease. Said pharmaceutical composition may be administered using topical, enteral, parenteral, transdermal, transmucosal, buccal (absorbed through cheek near gumline), inhalational or intracisternal routes or via vaginal suppositories etc.

The invention further provides a method of treating a subject, comprising administering to said subject the pharmaceutical composition for inhibiting neovascularisation according to the invention in an amount effective to decrease (the risk of) pathological neovascularisation.

The invention further provides a method of treating a subject, comprising administering to said subject the pharmaceutical composition for stimulating neovascularisation according to the invention in an amount effective to induce neovascularisation, preferably for inducing/modulate neovascularisation and vessel remodeling and maturation, for example (but not limited to) after tissue or organ transplantation, or to stimulate establishment of perivascular and/or collateral circulation in tissue ischemia or infarction, including cardiovascular ischemic, cerebrovascular disease and peripheral artery disease and thromboangitis obliterans, tumor angiogenesis, tumor metastasis, tumor therapy responsiveness, vessel permeabilisation and metastasis, and diabetic retinopathy.

In a preferred embodiment, said subject suffers from a cardiovascular disease. Therapy aimed to promote the formation, remodeling or maturation of new vessels (neovascularisation) salvages compromised (ischemic) myocardial or cerebrovascular tissue, attenuates ongoing ischemic damage (with decrease of infarct size or CVA) and post myocardial adverse remodeling, leading eventually to long term preservation of myocardial function (and prevention of heart failure). Without being bound by theory, it is believed that endothelial progenitor cells (EPC) are recruited upon ischemic myocardial infarction (AMI) and heart failure (CHF), although these cells are upregulated merely after 7-10 days after onset of the myocardial infarction. The vasculogenic gene products of the invention are not only involved in new vessel formation but also in the initiation of the vascular repair response of these EPC. Therefore expression of these vasculogenic gene products in the relevant cell population or locally at the site of ischemia (infusion in culprit coronary artery or intravenous infusion) is believed to improve prognosis and outcome of these AMI or CHF patients.

The invention thus also provides a method of treatment of a human comprising administering to said human the pharmaceutical composition for inducing neovascularisation according to the invention for alleviation or prevention of the risk of suffering a cardiovascular, cerebrovasuclar and peripheral artery disease, as well as oncological patients.

Methods as exemplified in the Examples below are suitable embodiments of methods of the present invention.

### Pharmaceutical Composition

The above compounds are suitably comprised in a pharmaceutical composition for stimulating neovascularisation further comprising at least one pharmaceutical acceptable additive like for example a pharmaceutically acceptable carrier, acceptable salts, an emulsifier, or a conservative.

Preferably, said pharmaceutical composition for stimulating neovascularisation further comprising at least one gene product selected from RIKEN cDNA 9430020K01, Agtrl1, Apelin, Stabilin 1, Stabilin 2, TNFaip8l1, TNFaip8, and/or FGD5. Angiotensin receptor 2 like 1 (Agtrl1) is one gene that showed vasculature restricted expression. In addition, subsequent loss-of-function studies in zebrafish by morpholino injections targeting Agtrl1 resulted in malformation of the cardiovascular system. Mammalian relevance in adult C57bl/6 mice for Agtrl1 was demonstrated by flow-cytometry: Agtrl1 is highly expressed in mature ECs and was also detected in cKit+/Flk1+ cells, a population of circulating EPCs. This population of cells was increased in the ischemic area after myocardial ischemia (P<0.001), but not after hind limb ischemia. Production of Apelin, the endogenous ligand of Agtrl1, was also augmented by 3-fold in the infarct region of the myocardium (P<0.01) and systemic infusion of apelin increased the recruitment cKit+/Flk1+EPCs to the blood circulation by two-fold (P<0.05). Injection of Apelin in ischemic myocardium resulted in increased homing of this specific population and a two-fold increase in neovascularisation of the ischemic tissue (P<0.01), consequently improving cardiac function and diminishing scarring. These findings indicate for the first time that increased levels of apelin in response to ischemic heart tissue could act as a specific chemo attractant for Agtrl1+EPCs, stimulating migration into the infarcted area and subsequently promoting neovascularisation and myocardial repair. Therefore, with a pharmaceutical composition comprising Agtrl1 and/or Apelin as therapeutically active compound is especially advantageous in treatments of cardiovascular diseases.

It is also advantageous to combine said gene product or said vector with an inhibitor which is a molecule capable of blocking the expression of a product of a gene selected from the group of RIKEN cDNA 9430020K01, Agtrl1, Apelin, Stabilin 1, Stabilin 2, TNFaip8l1, TNFaip8, and/or FGD5. These genes are described in WO 2009/075566, WO 2009/075579, WO 2009/075565 and WO 2009/075578. A skilled person can use techniques known in the art to make such inhibitors based on the sequence information provided in WO 2009/075566, WO 2009/075579, WO 2009/075565 and WO 2009/075578. In one embodiment of this aspect, the inhibitors are antibodies and/or antibody derivatives directed against the expression products of genes encoding the biomarkers. Therapeutic antibodies are for instance useful against gene expression products located on the cellular membrane and can be comprised in a pharmaceutical composition. Also, antibodies may be targeted to intracellular, e.g. cytoplasmic, gene products such as RNA's, polypeptides or enzymes, in order to modulate the activity of these products. Preferably, such antibodies are in the form of intrabodies, produced inside a target cell, preferably a plaque-forming cell including T-cells, endothelial cells, and smooth muscle cells, or cells that are found in atherosclerotic lesions, such as leukocytes, macrophages, foam cells, dendritic cells, and mast cells and T cells. In addition, antibodies may be used for deliverance of at least one toxic compound linked thereto to a target cell.

In a preferred embodiment of the present invention, the inhibitor is a small molecule capable of modulating the activity or interfering with the function of the protein expression product of the genes encoding RIKEN cDNA 9430020K01, Agtrl1, Apelin, Stabilin 1, Stabilin 2, TNFaip8l1, TNFaip8 and/or FGD5. In addition, small molecules can also be used for deliverance of at least one linked toxic compound to the target cell.

On a different level of inhibition, nucleic acids can be used to block the production of proteins by destroying the mRNA transcribed from RIKEN cDNA 9430020K01, Agtrl1, Apelin, Stabilin 1, Stabilin 2, TNFaip8l1, TNFaip8, and/or FGD5. This can be achieved by antisense drugs, ribozymes or by RNA interference (RNAi). By acting at this early stage in the disease process, these drugs prevent the production of a disease-causing protein. The present invention relates to antisense drugs, such as antisense RNA and antisense oligodeoxynucleotides, ribozymes and RNAi molecules, directed against RIKEN cDNA 9430020K01, Agtrl1, Apelin, Stabilin 1, Stabilin 2, TNFaip8l1, TNFaip8, and/or FGD5.

### Ribozymes

Trans-cleaving catalytic RNAs (ribozymes) are RNA molecules possessing endoribonuclease activity. Ribozymes are specifically designed for a particular target, and the target message must contain a specific nucleotide sequence. They are engineered to cleave any RNA species site-specifically in the background of cellular RNA. The cleavage event renders the mRNA unstable and prevents protein expression. Importantly, ribozymes can be used to inhibit expression of a gene of unknown function for the purpose of determining its function in an in vitro or in vivo context, by detecting the phenotypic effect.

One commonly used ribozyme motif is the hammerhead, for which the substrate sequence requirements are minimal. Design of the hammerhead ribozyme is well known in the art, as is the therapeutic uses of ribozymes. Ribozymes can for instance be prepared and used as described in U.S. Pat. No. 5,254,678. Ribozyme cleavage of HIV-I RNA is described in U.S. Pat. No. 5,144,019; methods of cleaving RNA using ribozymes is described in U.S. Pat. No. 5,116,742; and methods for increasing the specificity of ribozymes are described in U.S. Pat. No. 5,225,337. Preparation and use of ribozyme fragments in a hammerhead or hairpin structure is also known in the art. Ribozymes can also be made by rolling transcription.

The hybridizing region of the ribozyme may be modified or may be prepared as a branched structure. The basic structure of the ribozymes may also be chemically altered in ways familiar to those skilled in the art, and chemically synthesized ribozymes can be administered as synthetic oligonucleotide derivatives modified by monomeric units. In a therapeutic context, liposome mediated delivery of ribozymes improves cellular uptake.

Therapeutic and functional genomic applications of ribozymes requires knowledge of a portion of the coding sequence of the gene to be inhibited. Thus, for many genes, a nucleic acid sequence provides adequate sequence for constructing an effective ribozyme. A target cleavage site is selected in the target sequence, and a ribozyme is constructed based on the 5′ and 3′ nucleotide sequences that flank the cleavage site. Retroviral vectors are engineered to express monomeric and multimeric hammerhead ribozymes targeting the mRNA of the target coding sequence. These monomeric and multimeric ribozymes are tested in vitro for an ability to cleave the target mRNA. A cell line is stably transduced with the retroviral vectors expressing the ribozymes, and the transduction is confirmed by Northern blot analysis and reverse-transcription polymerase chain reaction (RT-PCR). The cells are screened for inactivation of the target mRNA by such indicators as reduction of expression of disease markers or reduction of the gene product of the target mRNA.

### Antisense

Antisense polynucleotides are designed to specifically bind to RNA, resulting in the formation of RNA-DNA or RNA-RNA hybrids, with an arrest of DNA replication, reverse transcription or messenger RNA translation. Antisense polynucleotides based on a selected sequence can interfere with expression of the corresponding gene.

Antisense polynucleotides are typically generated within the cell by expression from antisense constructs that contain the antisense strand as the transcribed strand. Antisense polynucleotides will bind and/or interfere with the translation of the corresponding mRNA. As such, antisense may be used therapeutically the inhibit the expression of oncogenes.

Antisense RNA or antisense oligodeoxynucleotides (antisense ODNs) can both be used and may also be prepared in vitro synthetically or by means of recombinant DNA techniques. Both methods are well within the reach of the person skilled in the art. ODNs are smaller than complete antisense RNAs and have therefore the advantage that they can more easily enter the target cell. In order to avoid their digestion by DNAse, ODNs and antisense RNAs may be chemically modified. For targeting to the desired target cells, the molecules may be linked to ligands of receptors found on the target cells or to antibodies directed against molecules on the surface of the target cells.

Antisense RNA includes reference to locked nucleic acid (LNA).

### RNAi

RNAi refers to the introduction of homologous double stranded RNA to specifically target the transcription product of a gene, resulting in a null or hypomorphic phenotype. RNA interference requires an initiation step and an effector step. In the first step, input double-stranded (ds) RNA is processed into nucleotide ‘guide sequences’. These may be single- or double-stranded. The guide RNAs are incorporated into a nuclease complex, called the RNA-induced silencing complex (RISC), which acts in the second effector step to destroy mRNAs that are recognized by the guide RNAs through base-pairing interactions. RNAi molecules are thus double stranded RNAs (dsRNAs) that are very potent in silencing the expression of the target gene. The invention provides dsRNAs complementary to the genes encoding the biomarkers of the present invention.

The ability of dsRNA to suppress the expression of a gene corresponding to its own sequence is also called post-transcriptional gene silencing or PTGS. The only RNA molecules normally found in the cytoplasm of a cell are molecules of single-stranded mRNA. If the cell finds molecules of double-stranded RNA, dsRNA, it uses an enzyme to cut them into fragments containing in general 21-base pairs (about 2 turns of a double helix). The two strands of each fragment then separate enough to expose the antisense strand so that it can bind to the complementary sense sequence on a molecule of mRNA. This triggers cutting the mRNA in that region thus destroying its ability to be translated into a polypeptide. Introducing dsRNA corresponding to a particular gene will knock out the cell's endogenous expression of that gene. This can be done in particular tissues at a chosen time. A possible disadvantage of simply introducing dsRNA fragments into a cell is that gene expression is only temporarily reduced. However, a more permanent solution is provided by introducing into the cells a DNA vector that can continuously synthesize a dsRNA corresponding to the gene to be suppressed.

RNAi molecules are prepared by methods well known to the person skilled in the art. In general an isolated nucleic acid sequence comprising a nucleotide sequence which is substantially homologous to the sequence of at least one of the genes encoding the biomarkers of the invention and which is capable of forming one or more transcripts able to form a partially of fully double stranded (ds) RNA with (part of) the transcription product of said genes will function as an RNAi molecule. The double stranded region may be in the order of between 10-250, preferably 10-100, more preferably 20-50 nucleotides in length.

RNAi molecules are preferably expressed from recombinant vectors in transduced host cells, hematopoietic stem cells being very suitable thereto.

Examples of RNAi molecules include siRNAs and miRNAs.

### Antibodies and Derivatives

The antibodies used in the present invention may be from any animal origin including birds and mammals (e.g., human, murine, donkey, sheep, rabbit, goat, guinea pig, camel, horse, or chicken). Preferably, the antibodies of the invention are human or humanized monoclonal antibodies. As used herein, “human” antibodies include antibodies having the amino acid sequence of a human immunoglobulin and include antibodies isolated from human immunoglobulin libraries (including, but not limited to, synthetic libraries of immunoglobulin sequences homologous to human immunoglobulin sequences) or from mice that express antibodies from human genes.

For some uses, including in vivo therapeutic or diagnostic use of antibodies in humans and in vitro detection assays, it may be preferred to use human or chimeric antibodies. Completely human antibodies are particularly desirable for therapeutic treatment of human subjects. Human antibodies can be made by a variety of methods known in the art including phage display methods described above using antibody libraries derived from human immunoglobulin sequences or synthetic sequences homologous to human immunoglobulin sequences. See also U.S. Pat. Nos. 4,444,887 and 4,716,111; and PCT publications WO 98/46645, WO 98/50433, WO 98/24893 and WO98/16654, each of which is incorporated herein by reference in its entirety.

The antibodies to be used with the methods of the invention include derivatives that are modified, i.e, by the covalent attachment of any type of molecule to the antibody such that covalent attachment. Additionally, the derivative may contain one or more non-classical amino acids.

In certain embodiments of the invention, the antibodies to be used with the invention have extended half-lives in a mammal, preferably a human, when compared to unmodified antibodies. Antibodies or antigen-binding fragments thereof having increased in vivo half-lives can be generated by techniques known to those of skill in the art (see, e.g., PCT Publication No. WO 97/34631).

In certain embodiments, antibodies to be used with the methods of the invention are single-chain antibodies. The design and construction of a single-chain antibody is well known in the art.

In certain embodiments, the antibodies to be used with the invention bind to an intracellular epitope, i.e., are intrabodies. An intrabody comprises at least a portion of an antibody that is capable of immuno-specific binding an antigen and preferably does not contain sequences coding for its secretion. Such antibodies will bind its antigen intracellular. In one embodiment, the intrabody comprises a single-chain Fv (“sFv”). In a further embodiment, the intrabody preferably does not encode an operable secretory sequence and thus remains within the cell.

Generation of intrabodies is well-known to the skilled artisan and is described for example in U.S. Pat. Nos. 6,004,940; 6,072,036; 5,965,371, which are incorporated by reference in their entireties herein.

In one embodiment, intrabodies are expressed in the cytoplasm. In other embodiments, the intrabodies are localized to various intracellular locations. In such embodiments, specific localization sequences can be attached to the intranucleotide polypepetide to direct the intrabody to a specific location.

The antibodies to be used with the methods of the invention or fragments thereof can be produced by any method known in the art for the synthesis of antibodies, in particular, by chemical synthesis or preferably, by recombinant expression techniques.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art. Examples of phage display methods that can be used to make the antibodies of the present invention include those disclosed in WO97/13844; and U.S. Pat. Nos. 5,580,717, 5,821,047, 5,571,698, 5,780,225, and 5,969,108; each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described below. Techniques to recombinantly produce Fab, Fab′ and F(ab′)2 fragments can also be employed using methods known in the art such as those disclosed in PCT publication No. WO 92/22324.

It is also possible to produce therapeutically useful IgG, IgA, IgM and IgE antibodies. For a detailed discussion of the technology for producing human antibodies and human monoclonal antibodies and protocols for producing such antibodies, see, e.g., PCT publication No. WO 98/24893. All references cited herein are are incorporated by reference herein in their entirety. In addition, companies such as Medarex, Inc. (Princeton, N.J.), Abgenix, Inc. (Freemont, Calif.) and Genpharm (San Jose, Calif.) can be engaged to provide human antibodies directed against a selected antigen using technology similar to that described above.

Recombinant expression used to produce the antibodies, derivatives or analogs thereof (e.g., a heavy or light chain of an antibody of the invention or a portion thereof or a single chain antibody of the invention), requires construction of an expression vector containing a polynucleotide that encodes the antibody and the expression of said vector in a suitable host cell or even in vivo. Once a polynucleotide encoding an antibody molecule or a heavy or light chain of an antibody, or portion thereof (preferably, but not necessarily, containing the heavy or light chain variable domain), of the invention has been obtained, the vector for the production of the antibody molecule may be produced by recombinant DNA technology using techniques well known in the art. Thus, methods for preparing a protein by expressing a polynucleotide containing an antibody encoding nucleotide sequence are described herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding an antibody molecule of the invention, a heavy or light chain of an antibody, a heavy or light chain variable domain of an antibody or a portion thereof, or a heavy or light chain CDR, operably linked to a promoter. Such vectors may include the nucleotide sequence encoding the constant region of the antibody molecule (see, e.g., PCT Publication WO 86/05807; PCT Publication WO 89/01036; and U.S. Pat. No. 5,122,464) and the variable domain of the antibody may be cloned into such a vector for expression of the entire heavy, the entire light chain, or both the entire heavy and light chains.

The expression vector is transferred to a host cell by conventional techniques and the transfected cells are then cultured by conventional techniques to produce an antibody of the invention. Thus, the invention includes host cells containing a polynucleotide encoding an antibody of the invention or fragments thereof, or a heavy or light chain thereof, or portion thereof, or a single chain antibody of the invention, operably linked to a heterologous promoter. In preferred embodiments for the expression of double-chained antibodies, vectors encoding both the heavy and light chains may be co-expressed in the host cell for expression of the entire immunoglobulin molecule, as detailed below.

A variety of host-expression vector systems may be utilized to express the antibody molecules as defined herein

In mammalian host cells, a number of viral-based expression systems may be utilized. In cases where an adenovirus is used as an expression vector, the antibody coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts. Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc.

Once an antibody molecule to be used with the methods of the invention has been produced by recombinant expression, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Further, the antibodies of the present invention or fragments thereof may be fused to heterologous polypeptide sequences described herein or otherwise known in the art to facilitate purification.

As stated above, according to a further aspect, the invention provides an antibody as defined above for use in therapy.

For therapeutic treatment, antibodies may be produced in vitro and applied to the subject in need thereof. The antibodies may be administered to a subject by any suitable route, preferably in the form of a pharmaceutical composition adapted to such a route and in a dosage which is effective for the intended treatment. Therapeutically effective dosages of the antibodies required for decreasing the rate of progress of the disease or for eliminating the disease condition can easily be determined by the skilled person.

Alternatively, antibodies may be produced by the subject itself by using in vivo antibody production methodologies as described above. Suitably, the vector used for such in vivo production is a viral vector, preferably a viral vector with a target cell selectivity for specific target cell referred to herein.

Therefore, according to a still further aspect, the invention provides the use of an antibody as defined above in the manufacture of a medicament for use in the treatment of a subject to achieve the said therapeutic effect. The treatment comprises the administration of the medicament in a dose sufficient to achieve the desired therapeutic effect. The treatment may comprise the repeated administration of the antibody.

According to a still further aspect, the invention provides a method of treatment of a human comprising the administration of an antibody as defined above in a dose sufficient to achieve the desired therapeutic effect. The therapeutic effect being the alleviation or prevention of the risk of suffering a cardiovascular or cerebrovascular event.

The diagnostic and therapeutic antibodies are preferably used in their respective application for the targeting of kinases or phosphatases, which are often coupled to receptor molecules on the cell's surface. As such, antibodies capable of binding to these receptor molecules can exert their activity-modulating effect on the kinases or phosphatases by binding to the respective receptors. Also transporter proteins may be targeted with advantage for the same reason that the antibodies will be able to exert their activity-modulating effect when present extracellularly. The above targets, together with signaling molecules, represent preferred targets for the antibody uses of the invention as more effective therapy and easier diagnosis is possibly thereby.

The diagnostic antibodies can suitably be used for the qualitative and quantitative detection of gene products, preferably proteins in assays for the determination of altered levels of proteins or structural changes therein. Protein levels may for instance be determined in cells, in cell extracts, in supernatants, body fluids by for instance flow-cytometric evaluation of immunostained target cells, preferably in blood or in endothelial progenitor cells (EPCs) or polymorphonuclear leukocytes (PMNs) present in said blood. Alternatively, quantitative protein assays such as ELISA or RIA, Western blotting, and imaging technology (e.g., using confocal laser scanning microscopy) may be used in concert with the antibodies as described herein for the diagnosis of an increased risk on cardiovascular or cerebrovascular events.

### Delivery Methods

Once formulated, the pharmaceutical compositions of the invention can be (1) administered directly to the subject; (2) delivered ex vivo, to cells derived from the subject; or (3) delivered in vitro for expression of recombinant proteins.

Direct delivery of the compositions will generally be accomplished by injection, either subcutaneously, intraperitoneally, intravenously or intramuscularly, or delivered to the interstitial space of a tissue. The compositions can also be administered into a plaque or lesion. Other modes of administration include topical, oral, catheterized and pulmonary administration, suppositories, and transdermal applications, needles, and particle guns or hyposprays. Dosage treatment may be a single dose schedule or a multiple dose schedule.

Methods for the ex vivo delivery and reimplantation of transformed cells into a subject are known in the art and described in e.g., International Publication No. WO 93/14778. Examples of cells useful in ex vivo applications include, for example, stem cells, particularly hematopoietic, lymph cells, macrophages, dendritic cells, or tumor cells.

Generally, delivery of nucleic acids for both ex vivo and in vitro applications can be accomplished by, for example, gene therapy using a vector including a virus, dextran-mediated transfection, calcium phosphate precipitation, Polybrene® mediated transfection, protoplast fusion, electroporation, encapsulation of the polynucleotide(s) in liposomes, and direct microinjection of the DNA into nuclei, all well known in the art.

Various methods are used to administer the therapeutic composition directly to a specific site in the body. For example, a target location is located and the therapeutic composition injected in the target directly. Alternatively, arteries which serve target location are identified, and the therapeutic composition injected into such an artery, in order to deliver the composition directly into the target location. The antisense composition is directly administered to the surface of an atherosclerotic lesion, for example, by topical application of the composition. X-ray imaging is used to assist in certain of the above delivery methods.

Receptor-mediated targeted delivery of therapeutic compositions containing an antisense polynucleotide, subgenomic polynucleotides, or antibodies to specific tissues is also used. Receptor-mediated DNA delivery techniques are well known in the art. Preferably, receptor-mediated targeted delivery of therapeutic compositions containing antibodies of the invention is used to deliver the antibodies to specific tissue.

Pharmaceutical compositions containing antisense, ribozyme or RNAi polynucleotides are administered in a range of about 100 ng to about 200 mg of polynucleotides for local administration in a gene therapy protocol. Concentration ranges of about 500 ng to about 50 mg, about 1 g to about 2 mg, about 5 g to about 500 g, and about 20 g to about 100 g of polynucleotides can also be used during a gene therapy protocol. Factors such as method of action and efficacy of transformation and expression are considerations which will affect the dosage required for ultimate efficacy of the polynucleotides. Where greater expression is desired over a larger area of tissue, larger amounts of polynucleotides or the same amounts readministered in a successive protocol of administrations, or several administrations to different adjacent or close tissue portions of, for example, an atherosclerotic site, may be required to effect a positive therapeutic outcome. In all cases, routine experimentation in clinical trials will determine specific ranges for optimal therapeutic effect. A more complete description of gene therapy vectors, especially retroviral vectors, is contained in U.S. Ser. No. 08/869,309, which is expressly incorporated herein.

### Diagnostic Methods

The genes and gene products identified herein are associated with neovascularisation processes and are therefore suitable for diagnostic purpose. The present invention therefore also provides as embodiments thereof a method of diagnosis or prognosis of cardiovascular disease in a subject, preferably cardiovascular disease associated with arterial damage or myocardial damage, even more preferably associated with ischemia. Such a method comprises the step of detecting in a sample of a circulation fluid of said subject an increase or decrease in the gene expression level of at least one gene and even more preferably at least 2, 3, 4, 5, 6, 7, or all genes selected from the group consisting of RIKEN cDNA 9430020K01, Agtrl1, Apelin, Stabilin 1, Stabilin 2, TNFaip8l1, TNFaip8, and FGD5 and human homologues thereof, preferably in endothelial progenitor cells (EPCs) in said sample, relative to a healthy control. Very suitable, the increase or decrease in the gene expression level is detected by detection of an mRNA or protein, i.e., the expression product of said one or more genes. The genes and gene products identified herein can thus be used as biomarkers for diagnosis or prognosis of cardiovascular disease in a patient, said biomarker comprising the expression product of a gene selected from the group consisting of RIKEN cDNA 9430020K01, Agtrl1, Apelin, Stabilin 1, Stabilin 2, TNFaip8l1, TNFaip8, and FGD5 the expression of which is regulated during vasculogenesis. The expression of the biomarker may be an upregulated or downregulated expression product, relative to values observed in a healthy control subject, and may for instance be upregulated or downregulated during vasculogenesis compared to angiogenesis. The biomarker may thus be a protein or RNA molecule expression product of the genes identified herein, or may be a protein profile or RNA profile. The use of said biomarkers for the diagnosis or prognosis of ischemia in a subject is expressly considered. Alternatively, the biomarkers may be used as surrogate end-point markers for determining the efficacy of therapeutic methods described herein. The expression of one gene may be measured but preferably a combination of the expression of at least 2, 3, 4, 5, 6, 7, or all genes selected from the group consisting of RIKEN cDNA 9430020K01, Agtrl1, Apelin, Stabilin 1, Stabilin 2, TNFaip8l1, TNFaip8, and FGD5 is determined such as to provide a profile of gene expressions for the referred genes.

The invention further contemplates and provides a method for the diagnosis or prognosis of ischemia in a subject, comprising detecting in the blood of said subject a biomarker according as described above. Such a method may suitably be performed by using a microarray (in particular comprising specific binding partners that bind specifically to at least two biomarkers as defined above bound to a solid support), by using tandem mass spectrometry (MS-MS), by MALDI-FT mass spectrometry, MALDI-FT-ICR mass spectrometry, MALDI Triple-quad mass spectrometry or immunoassay.

Kits of parts for performing a diagnostic method according to the invention are also envisioned herein. Such kits comprise at least one biomarker as defined above or a specific binding partner that binds specifically to said biomarker, said kit of parts optionally further comprising one or more of the following:
at least one reference or control sample;
information on the reference value for the biomarker;
at least one test compound capable of binding to said specific binding partner;
at least one detectable marker for detecting binding between said biomarker and said specific binding partner.

Diagnostic reagents that binds specifically to a biomarker include an antibody or a nucleic acid molecule specifically hybridizing under stringent conditions to said biomarker. The skilled person is well aware of the various methods of detecting mRNA of protein biomarkers by using specific binding partners.

All reference referred to herein are incorporated by reference herein in their entirety.

The present invention will now be further illustrated in the Experimental part described below.

### EXAMPLES

### Example 1

### Therapeutic Methods Relating to RIKEN cDNA 9430020K01 Gene Expression

Through genome wide analysis (using RNA microarray analysis) of mouse and zebrafish embryonic vessel development, we have identified 2000+ genes involved in arterial development. In specific, through genome wide microarray analysis of RNA expression profiles in Flk1+ cells during mouse development by Affymetrix chips, we have identified genes that specifically are upregulated during a time window during which vaculogenesis predominates. Using a trans-species approach, we further selected and verified expression of these candidate genes in the developing vascular tree in mice and zebrafish, and their upregulation during ischemia in adolescent mouse and pig models. Finally, induced expression of individual clones was verified by qPCR analysis in subsets of circulating PML of blood samples obtained from patients admitted with stable ischemic coronary artery disease and with acute coronary syndrome.

These newly identified regulatory genes in vascular development and repair are further studied by use of cell culture studies, mouse and zebrafish genomics and in several animal models of peripheral artery ischemia, myocardial ischemia, acute myocardial infarction, atherosclerosis development (including vulnerable plaque development), lymphangiogenesis, retinal angiopathy and tumor angiogenesis. Expression profiles of these vasculogenic genes are also studies in selected patients with cardiovascular disease using the proposed customized microarray technology, including patients with manifest peripheral artery disease, myocardial ischemia (w/wo appropriate anti-ischemic therapy), acute myocardial infarction, heart failure and patients that were treated with a percutaneous coronary intervention (balloon angioplasty, stenting).

A genome wide screen of various stages of vascular development during mouse embryogenesis identified known as well as complete unknown and undocumented clones, designated EST clones or RIKEN clones. Some of these candidate genes did not have an amphibian orthologue and were further selected by their vasculature specific expression by comparing gene expression levels in isolated human aorta and highly vascularized tissue as opposed to other irrelevant organs by qPCR. Expression of the RIKEN cDNA 9430020K01 gene was determined in different organs in mice. Expression as established by qPCR was high in highly vascularised tissues, such as lung, the aorta and the carotis artery in comparison to irrelevant organs such as kidney, liver, brain, eye, hart and skeletal muscles (see FIG. 1).

Ensuingly, endothelial specific expression was further analysed using endothelial primary cell lines, Human Umbilical Venous Endothelial Cells (HUVECs), and in vivo in the mouse. RIKEN cDNA 9430020K01 was up-regulated during vessel formation in the developing mouse and was exclusively expressed in the vascular network. In HUVECs cell culture, knockdown of RIKEN cDNA 9430020K01 remarkably inhibited cell proliferation, by a cell-cycle arrest in the G1-phase. Also, concomitantly, more apoptosis was identified in the RIKEN cDNA 9430020K01-silenced endothelial cells. In line, tube formation by HUVECs in a 2D-matrigel assay was significantly attenuated by RIKEN cDNA 9430020K01 knockdown, while migration of HUVECs was decreased. Next, gene function of RIKEN cDNA 9430020K01 was assessed in a murine retina model. Knockdown and overexpression of RIKEN cDNA 9430020K01 is induced by lentiviral infection of shRNA and cDNA expression vectors in the retina of C57bl/6 mouse pups directly after birth at day one. Immunohistological evaluation of the retina was performed to study vascular sprouting. Knockdown of RIKEN cDNA 9430020K01 impedes cell proliferation and increase apoptosis in EC in the retina in vivo.

This particular gene product was selected by the sheer potency of the identified clone to induce neovascularisation c.q. new vessel formation, equally, if not more potent, as compared to the most potent, hallmark neovascularisation regulatory gene (product) known to date, the VEGF family. This particular gene product is, to date, completely unknown, with no documented function or expression pattern.

### Set up of HUVECs

HUVECS were prepared in a quantity of one tube of HUVECs for two 15 cm plates by coating two 15 cm plates, each with 10 ml of 0.1% gelatine. Subsequently, gelatin was removed and dried for 30 minutes at room temperature. Then 20 ml EGM-2 medium was added. One tube of HUVECs was defrosted from the liquid nitrogen at 37° C. and 1 ml of the HUVECs was added to 3 ml EGM-2 medium in a 15 ml tube. 2 ml of the cell suspension was added on a 15 cm plate and placed 37° C., 5% CO₂. Medium was refreshed after 6 hours.

HUVECs were passaged by coating plates with 10 ml of 0.1% gelatine. Subsequently, the gelatine is removed and dried for 30 minutes at room temperature. Then 20 ml EGM-2 medium is added to the plates. The medium is subsequently removed and the plates are washed with PBS. The HUVECS are typsinized. Cells are suspended in EGM-2 medium and centrifuged for 5 minutes. The pallet is then resuspended in EGM-2 on plates and cultured at 37° C. in 5% CO₂.

To establish the effect of inhibition of the RIKEN cDNA 9430020K01 gene expression in HUVECs, three different experimental treatment regimes were established:
1. transfection with specific siRNAs against the RIKEN cDNA 9430020K01 gene,
2. transfection with non targeting siRNAs (sham) and a
3. negative control.

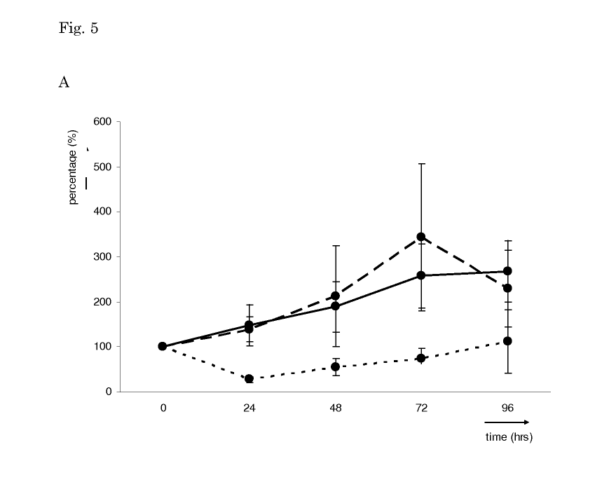

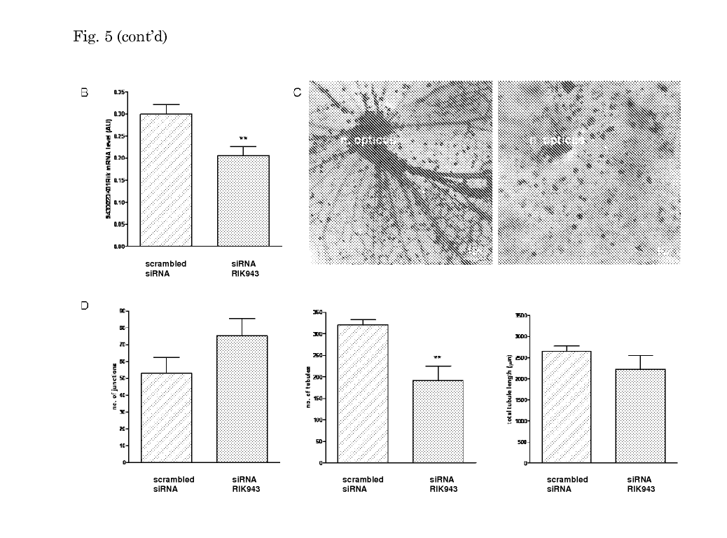

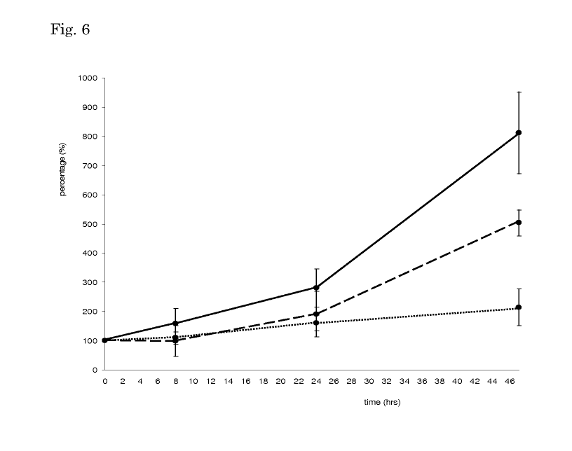

Regime 1 consisted of transfection with a mix of 3 different siRNAs, having SEQ ID NO:2 CGGGAAGAGGTGAGAGTAA, SEQ ID NO:3 CAGCATCACGCGAGGATAA and SEQ ID NO:4 GGATGAGAGTCCTGAGCTT at a concentration of 5-2.5-1 ng/ml. (See FIG. 5)

Samples of the HUVECS exposed to these regimes were taken at different timepoints up to 120 hrs after the first exposure.

Silencing of the targeted gene was verified by RT-QPCR in infected HUVECS, using forward primer having SEQ ID NO: 5 AGAGCTGGAGTGAGGAGCTG and reverse primer having SEQ ID NO: 6 TCCATTCCGTTGCTAACCTC, resulting in a product of 157 bp.

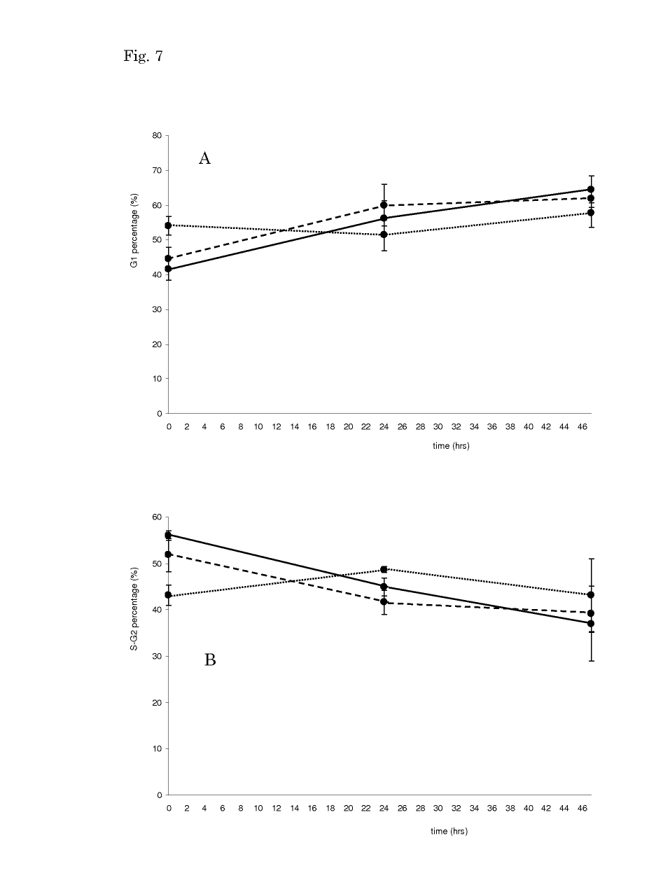

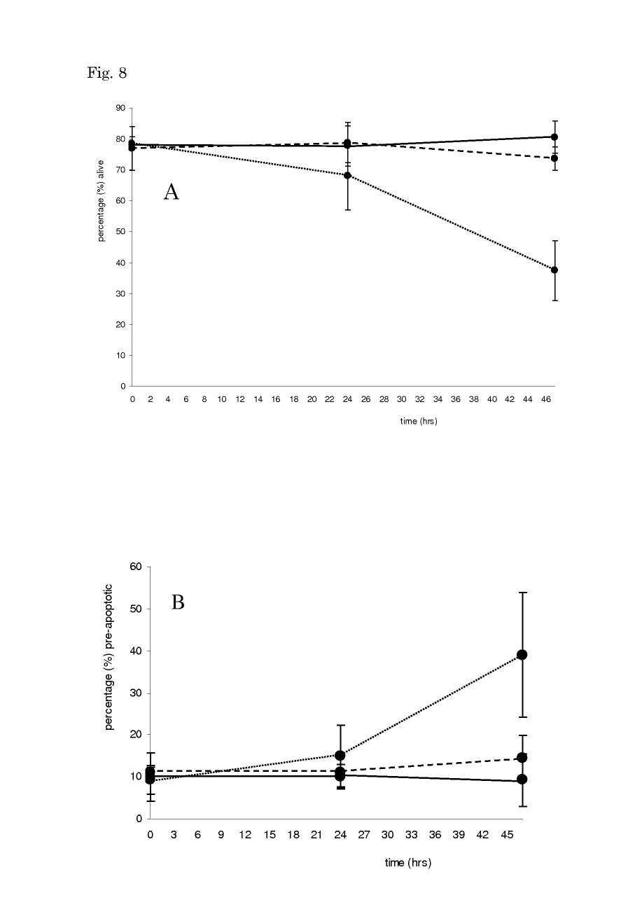

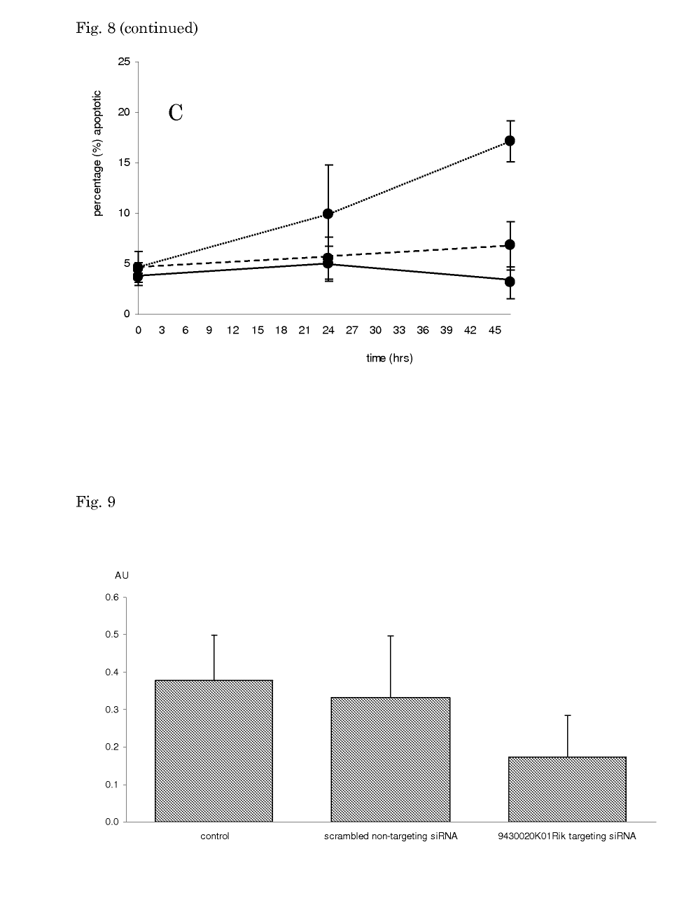

A proliferation assay was performed to count the number of dead cell using Tryptane blue at day 1-5 after the exposure of HUVECS to the three different regimes (see FIG. 8 A). In addition, the number of cells in G1 and G2 phase was established by FACS analysis using a Propidium iodide staining (see FIGS. 7 A and B).

The number of apoptotic cells was determined using the Annexin V-FITC Apoptosis Detection Kit I (BD Pharmingen™ cat. no. 556547, lot: 18354) (see FIG. 8 B and C).

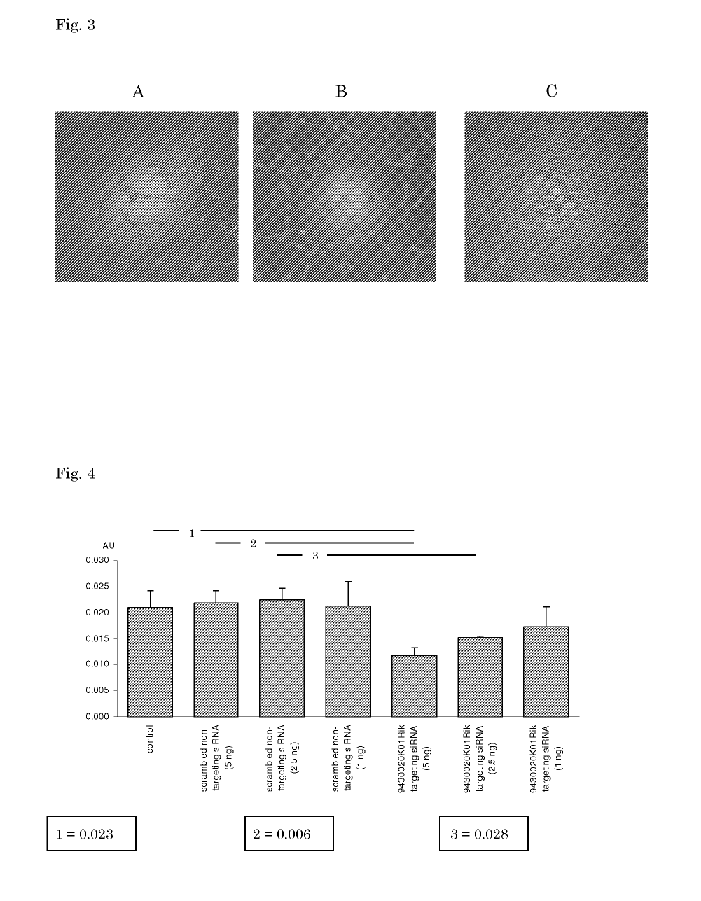

The effects of inhibition of the RIKEN cDNA 9430020K01 gene on the ability of HUVECS to form tubes were determined by means of a 2D matrigel (tube formation) assay as known in the art using a Calcein-AM staining (see FIG. 3).

### Example 2

### Therapeutic Methods Relating to Agtrl1/Apelin Expression

### Materials and Methods

All experiments were conducted in compliance with institutional (Erasmus University Medical Center, Rotterdam, The Netherlands) and national guidelines.

### Animals

Female C57BL/6J mice (age 8 weeks) were obtained from the Jackson Laboratory (Bar Harbor, Me., USA) and randomly assigned to an experimental group.

Apelin used in the in vivo models was derived from Sigma (Apelin-13 trifluoracetate salt; A6469-1 mg)

### Surgical Procedures

The mice (age 12 weeks) were weighed, sedated with 4% isoflurane, intubated and pressure-controlled ventilated with O₂—N₂O (1:2, vol/vol) containing ˜2.5% isoflurane for anesthesia. To test the effect of Apelin on EPC mobilization animals were treated with Apelin 400 ng/kg/min via a subcutaneously implanted osmotic minipump (Alzet, model 1003D, Durect Corp, The Netherlands) for the duration of the experiment. As a control PBS was infused. EPC recruitment was tested by injection of Apelin or PBS directly into the calf muscle or the left ventricle. Animals were sacrificed after 3 days and tissues were harvested for subsequent analysis.

MI was induced by permanent ligation of the left-anterior-descending-coronary-artery (LAD) with a 7-0 silk suture (B. Braun, Aesculap AG&CO.KG, Tuttlingen, Germany) using standard procedures. HLI was induced by permanent ligation of the femoral artery with a 6-0 silk suture (B. Braun). Sham animals underwent the operation without ischemia induction. To test the effect of Apelin after MI animals received an injection of Apelin, PBS or no injection directly after the induction of MI. Animals were sacrificed at 3 days or 2 weeks. At sacrifice, hemodynamic measurements were performed under anesthesia as described before. In brief, short and long axis view M-mode LV echocardiography (ALOKA, Prosound SSD-4000; Japan) was performed and LV diameters at end-diastole (LVEDD) and end-systole (LVESD) were measured, and fractional shortening calculated. Tissues were harvested for subsequent analysis. At the conclusion of each experiment, left (LVW) ventricular weight including septum, tibia-length (TL) and lung fluid weight were determined.

### QPCR and Protein Analysis

mRNA levels were determined from freshly isolated left ventricles and calf muscles. RNA was extracted using the RNeasy kit (Qiagen, The Netherlands). Quantity and quality were verified with the aid of the nanodrop and Agilent 2100 Bioanalyzer electrophoresis (Agilent Technologies, UK). Isolated RNA was reversed transcribed into cDNA (Iscript, Biorad, The Netherlands). QPCR was performed by realtime fluorescence assessment of sybrgreen signal in the iCycler iQ Detection system (Bio-Rad, the Netherlands). Apelin mRNA levels were analyzed and normalized for the housekeeping gene HPRT.

Protein levels were assessed from freshly isolated left ventricles, calf muscles and blood plasma. Forty milligrams of tissue was homogenized, boiled in 0.1 mol/l acetic acid for 10 min, and then centrifuged at 15000 rpm for 10 min, and the supernatant was used to quantify total protein concentration by the Bradford Assay. Equal amount of total protein (300 μg/ml) were used in the Apelin-12 EIA kit (Phoenix pharmaceutical, US), according to manufacturers prescription.

### Flow Cytometry

Cell analysis was performed on BM, blood, ventricle and skeletal muscle. Mononuclear cell suspensions of BM and blood were obtained by density centrifugation and subsequent washing in RPMI. Cells suspension of ventricles and skeletal muscle were isolated by homogenization and subsequent washing over a 40 mm cell strainer (Falcon). Isolated cells were stained with antibodies against Flk (Biotin conjugated, BD Biosciences, Belgium), cKit (APC-Cy7 conjugated, eBiosciences, UK), Sca-1 (APC conjugated, eBiosciences, UK) and Agtrl-1 (LifeSpan Biosciences, Seattle, US). Antibodies (Ter119, B220, CD11b, GR1, CD3, CD4 and CD8 all from eBiosciences) to exclude LIN+ (Ter119, B220, CD11b, GR1, CD3, CD4 and CD8, PE conjugated, Biosciences) cells were included as well. Cells were incubaded with the antibody mix for 15 min at 4° C. After washing, cells were incubated with goat anti rabbit Alexa 488 (Invitrogen) and streptavidin PE-Cy7 (eBiosciences) for 15 at 4° C. 7AAD (BD biosciences, Belgium) was added to assess cell death.

Flowcytometric analysis was performed with the use of a FACSCanto (BD biosciences, Belgium) and subsequent data analysis with analysis system (Flowjo).

### Histological Analysis

Left ventricles were embedded in paraffin. Paraffin embedded ventricles were longitudinally cut in 5 mm sections and stained with Masson's trichrome to determine infarct size. Capillary density was assessed by lectin staining. Briefly, sections were dewaxed, hydrated and blocked with 5% BSA for 15 min. Sections were washed in PBS and incubated for another 120 minutes with lectin from BS (HRP conjugated, Sigma). After washing in PBS, sections were treated with niDAB for 3 min. After another washing, sectioned were counterstained with nuclear fast red and coverslipped. Data analysis was performed using a commercial image analysis system (Impak C, Clemex Technologies, Canada)

### Statistical Analysis

Data were analyzed by using unpaired student's T-test or one-way ANOVA when appropriate. Significance was accepted when P<0.05. Data are presented as mean±SEM.

### Zebrafish Procedures (and Morpholino's)

### Zebrafish

Zebrafish were kept under standard husbandry conditions at the Hubrecht Institute. Transgenic lines used were Tg(kdr-l:GFP)^{s843} (Jin et al., 2005) and Tg(fli1:neGFP)^{y7} (Roman et al., 2002).

### Tilling of Agtrl1b

A library of randomly ENU mutagenized F₁-zebrafish was screened for mutations in the agtrl1b gene by DNA resequencing (Wienholds, 2002). After filtering for SNPs, 26 ENU-induced mutations were detected of which one led to a premature stop just after the first transmembrane domain (W54Stop). The identified F₁ carrier fish (agtrl1b^{hu4145}) was crossed into the desired transgenic backgrounds and bred for several generations (up to F₅) to remove any unwanted background mutations. Primers used for TILLING were:

Ag1-1A-1-5′-GACCTGAGCACATGACAAAC-3′,
Ag1-1A-2-5′-TGTAAAACGACGGCCAGTTGGACAACATGAC
TGCTGAC-3′,
Ag1-1A-3-5′-AGGAAACAGCTATGACCATCAGATGGCTGTCA
GAGAGG-3′
and
Ag1-1A-4-5′-ATACACTGTGGTGCGGAAG-3′.

### Morpholino Injections

Morpholinos (MOs) against silent heart (SiH) (5′-CATGTTTGCTCTGATCTGACACGCA-3′) (Sehnert et al., 2002) were obtained from Gene Tools (Gene Tools, LLC, Philomath, USA) and diluted in water containing 0.2% phenol red. One cell stage embryos were injected with 1 ng of MO with a maximum volume of 1 nl.

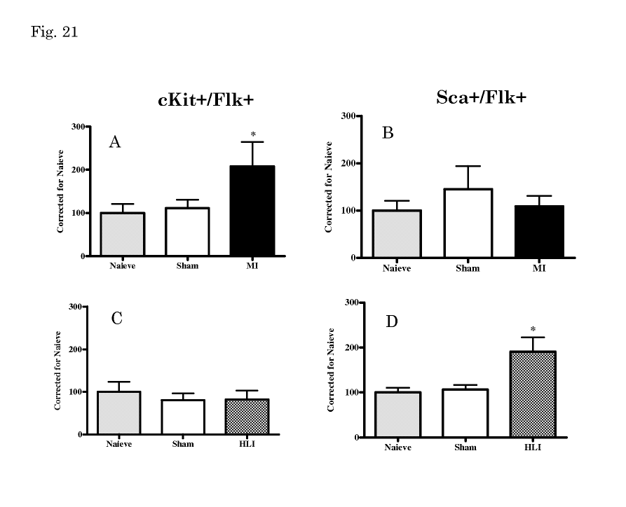

The results of the study described in this Example are presented in FIGS. 21-26.

In FIG. 21 it can be seen that following an acute myocardial infarction, specifically cKit+/Flk1+ endothelial progenitor cells are recruited in the circulation, whereas following hind limb ischemia in particular Sca1+/Flk1+ cells are recruited into the circulation.

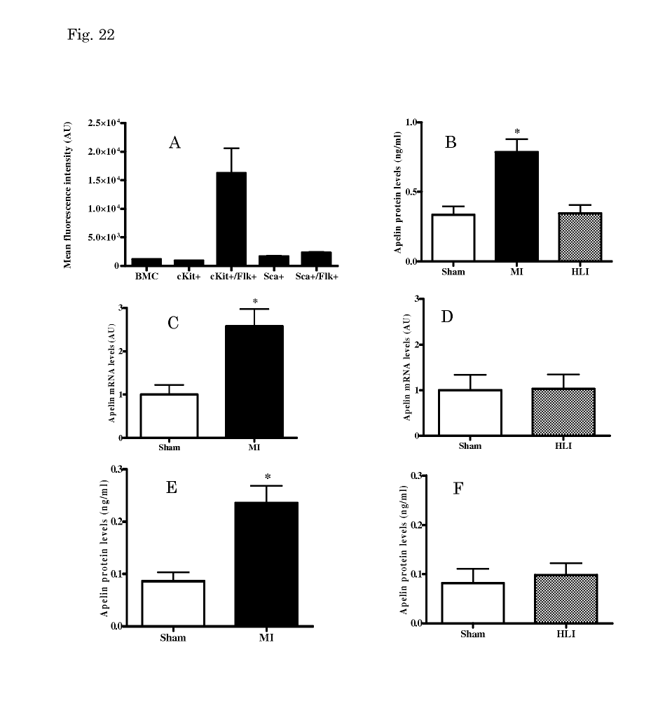

In FIG. 22 it can be seen that after ischemia, Agtrl1 is specifically expressed in cKit+/Flk1+ endothelial progenitor cells (involved in the (repair) response following an acute myocardium; infarction), and that following an acute myocardial infarction, apelin (the ligand of Agtrl1) is regulated in the circulation, as well as in the myocardium on protein and mRNA level.

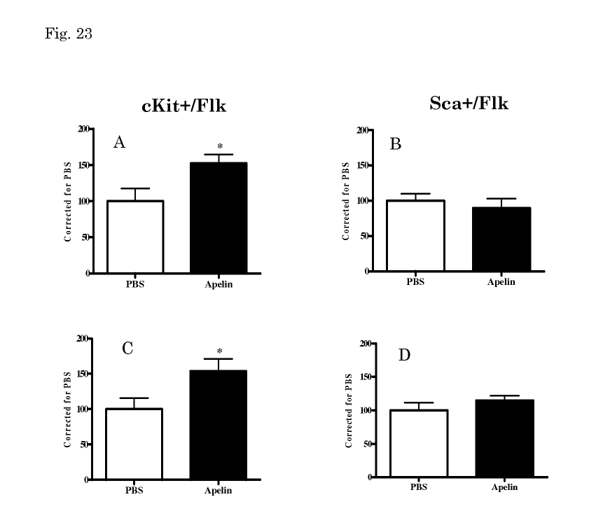

Systemic infusion of apelin increases the number of cKit+/Flk+ endothelial progenitor cells in bone marrow and blood, whereas apelin infusion does not change the number of Sca+/Flk+ cells in these tissues (FIG. 23). Hence, systemic infusion of apelin specifically stimulates the number of cKit+/Flk1+ in the bone marrow as well as in the circulation.

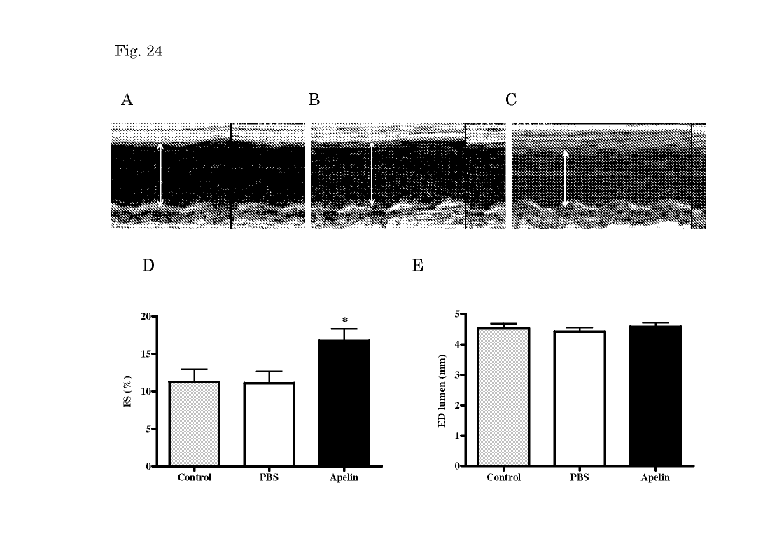

Apelin treatment improved the Fractional Shortening of the left ventricle and resulted in better contraction during systole (FIG. 24). Therefore, it is concluded that systemic treatment with apelin will improve global cardiac function following an acute myocardial infarction.

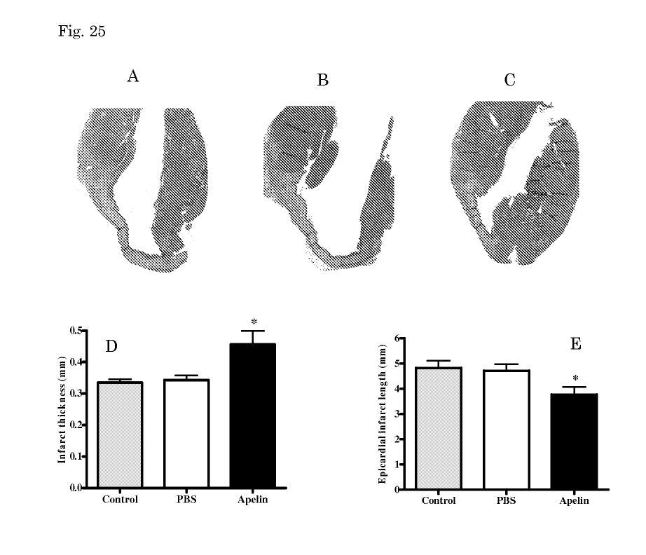

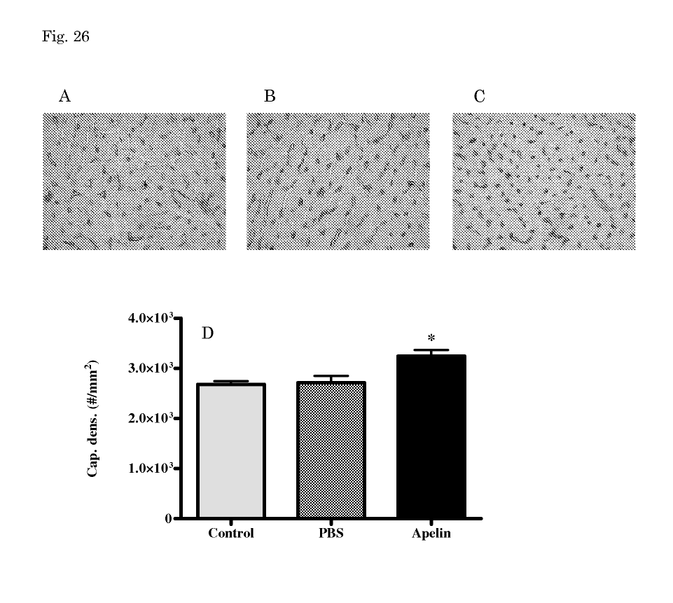

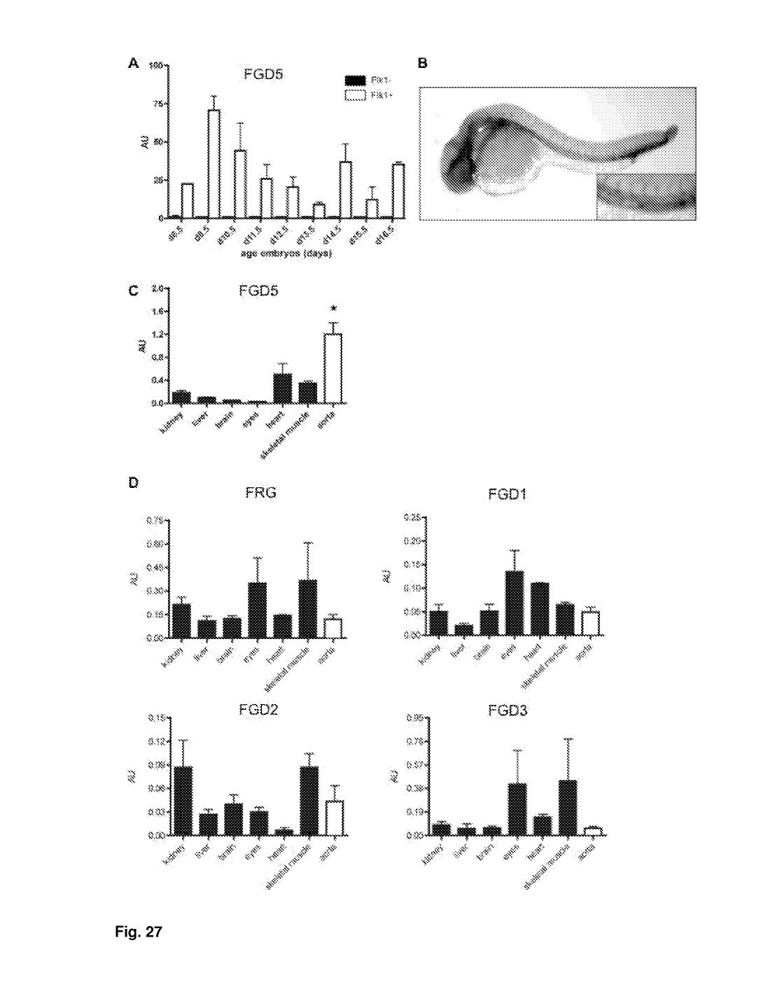

Studies on the scar formation after MI and apelin treatment indicated that apelin treatment increases myocardial thickness of the border zone and diminishes infarct length (FIG. 25), and apelin infusion is therefore indicated to reduce infarct size after myocardial infarction. Furthermore, apelin infusion stimulates new vessel formation following a myocardial infarction (FIG. 26). The above therapeutic effects of apelin are expressly identified as aspects of the present invention.

### Example 3

### Therapeutic Methods Relating to FGD5 Expression

### Materials and Methods

Isolation of Flk⁺ and Flk⁻ Cells from Mouse Embryos

10-15 dpc FVB/N mouse embryos were harvested digested to obtain single cell suspensions in PBS/10%FCS/0.12% collagenase type I (Sigma C-0130, The Netherlands). Single cell suspensions were labelled with 1:50 Flk-1 PE mouse antibody (BD bioscience, The Netherlands) and 1:100 Hoechst, and sorted for >90% purity for Flk+/Hoechst− cells using a FACS Diva cell-sorter. Cells were dissolved in RLT buffer and processed for qPCR analysis.

### Quantitative PCR and Western Blot Analysis

RNA isolated from various tissues form C57bl/6 mice or primary cultures using the RNAeasy kit (Qiagen, The Netherlands) was checked for quality and quantity by capillary electrophoresis (Agilent 2100 Bioanalyzer, Agilent Technologies, The Netherlands), and reversed transcribed into cDNA. qPCR reactions were performed by real-time fluorescence assessment of the sybergreen signal using the iCycler iQ Detection System (Bio-Rad, The Netherlands). qPCR analysis was performed for mouse FRG, FGD1-6, eNOS, and CD31, and for human VEGFR1/2, Notch1/4, DLL4, jagged1, ephrin B2/B4, NRP1/2, p53 and Hey1. Target gene mRNA expression levels are reported relative to the housekeeping genes, hypoxanthine guanine phosphoribosyl transferase (Hprt1) in murine samples, and beta actin in the human samples (primer sequences are provided in Table 1 and 2). For western blot analysis, cell culture and tissue samples were lysed in NP40 buffer, and analysed using SDS-PAGE western blots and 1:1000 rat anti-p21^{CIP1}, anti-p53 antibody (Abcam, UK), 1:500 rabbit anti-cdc42, anti-Rac1, and anti-RhoA antibody (Abcam, UK), and 1:500 mouse anti-FGD5 antibody (Bioscience, The Netherlands). Protein bands were visualized using the Li-Cor detection system (Westburg, The Netherlands), as previously described (Cheng et al. 2009. Circulation 119, 3017-3027).

TABLE 1

| Primers | | |
|---|---|---|
| (human) | Forward seq. | Reverse seq. |
| Notch1 | AGAGACTCCTGCTTCAACG | CACACCAGTGCACAAGGTTC |
| Notch4 | CACGTGAACCCATGTGAGTC | CACAGTGGAATCCTCCAGGT |
| DLL4 | GCATCCGTGTTTCCAAAAGT | AAGGGCCTAGACCAGAGAGC |
| Hey1 | TCGGCTCTAGGTTCCATGTC | AGCAGATCCCTGCTTCTCAA |
| Vegfr1 | ATTTGTGATTTTGGCCTTGC | CAGGCTCATGAACTTGAAAGC |
| Vegfr2 | AGCGATGGCCTCTTCTGTAA | ACACGACTCCATGTTGGTCA |
| Nrp1 | GAAGCACCGAGAGAACAAGG | CTCGGGGTAGATCCTGATGA |
| Nrp2 | TCCACTGCTGACAAGGTTTG | ACTGGGGCTCCAGAGGTATT |
| EphrinB2 | TCCATGGGTAATCCGTTCAT | TCAGCAAAACCAAAGTGCTG |
| EphB4 | GGTCTACATCGACCCCTTCA | TCTTGATTGCCACACAGCTC |
| VegfA | GGGCAGAATCATCACGAAGT | ATCTGCATGGTGATGTTGGA |
| Jagged1 | GACTCATCAGCCGTGTCTCA | TGGGGAACACTCACACTCAA |
| GAPDH | AATCCCATCACCATCTTCCA | TGGACTCCACGACGTACTCA |
| FGD5 | GAGGGCAGCAGTGAAGTAGG | GTGTGTGTCCGGATGAGTTG |

TABLE 2

| Primers | | |
|---|---|---|
| (murine) | Forward seq. | Reverse seq. |
| Hprt | CAGCCAACACTGCTGAAACA | TCAGGAGAGAAAGATGT |
| | | GATTG |
| FGD1 | TGTGATTGTTGCCTCGGA | TAGGCACCTTCTCACCAT |
| FGD2 | CAAGGAGGCCTTGAAGTC | CACGACCCTCTTCTCCTC |
| FGD3 | ATCCCCAACAGAGACAGT | AGGCCTAAACAGGGTGAC |
| FGD4 | GACTCCCCAGACTGGAAT | CGCTCTTGTGCTGATGTG |
| FGD5 | CTGCGTTAAGTTTCTGTGTG | CTCTCTTTCTCCTTTACC |
| | | TCTC |
| FGD6 | GCAGTCTTCCTGCCTCAA | ATGGGTTACGCAAGTTCC |
| FRG | TCAAACTCCACCCTGAGG | GAAGACAGCCTTTGCCTT |
| eNOS | GAGGACATTTTCGGACTCAC | GACTGTCAGGAACCAGGT |
| CD31 | TGCTCTCGAAGCCCAGTA | TGTGAATGTTGCTGGGTC |

### Histological Analysis

Mouse ventricle, and aorta/carotid arteries, were embedded in OCT (Sakura Finetek, The Netherlands) and snap-frozen in liquid nitrogen. 5 μm cryosections were double stained using Alexa-fluor coupled isolectin IB4 (1:500, Invitrogen, The Netherlands) and 1:100 mouse anti-FGD5 (Bioscience, The Netherlands), followed by FITC/rhodamin-labeled anti-mouse IgG antibody detection (1:500, Invitrogen, The Netherlands), and goat FAB against mouse IgG (H+L) for blocking (Jackson Immunoresearch Labs, USA).

### Primary Cell Culture Conditions

Primary human umbilical vein endothelial cells (HUVECs, Lonza, The Netherlands) in EGM2 medium (EBM2 medium supplemented with bullet kit and 2% FCS) with penicillin/streptomycin (Lonza, The Netherlands) were cultured on gelatin-coated plates at 37° C./5%CO₂. Only cell cultures of passages 3-5 were used throughout the experiments. For immuno-histological staining, HUVECs were grown on coverslips and fixed in ice-cold aceton for 5 minutes, followed by permeabilization by 0.1% tritonX/PBS, and stained by 1:100 mouse anti-FGD5 (Bioscience, The Netherlands) followed by TSA-amplification of the signal (Roche, The Netherlands). This is followed by incubation with 1:500 rabbit anti-cdc42 (Abcam, UK) or rat anti-Zyxin (1:500, Abcam, UK) and detection using 1:500 FITC labeled anti-rabbit/rat IgG antibody (Invitrogen, The Netherlands). Coverslips were mounted using Vectashield/DAPI (Brunschwig, The Netherlands) and imaged by fluorescence microscopy (Carl Zeiss Inc., The Netherlands).

Gene Transfer and siRNA Knockdown in Cell Culture

The pAd/CMV/V5 gateway commercial system (Invitrogen, The Netherlands) was used to generate the required recombinant adenoviral vector according to the manufacturer's recommendations and was transfected into 293 cells for adenoviral particle assembly. Viral particles were harvested from cell lysates and the viral titer was determined by viral plaque assays. ΔE1/E3-sham adenovirus was used as a control adenovirus (Sham Ad) in all studies. Sub-confluent HUVEC-cultures were infected in EGM2/0.2%FCS for 2 hours at 37° C./5%CO2 (moi 100), resulting in >90% transfection efficiency in HUVECs at 48 hours (Invitrogen, The Netherlands). Targeted knockdown of genes was achieved by transfer of a mix of 4 siRNAs sequences directed against the mRNA of each gene (Smartpool, Dharmacon, The Netherlands) in 50-60% sub-confluent HUVEC cultures, 3 days prior inclusion in experiments. As a control, cells were transfected with a mix of 4 scrambled non-targeting siRNAs (Dharmacon, The Netherlands). siRNA transfection efficiency of >80% was achieved at 72 hours, as validated by FITC labelled siRNA (siglow, Dharmacon, The Netherlands). Adequate overexpression or knockdown was validated by qPCR and western blot analysis at 2 and 3 days post transfection.

### In Vitro and Ex Vivo Angiogenesis Assays

Tube-formation assay: HUVECs were plated at a density of 3×10⁴ cells/ml in 200 μl EGM2 medium in a 96-well plate on serum-reduced Matrigel (BD Biosciences, The Netherlands) and incubated for 24 hours. Viable cells were visualized by Calcein-AM uptake according to the manufacturer's protocol (BD Biosciences, The Netherlands) and fluorescence microscopy. Coated bead assay: Trypsinized single cell suspensions of HUVECs (24 hours post transfection) were co-incubated for 4 hours with Cytodex microcarrier beads (Sigma, The Netherlands). At a ratio of 400 cells per bead in EGM2 medium, as described previously (Sainson et al. 2008. Blood 111, 4997-5007). HUVECs coated micro-beads were transferred to a 6-well plate and incubated overnight, followed by 2 washes to eliminate unattached cells. Coated micro-beads were resuspended in 2.5% human fibrinogen/ml EGM2 (Calbiochem, The Netherlands) supplemented with 1 μg thrombin (R&D Systems, UK), and plated in 12-well plate. Coagulated fibrinogen gels were covered by 1 ml EGM2 medium with 20 ng/ml bFGF, before visualization by rhodamin-phalloidin staining (Invitrogen, The Netherlands) or tuloidine blue staining at day 6. Ex vivo analysis of endothelial sprouting: Aortas of C57bl/6 mice were harvested, cut into 2 mm thick segments, and placed in serum-reduced Matrigel in a 96-well plate (BD Biosciences, The Netherlands). Imbedded aortic segments were then incubated with 200 μl EGM2 medium per well for 4 days before evaluation by phase-contrast microscopy. Quantification of the tube-formation assay: Angiosys analysis software (Angiosystems, UK) was used to determine mean and total tube length, and the number of tubes and junctions. Quantification of the coated-bead and aortic ring assay: Data analysis was performed using a commercial image analysis system (Impak C, Clemex Technologies, Canada).

### Mouse Retinal Angiogenesis Model

3 days old C57bl/6 pups were anesthetized by placement on ice. 0.5 μl Ad-FGD5 (5×10⁷ pfu) was intra-vitreal injected in the left and 0.5 μl sham adenovirus (5×10⁷ pfu) in the right eye using a 33-gauge needle. For FGD5 silencing experiments, 100 nmol accell siRNA targeting murine FGD5 was intra-vitreal injected in the left, and 100 nmol accell scrambled non-tageting siRNA in the right eye (Dharmacon, the Netherlands). At day 3, pups were sacrificed, and retinas were stained with rhodamin/FITC isolectin IB4 (1:200), rabbit anti-NG2 antibody (1:200, Millipore, The Netherlands), or rat anti-CD31 antibody (1:500, R&D Systems, The Netherlands) followed by Alexafluor-labelled anti-rabbit/mouse secondary antibodies (1:500, Invitrogen, The Netherlands). Adequate transgene expression was validated by qPCR analysis. For flow cytometric assessment of the retinal ECs, the retina was homogenized in 0.12% collagenase type I (Sigma C-0130, The Netherlands) in PBS/10%FCS for 15 minutes, filtered through a 3 micron mesh (BD Biosciences, The Netherlands), and stained with 1:50 PE-labelled mouse anti-Flk1 antibody, followed by Annexin V and PI staining. The percentage apoptotic or dead cells in the Flk1+ population, was quantified by flow cytometry (FACScanto, BD Biosciences, The Netherlands).

### In Vivo Coated-Bead Assay

Mature SCID mice (age 10-15 weeks) were injected subcutaneously with 700 beads in 300 μl Matrigel (400 HUVECs per bead), supplemented with 2.5 ng/ml fibrinogen and 20 ng/ml human bFGF. For each animal, 1 sham virus and 1 FGD5 overexpressing EC coated-bead plug was implanted in the flank. At day 8, solidified Matrigel plugs were retrieved, washed, fixed in 4% PFA/PBS, and imbedded in OCT (Sakura Finetek, The Netherlands). 5 μm cryo-sections were stained by Hematoxylin/Eosin staining, or by rat anti-CD31 (1:500) and rhodamin-labeled anti-rat IgG secondary antibody (1:500, R&D Systems, The Netherlands). For quantification of the % nuclei and % CD31+ cells, data analysis was performed using a commercial image analysis system (Impak C, Clemex Technologies, Canada).

### Cell Proliferation, Apoptosis and Cell Cycle Analysis

Transfected HUVECs were synchronized in G0/G1 phase by serum deprivation in EGM2/0.2%FCS for 12 hours. For cell growth assessment, cells were harvested and quantified using a hematocytometer (trypane blue negative) at different time points. 3-(4,5-dimethylthiazol-2-yl)-2,5-diphenvyl tetrasodium bromide (MTT) uptake experiments for cell metabolism assessment was performed according to manufacturer's protocol (ATCC, US). Cell-cycle analysis: Cells were harvested at 0.4 and 12 hours post activation, and fixed in 70% ethanol/PBS for 15 min on ice, stained with propidium iodide (PI 1:300), and analyzed by flow cytometry. Apoptosis analysis: Cells were harvested at 0, 4 and 12 hours post activation, stained for Annexin V and PI signals using an Annexin V apoptosis detection kit (BD Biosciences, The Netherlands), followed by analysis of the samples by flow cytometry (FACScanto, BD Biosciences, The Netherlands).

### Small G Protein Activation Assay and FGD5-Protein Complex Co-Immunoprecipitation

GTP-RhoA, Rac1, and cdc42 activation levels were measured using the G-lisa detection system (Tebu-Bio, The Netherlands). For FGD5-protein complex co-immunoprecipitation, magnetic beads (Dynabeads, Invitrogen, The Netherlands) were coated and cross-linked with 2.5 μg anti-FGD5 antibody (Bioscience, The Netherlands) before immunoprecipitation overnight at 4° C. with protein cell lysates of transfected HUVECs (50 μg total protein of a volume of 100 μl incubation buffer). Beads were washed and the protein samples were eluted with eluation buffer (Invitrogen, The Netherlands) before analysis by western blot.

### Results

Genome-wide expression profiling of RNA transcripts isolated from Flk1+ endothelial angioblasts during murine development, identified FYVE, RhoGEF and PH domain-containing 5 (FGD5) as one of the −2000 genes that was specifically upregulated in FLk1+ angioblasts during early embryonic development. The FGD-family members of Rho guanine-nucleotide exchange factors (GEFs) includes FGD1-6 as well as the FGD1-related cdc42-GEF (GRF), and share the Dbl homology (DH), FYVE, and pleckstrin homology (PH) domains. The presence of the DH domain in FGD5 predicts binding and activation of small GTPases including cdc42, Rac1, and RhoA. Indeed FGD1, FGD4 and FRG have also been shown to exert cdc42 regulatory activity by converting inactive GDP-bound cdc42 into active GTP-bound cdc42 in fibroblasts and epithelial cells in vitro, modulating actin cytoskeleton assembly, filopodia formation, and JNK pathway activation via cdc42. However, the basic function of FGD5 is unkown. Here we sought to define the potential regulatory role and molecular function of FGD5 in vascular development.

### FGD5 is Specifically Expressed in Endothelial Precursor Cells During Murine and Zebrafish Development, and in Fully Differentiated ECS.

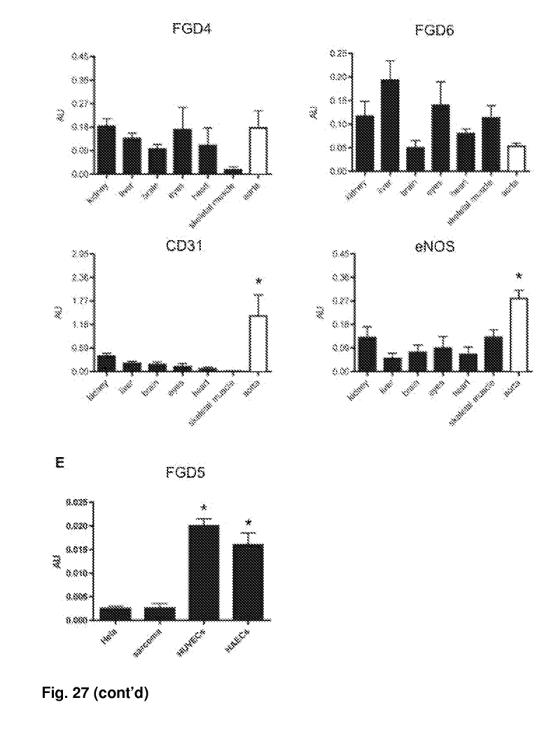

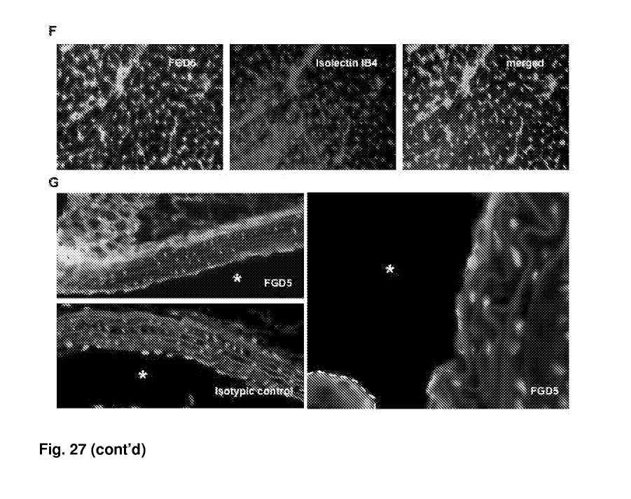

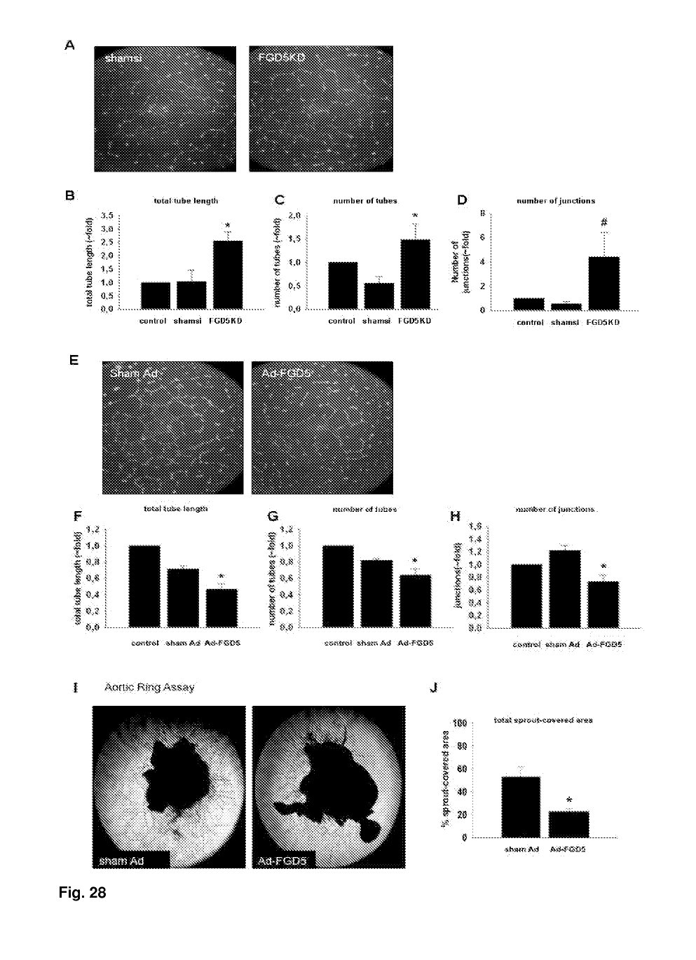

FGD5 was specifically expressed in the Flk1+ angioblast population during embryonic development in mice as shown by micro-array analysis and subsequent qPCR validation. FGD5 mRNA was predominantly expressed in Flk1+ precursor cells from 9 dpc until 15 dpc, when the majority of the vascular structures are established (FIG. 27A). Specific vascular expression of FGD5 in the developing vascular tree was also detected in zebrafish larvae by whole mount in situ hybridization: FGD5 mRNA expression was localized in the dorsal aorta, the posterior cardinal vein and in the intersegmental arteries (FIG. 27B). Using qPCR analysis, FGD5 expression in mature C57/bl6 mice, was predominantly expressed in the large blood vessels, as compared to heart, skeletal muscle, kidney, liver, eye and brain tissues (FIG. 27C), closely mimicking the expression profile of endothelial-specific markers, including eNOS and CD31, whereas the other FGD-family members showed a more ubiquitous expression pattern (FIG. 27D). In vitro, FGD5 was specifically expressed in primary human arterial and venous ECs (HUVECs and HAECs) as compared to non-relevant cells (Hela and sarcoma cell, FIG. 27E), underlining the endothelial specific expression in different species and developmental stages. Immuno-histological studies demonstrated FGD5 selective expression in ECs in the microvasculature of the myocardium and in the endothelial lining of large blood vessels, including the aorta (FIG. 27F,G). These findings were further confirmed by analysis of public gene expression databases (NCBI, Gene Expression Omnibus). These data distinguish FGD5 as the predominant isoform of the FGD-family specifically expressed in ECs during embryonic vascular development and in adult vasculature.

### FGD5 Inhibits Angiogenesis in Human Endothelial Cell Culture.

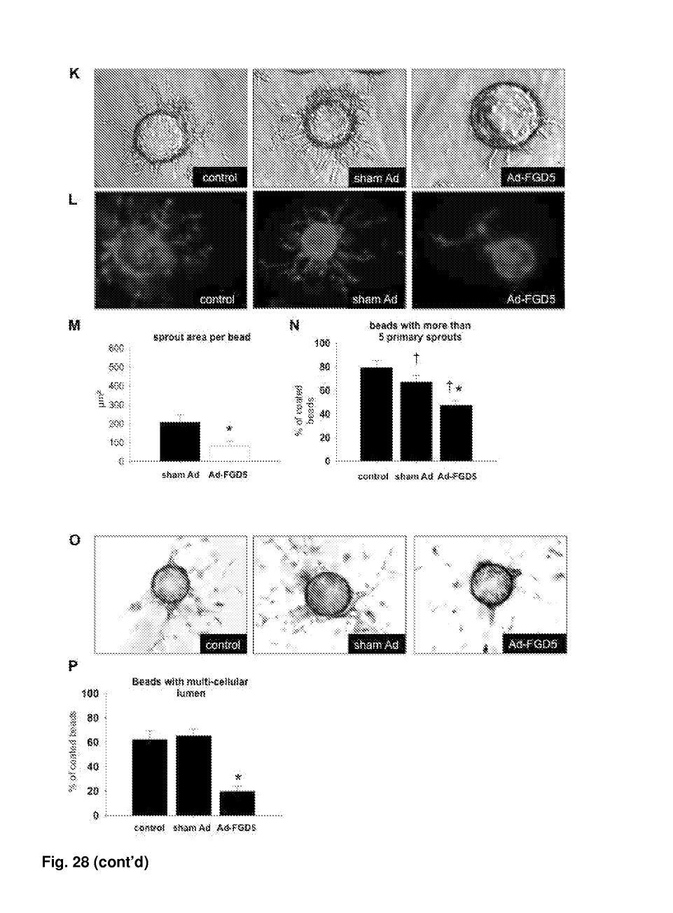

FGD5 function in cultured ECs (HUVECs) was assessed by loss and gain-of-function studies by transfection of FGD5 targeting siRNA, or by recombinant adenoviruses encoding for human FGD5 cDNA. In a 2D-matrigel tubulogenesis assay, siRNA-mediated silencing of FGD5 resulted in an increase of tube length, number of branches, and junctions of growth sprouts (FIG. 28 A-D), whereas FGD5 overexpression induced attenuated growth of tubes, branches and junctions (FIG. 28 E-H) suggestive of a role for FGD5 in oxygenation induced pruning of excessive capillaries during natural vascular remodelling. Similarly, in an ex vivo murine aortic ring assay, adenoviral-mediated overexpression of FGD5 significantly reduced microvasculature outgrowth as compared to sham virus transduced rings (FIG. 28 I, J). In addition, in an established model using HUVECs coated cytodex beads for angiogenesis, FGD5 overexpression inhibited sprouting of capillary-like structures as compared to sham virus transfected controls (FIG. 28 K-P). DAPI-staining was used to verify comparable number of attached viable cells to the beads at initiation of the assay.

### FGD5 Diminishes Angiogenesis In Vivo.

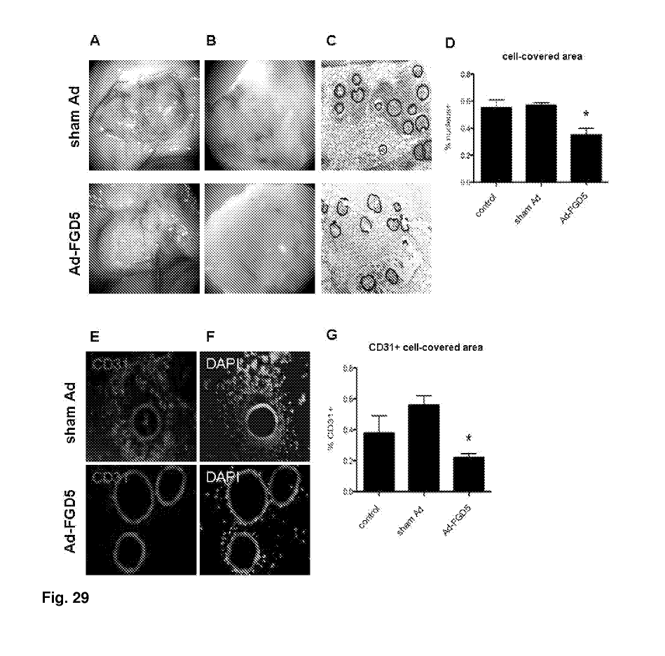

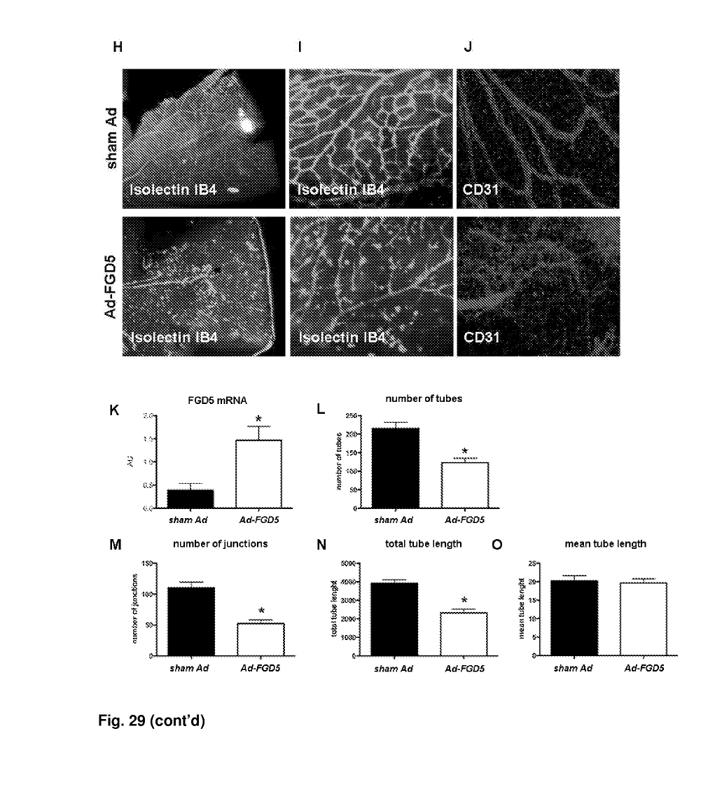

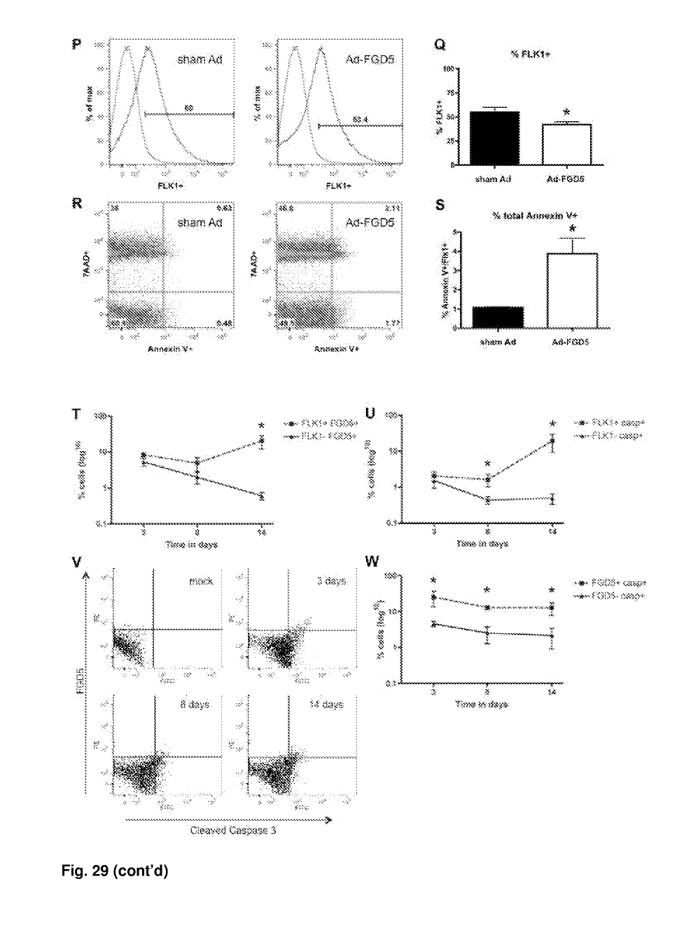

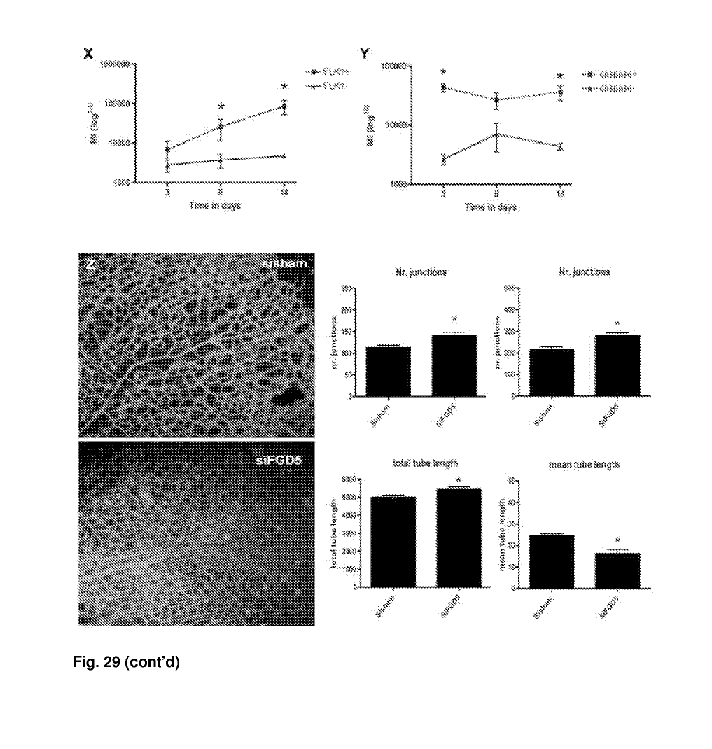

In order to extend the evaluation of the role of FGD5 in angiogenesis in vivo, beads coated with Ad-FGD5 or sham adenovirus transduced HUVECs were suspended in matrigel and injected s.c. in immunodeficient SCID mice. Matrigel plugs harvested at day 8 post transplantation revealed an attenuation in plug vascularization in the Ad-FGD5 plugs, whereas the sham plugs showed an extensive neo-capillary network (FIG. 29 A,B), and an increase in peri-bead outgrowth of CD31+ cells (FIG. 29 C-G). Similarly, during postnatal retinal angiogenesis, FGD5 function was assessed using an adenoviral vector encoding full-length murine FGD5 (mFGD5) cDNA (FIG. 29 K). Adenoviral mFGD5 overexpression by intravitreal injection (0.5×10⁷ pfu) reduced the retinal vascular network in the developing murine eye (FIG. 29 H), caused by truncation of vascular structures in the superficial vasculature (FIG. 29 I,J): Computer-assisted image analysis identified a decrease in total vascular tube length, number of junctions and tubes of the neo-capillaries (FIG. 29 L-O), implying that mFGD5 overexpression significantly reduces vascular density. mFGD5 overexpression in the murine retinal bed was associated with a decrease in the % Flk1+ cells (P,Q) and an increase in apoptosis in the FLK1+ cell population (R,S) as demonstrated by flow cytometric analysis of transfected retinas after a single intravitreal injection of Ad-mFGD5 or sham adenovirus. These data indicate that FGD5 plays a significant role in the development of the retinal vascular network, pointing towards either an inhibitory function on vascular growth or a pruning function in removing surplus neo capillaries during vascular remodelling. Assessment of the expression level of endogenous FGD5 during retinal vasculature development by flowcytometric analysis, showed a significant increase in the percentage of FLK1+ (endothelial) cells in the FGD5+ cell population, which coincided with a an increase in FLK1+ cells in the cleaved caspase 3+ (apoptotic) cell fraction (FIG. 29 T,U). More importantly, in the FLK1+ cell population, the cleaved caspase 3 signal was associated with FGD5 expression at all the different timepoints assessed (FIG. 29 R,S). In addition, a high mean of intensity (MI) level of FGD5 was associated with the FLk1+ and cleaved caspase 3+ population (FIG. 29 V,W). Together these data provide evidence that endogenous high FGD5 levels are associated with EC apoptosis. SiRNA mediated knock down of FGD5 further validated our findings, as FGD5 inhibition resulted in the persistence of excessive branches and tube structures that were normally removed during the vascular remodelling process (FIG. 29 V-Z).

### FGD5 Inhibits Proliferation and Induces Apoptosis in Endothelial Cells.

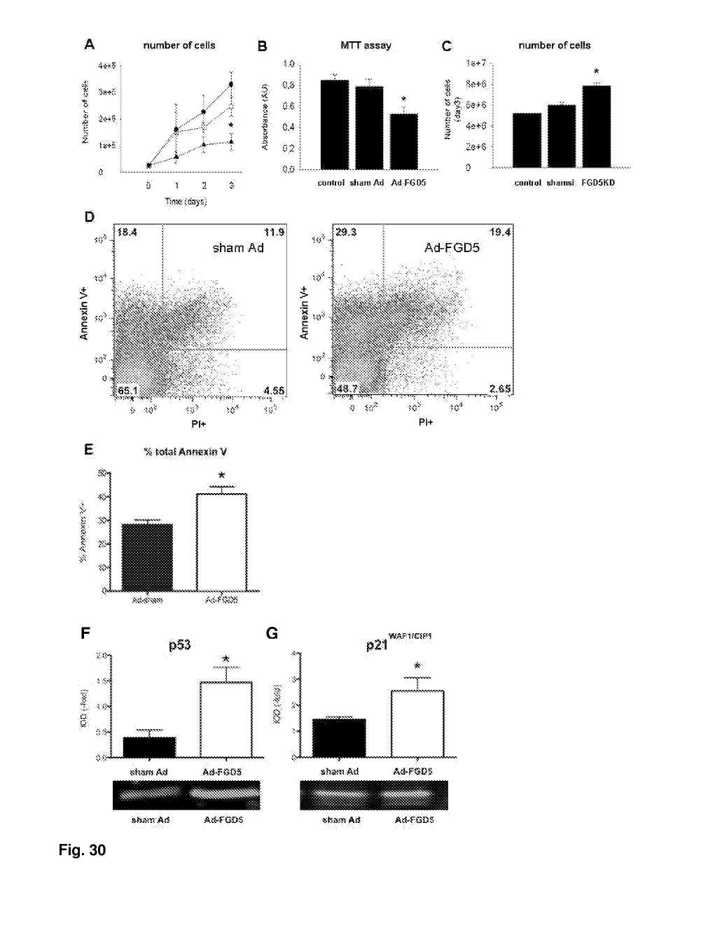

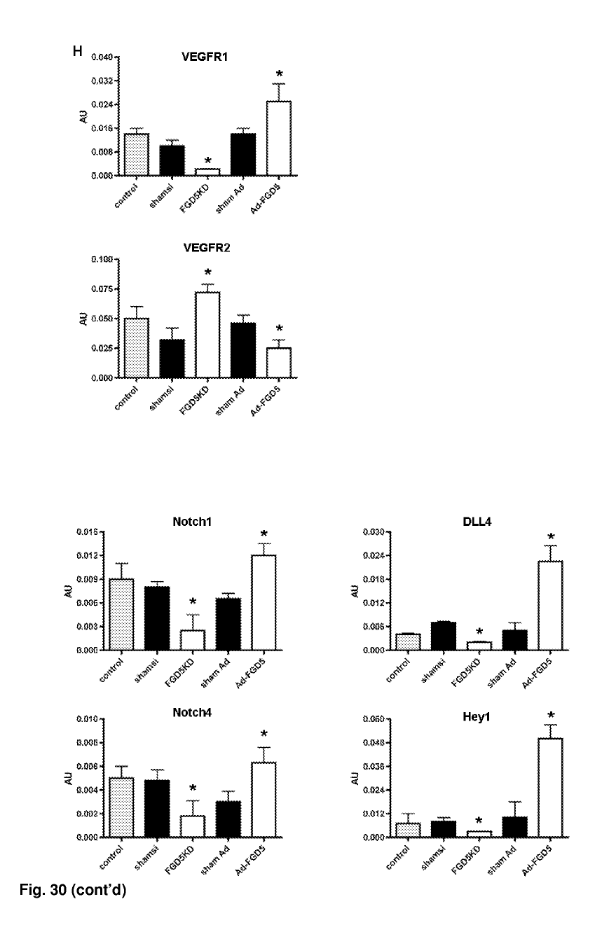

The observed anti-angiogenic effects of FGD5 could be attributed to changes in EC-proliferation rate. FGD5-expressing HUVECs showed a reduced cell growth rate in vitro as compared to sham virus transfected controls (FIG. 30 A, B). In contrast, siRNA-mediated FGD5 knockdown HUVECs showed an increase in cell proliferation compared to scrambled siRNA controls (FIG. 30 C). Next, we analyzed using flow cytometric evaluation of AnnexinV/propidium iodide the effect of FGD5 on apoptosis. FGD5 overexpression promoted apoptosis in ECs (FIG. 30 D, E), and was associated with upregulation of p53 and p21^{CIP1} protein levels (FIG. 30 F, G). These data imply that FGD5 could be involved in p53 dependent apoptosis and p21^{CIP1} associated cell cycle arrest resulting in vascular pruning.

### FGD5-Induced Apoptosis is Mediated Via Hey1-p53 Regulatory Pathway.

To further elucidate the downstream signalling cascade involved in FGD5-mediated vascular pruning, the gene expression profiles for well-defined angiogenic modulators were determined by qPCR. FGD5 knockdown or overexpression in primary EC culture did not change the mRNA expression levels of VEGFA, Jagged1, NRP1, NRP2, ephrinB2, and ephB4. In contrast, knockdown of FGD5 inhibited VEGFR1/Flt1, and promoted VEGFR2/KDR/Flk1 expression (pro-angiogenic state), whereas upon FGD5 overexpression, VEGFR2 was decreased in favour of VEGFR1 mRNA levels (anti-angiogenic state, FIG. 30 H). In addition, FGD5 knockdown was associated with the upregulation of Notch pathway genes, including Notch 1 and 4, and DLL4 (FIG. 30 H). Interestingly, Hey1, one of the downstream transcriptional regulators of the Notch signalling pathway, was markedly downregulated by knockdown of FGD5 (FIG. 30 H).

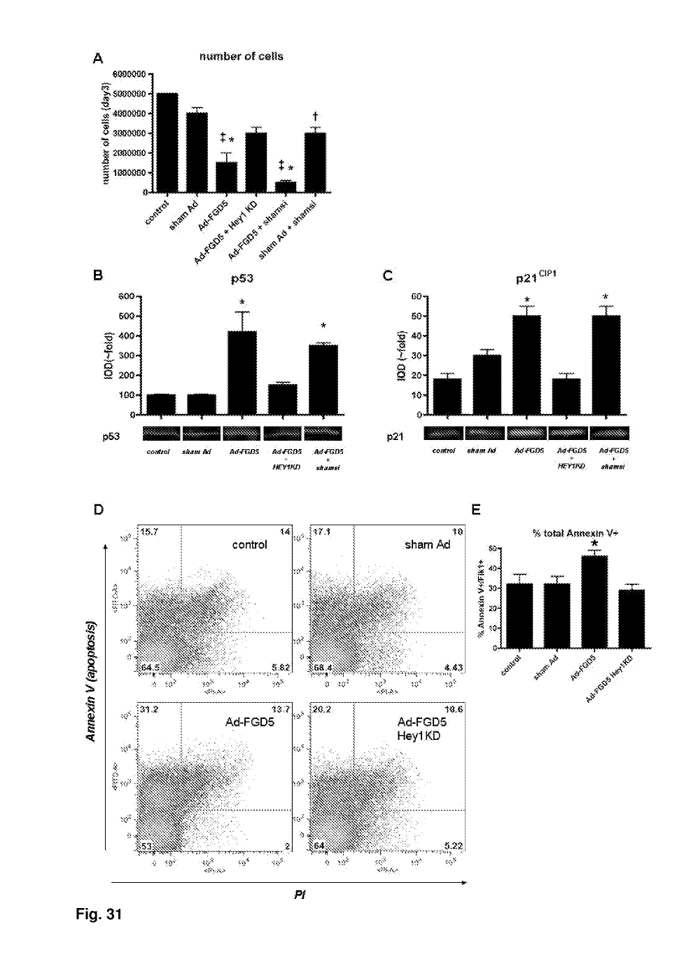

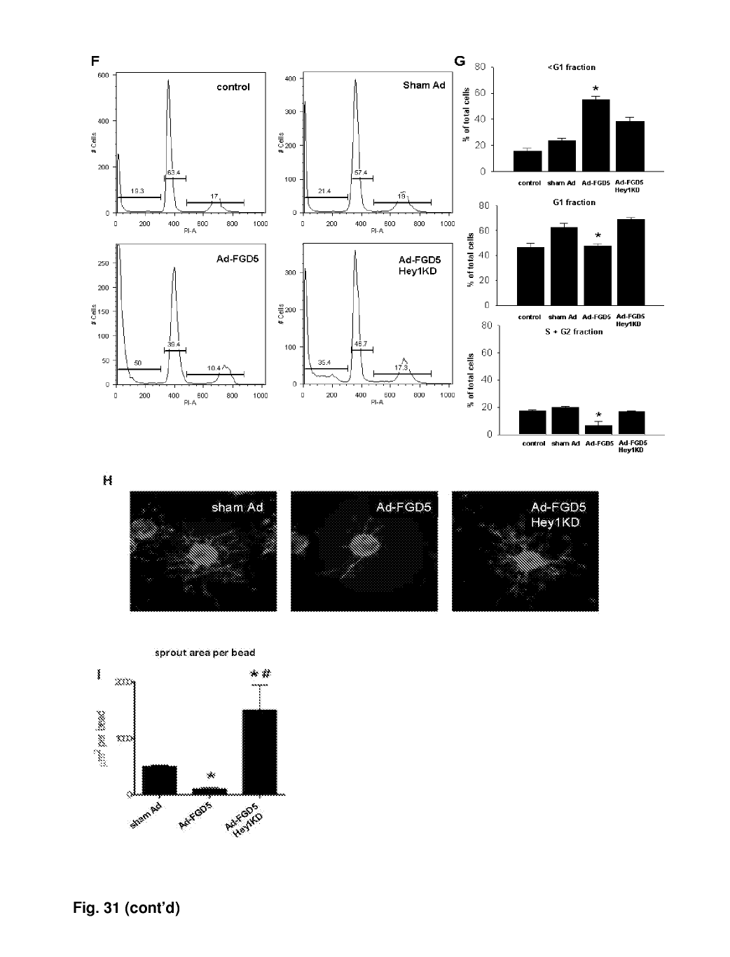

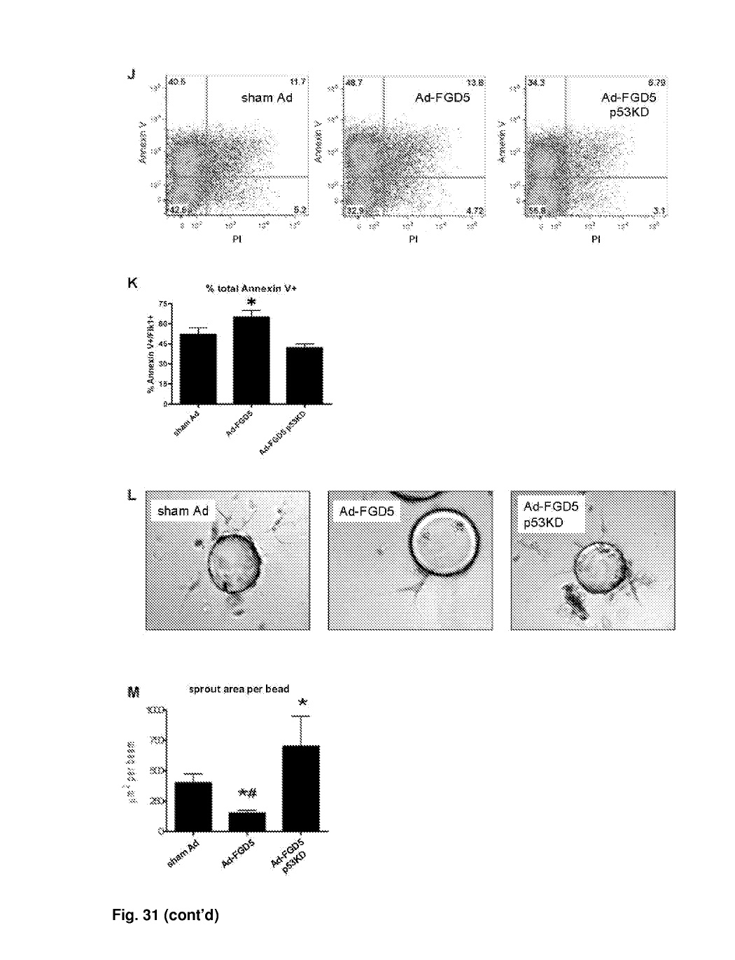

Hey1 overexpression has previously been shown to induce p53-mediated apoptosis in. This would suggest that Hey1 could mediate the FGD5-induced apoptotic response in ECs. Hey1 knockdown in FGD5-expressing HUVECs normalized growth inhibition by FGD5 (FIG. 31A), diminished p53 and p21CIP1 upregulation (FIG. 31B, C), and rescued ECs from apoptosis induced by FGD5 (FIG. 31D, E), whereas co-transfection with scrambled siRNA had no effect. Propidium Iodide-aided assessment using flow cytometric analysis suggested that FGD5 promoted the sub-G1 apoptotic fraction (FIG. 31 F, G), while the S+G2/G1 ratio decreased (20/64=0.31 compared with 8/45=0.17 for sham and FGD5 virus transduced HUVECs respectively), suggesting a Golgi cell cycle arrest (FIG. 31 F,G). This cytostatic effect was rescued by Hey1 knockdown in FGD5 overexpressing ECs (FIG. 31 F,G). Likewise, in vitro coated-bead assay demonstrated that FGD5 inhibition of capillary outgrowth could also be rescued by concomitant Hey1 silencing (FIG. 31 H, I).

FGD5-induced apoptosis was mediated via p53, as co-transfection of FGD5 overexpressing HUVECs with p53-targeting siRNA failed to induce apoptosis in ECs, as demonstrated by flow cytometry (FIG. 31 J, K), and reversed the growth-inhibitory effects of FGD5 overexpression in the coated bead assay (FIG. 31 L, M). These data support a pro-apoptotic function for FGD5 during late neo-angiogenesis and show that Hey1-p53 signalling cascade is crucial in the induction of FGD5-mediated programmed cell death in ECs.

FGD5 Binds and Activates cdc42.

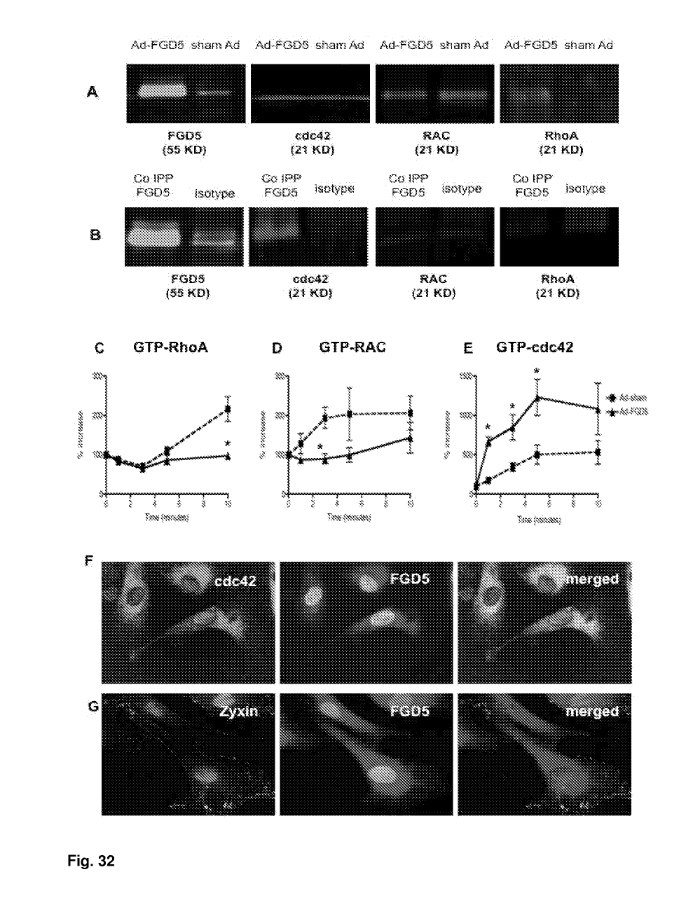

To determine the direct protein target of FGD5, we assessed whether the Dbl-homology (DH) domain and PH domain of FGD5 could bind and activate small GTPases. FGD5-transgene expression in HUVECs did not affect total cdc42, Rac1, or RhoA protein levels as compared to sham controls (FIG. 32 A). Co-immunoprecipitation (IP) using an antibody against FGD5 in protein lysates of FGD5 overexpressing HUVECs identified cdc42 as the selective protein-binding partner of FGD5 (FIG. 32 B), whereas cdc42-FGD5 binding led to specific activation of cdc42 in a GEF-activity assay (FIG. 32 C-E). In addition, fluorescent microscopy showed that endogenous FGD5 co-localized with cdc42 at the perinuclear site in the cytoplasm of ECs (FIG. 32 F, G). Previous studies have demonstrated cdc42 activation by FGD1, FGD4 and FRG, by conversion into GTP-bound cdc42 in cultured fibroblasts. Similarly, co-localization of FGD1 and cdc42 was observed in the Golgi-complex in Hela cells. Although this preservation of cdc42 GEF activity by the different FGD members seemed to imply a redundancy in the protein family by overlapping function, the specific expression of FGD5 in ECs point towards an unique role for this particular FGD-member in vessel formation.

Previous studies showed an increase in systemic cdc42 activity during natural aging. Constitutively elevated cdc42-GTP levels in a transgenic mouse line led to premature aging with failing DNA repair, accelerated cell senescence, and increased p53-dependent apoptosis. Similarly, cdc42 activation in cell culture was previously associated with cell senescence, and apoptosis, whereas cdc42 deletion in hematopoietic stem cells promoted proliferation by loss of p21CIP1 regulation of cell cycle progression. Our data confirm the direct link between cdc42 activity and p53-mediated apoptosis, as FGD5-mediated cdc42 activation into cdc42-GTP in ECs, leading to Hey1-mediated p53-dependent cell death. During vascular development, the vascular plexus is reorganized by vascular pruning, a process mainly mediated by active EC-apoptosis to remove aberrant neo capillaries. Previously, a number of genes including EphB4/ephrinB2, FasL-Fas, thrombospondin 1, angiostatin, and endostatin have been identified to show an inhibitory function in angiogenesis by providing a pro-apoptotic stimulus to ECs. In the light of the remodelling of the vascular network, FGD5 could play an important EC-selective role in this particular process, contributing to vascular pruning of redundant neovessels by induction of targeted apoptosis.

1. A method for modulating neovascularisation of a tissue in a subject in need thereof, said method comprising administering to said subject a therapeutically effective amount of a compound or a combination of compounds selected from:
an isolated nucleic acid molecule comprising a gene selected from the group consisting of RIKEN cDNA 9430020K01, Agtrl1, Apelin, Stabilin 1, Stabilin 2, TNFaip8l1, TNFaip8 and FGD5, and homologues thereof;
a gene product encoded by a gene selected from the group consisting of RIKEN cDNA 9430020K01, Agtrl1, Apelin, Stabilin 1, Stabilin 2, TNFaip8l1, TNFaip8, and FGD5, or encoded by homologues of these genes, and functional fragments thereof;
an antibody or derivative thereof directed against a gene product of a gene selected from the group consisting of RIKEN cDNA 9430020K01, Agtrl1, Apelin, Stabilin 1, Stabilin 2, TNFaip8l1, TNFaip8, and FGD5, or encoded by homologues of these genes, and functional fragments thereof, said derivative preferably being selected from the group consisting of scFv fragments, Fab fragments, chimeric antibodies, bifunctional antibodies, intrabodies, and other antibody-derived molecules;
an antisense molecule, in particular an antisense RNA or antisense oligodeoxynucleotide, an RNAi molecule (siRNA or miRNA) or a ribozyme capable of binding under stringent hybridization conditions to a gene or an mRNA gene product of the genes selected from the group consisting of RIKEN cDNA 9430020K01, Agtrl1, Apelin, Stabilin 1, Stabilin 2, TNFaip8l1, TNFaip8, and FGD5 and homologues thereof;
a small molecule interfering with the biological activity of a gene product of a gene selected from the group consisting of RIKEN cDNA 9430020K01, Agtrl1, Apelin, Stabilin 1, Stabilin 2, TNFaip8l1, TNFaip8, and FGD5 and homologues thereof, and
a (glycol)protein, a hormone and other biologically active compounds capable of interacting with a gene selected from the group consisting of RIKEN cDNA 9430020K01, Agtrl1, Apelin, Stabilin 1, Stabilin 2, TNFaip8l1, TNFaip8, and FGD5 and homologues thereof or with a gene product thereof, wherein said gene homologues have at least 60% sequence identity with the sequence of said gene.

2. Method according to claim 1, wherein said method for modulating neovascularisation is a method for:
treating or alleviating or preventing the risk of suffering from cardiovascular disease (including cardiovascular and cerebrovascular ischemic disease and peripheral artery disease), improving arterial healing following physical damage (stenting, medical intervention), treating or alleviating or preventing atherosclerosis, treating or alleviating or preventing atherosclerotic plaque formation, treating or alleviating or preventing plaque destabilization (vulnerable plaque formation and rupture); treating or alleviating or preventing cancer, in particular tumor angiogenesis; treating or alleviating or preventing diabetic retinopathy or retina retinopathy or any condition associated with enhanced, aberrant, immature, accelerated and/or uncoordinated vessel growth resulting in leaky or hyperpermeable vessels;
treatment to induce arterial remodeling and arterial integrity and/or hyperpermeability;
treatment to stimulate re-endothelialisation of compounds, grafts and/or devices (valves, vascular grafts, endovascular prosthesis, intravascular stents) to reduce the risk of thrombus formation thereon.

3. Pharmaceutical composition comprising a therapeutically effective amount of at least one compound as defined in claim 1 and a pharmaceutically acceptable excipient, carrier or diluent.

4. Pharmaceutical composition according to claim 3, wherein said compound is
an isolated nucleic acid molecule comprising a gene selected from the group consisting of RIKEN cDNA 9430020K01 and homologues thereof;
a gene product encoded by the gene RIKEN cDNA 9430020K01 or encoded by homologues of this gene, and functional fragments thereof;
an antibody or derivative thereof directed against a gene product of the gene RIKEN cDNA 9430020K01 or encoded by homologues of this gene, and functional fragments thereof, said derivative preferably being selected from the group consisting of scFv fragments, Fab fragments, chimeric antibodies, bifunctional antibodies, intrabodies, and other antibody-derived molecules;
an antisense molecule, in particular an antisense RNA or antisense oligodeoxynucleotide, an RNAi molecule (siRNA or miRNA) or a ribozyme capable of binding under stringent hybridization conditions to a gene or an mRNA gene product of the genes selected from the group consisting of RIKEN cDNA 9430020K01 and homologues thereof;
a small molecule interfering with the biological activity of a gene product of a gene selected from the group consisting of RIKEN cDNA 9430020K01 and homologues thereof, and
a (glycol)protein, a hormone and other biologically active compounds capable of interacting with a gene or gene product selected from the group consisting of RIKEN cDNA 9430020K01 and homologues thereof,
wherein said gene homologues have at least 60% sequence identity with the sequence of said gene.

5. Method of treating a subject, comprising administering to said subject a therapeutically effective amount of the pharmaceutical composition of claim 3.

6. Method according to claim 5, wherein said treatment comprises:
the treatment or alleviation or prevention of the risk of suffering from cardiovascular disease, improving arterial healing following physical damage (stenting, medical intervention), treatment or alleviation or prevention of atherosclerosis, treatment or alleviation or prevention of atherosclerotic plaque formation, treatment or alleviation or prevention of plaque destabilization (vulnerable plaque formation and rupture); treatment or alleviation or prevention of cancer, in particular tumor angiogenesis; treatment or alleviation or prevention of diabetic retinopathy or retina retinopathy or any condition associated with enhanced, aberrant, immature, accelerated and/or uncoordinated vessel growth resulting in leaky or hyperpermeable vessels;
the treatment to induce arterial remodeling and arterial integrity/hyperpermeability;
the treatment to stimulate re-endothelialisation of compounds, grafts and/or devices (valves, vascular grafts, endovascular prosthesis, intravascular stents) to reduce the risk of thrombus formation thereon.

7. Compound selected from:
an isolated nucleic acid molecule comprising a gene selected from the group consisting of RIKEN cDNA 9430020K01, Agtrl1, Apelin, Stabilin 1, Stabilin 2, TNFaip8l1, TNFaip8 and FGD5, and homologues thereof;
a gene product encoded by a gene selected from the group consisting of RIKEN cDNA 9430020K01, Agtrl1, Apelin, Stabilin 1, Stabilin 2, TNFaip8l1, TNFaip8, and FGD5, or encoded by homologues of these genes, and functional fragments thereof;
an antibody or derivative thereof directed against a gene product of a gene selected from the group consisting of RIKEN cDNA 9430020K01, Agtrl1, Apelin, Stabilin 1, Stabilin 2, TNFaip8l1, TNFaip8, and FGD5, or encoded by homologues of these genes, and functional fragments thereof, said derivative preferably being selected from the group consisting of scFv fragments, Fab fragments, chimeric antibodies, bifunctional antibodies, intrabodies, and other antibody-derived molecules;
an antisense molecule, in particular an antisense RNA or antisense oligodeoxynucleotide, an RNAi molecule (siRNA or miRNA) or a ribozyme capable of binding under stringent hybridization conditions to a gene or an mRNA gene product of the genes selected from the group consisting of RIKEN cDNA 9430020K01, Agtrl1, Apelin, Stabilin 1, Stabilin 2, TNFaip8l1, TNFaip8, and FGD5 and homologues thereof;
a small molecule interfering with the biological activity of a gene product of a gene selected from the group consisting of RIKEN cDNA 9430020K01, Agtrl1, Apelin, Stabilin 1, Stabilin 2, TNFaip8l1, TNFaip8, and FGD5 and homologues thereof, and
a (glycol)protein, a hormone and other biologically active compounds capable of interacting with a gene selected from the group consisting of RIKEN cDNA 9430020K01, Agtrl1, Apelin, Stabilin 1, Stabilin 2, TNFaip8l1, TNFaip8, and FGD5 and homologues thereof or with a gene product thereof, wherein said gene homologues have at least 60% sequence identity with the sequence of said gene, for use as a medicament in:
the treatment or alleviation or prevention of the risk of suffering from cardiovascular disease, improving arterial healing following physical damage (stenting, medical intervention), treatment or alleviation or prevention of atherosclerosis, treatment or alleviation or prevention of atherosclerotic plaque formation, treatment or alleviation or prevention of plaque destabilization (vulnerable plaque formation and rupture); treatment or alleviation or prevention of cancer, in particular tumor angiogenesis; treatment or alleviation or prevention of diabetic retinopathy or retina retinopathy or any condition associated with enhanced, aberrant, immature, accelerated and/or uncoordinated vessel growth resulting in leaky or hyperpermeable vessels;
the treatment to induce arterial remodeling and arterial integrity/hyperpermeability;
the treatment to stimulate re-endothelialisation of compounds, grafts and/or devices (valves, vascular grafts, endovascular prosthesis, intravascular stents) to reduce the risk of thrombus formation thereon.

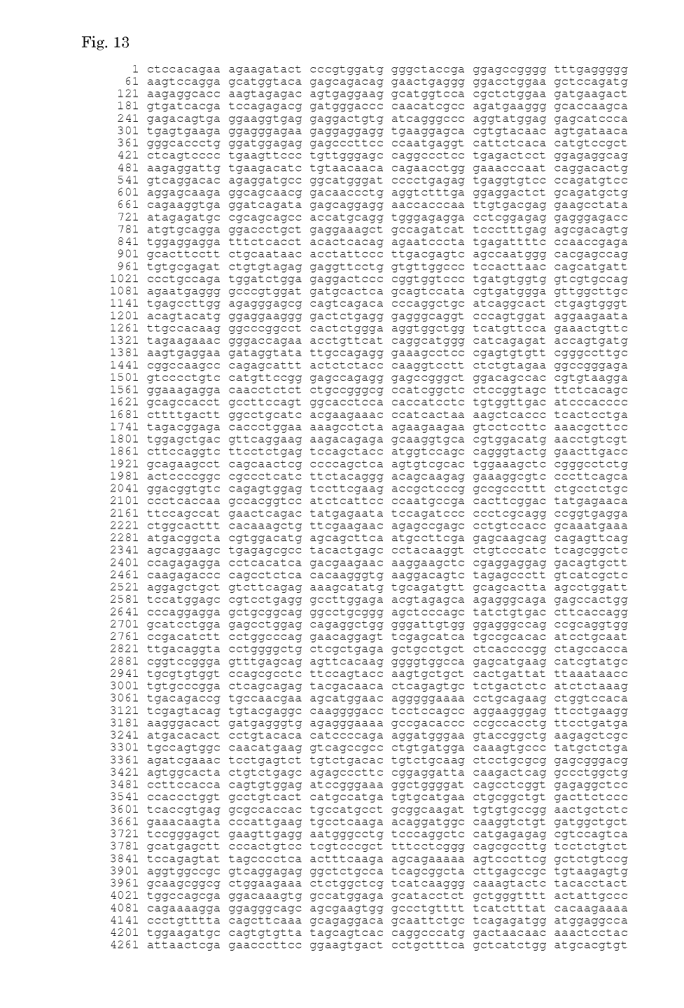

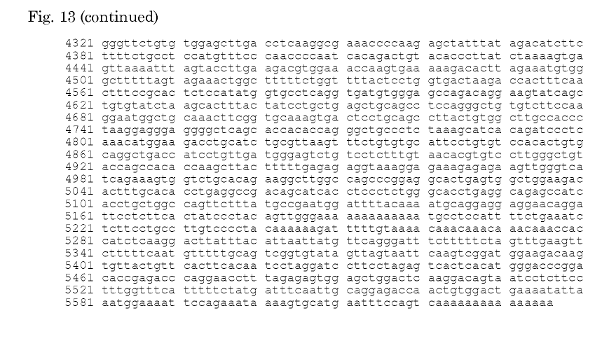

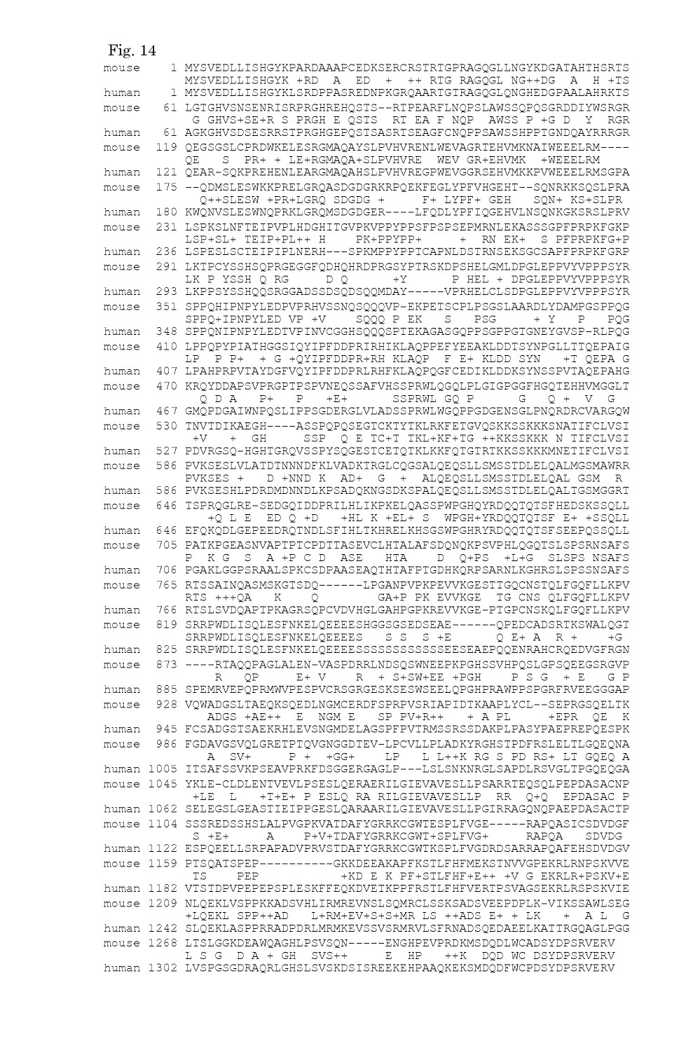

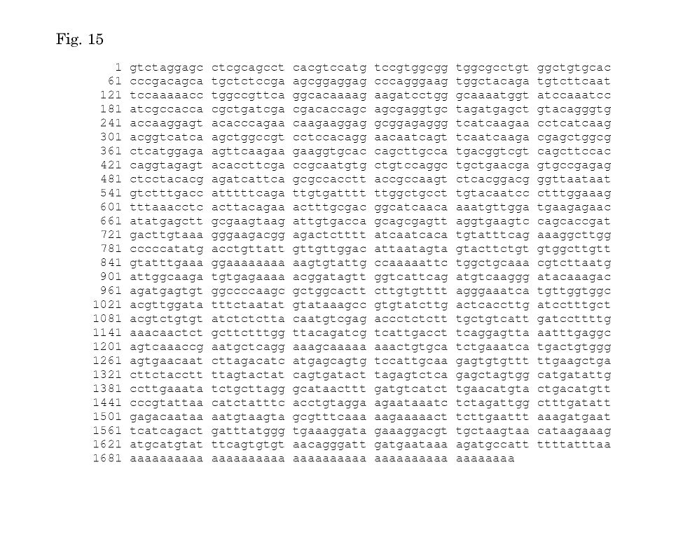

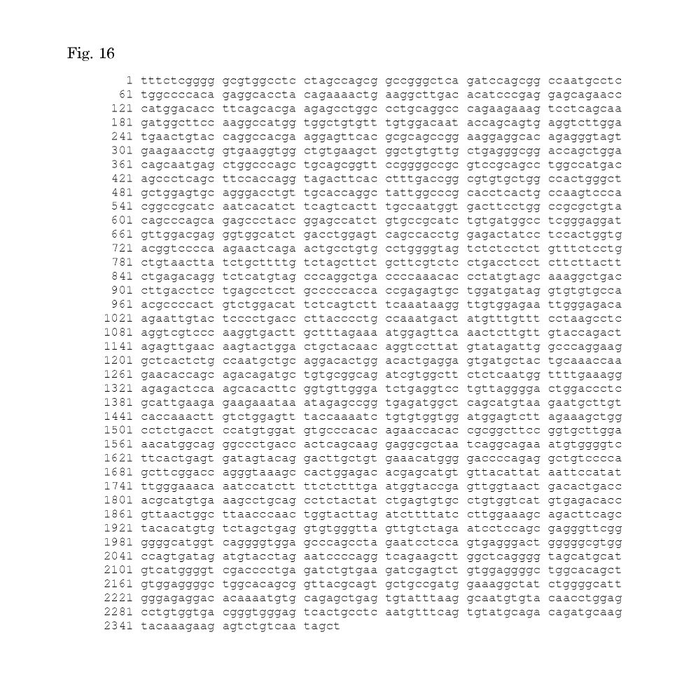

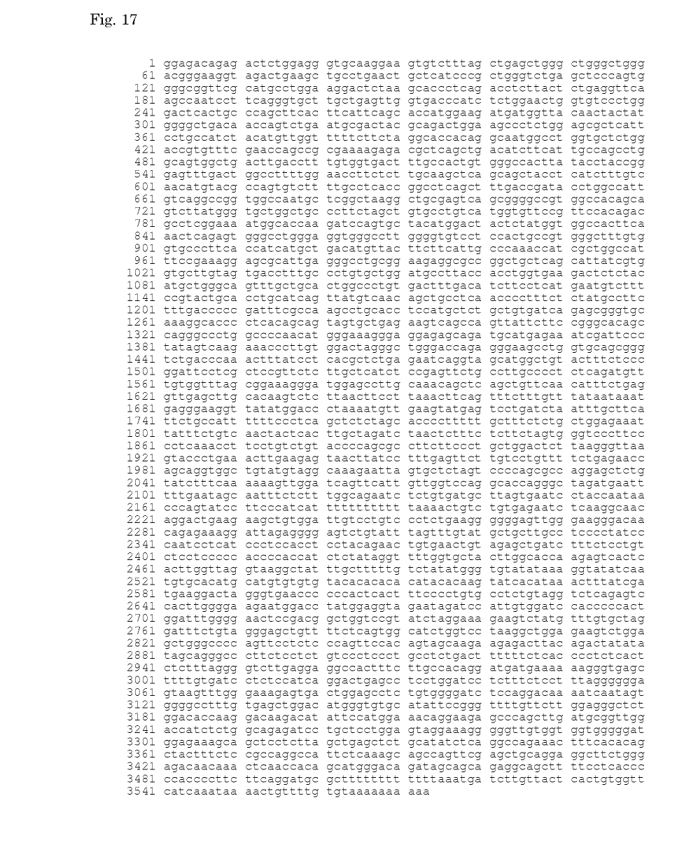

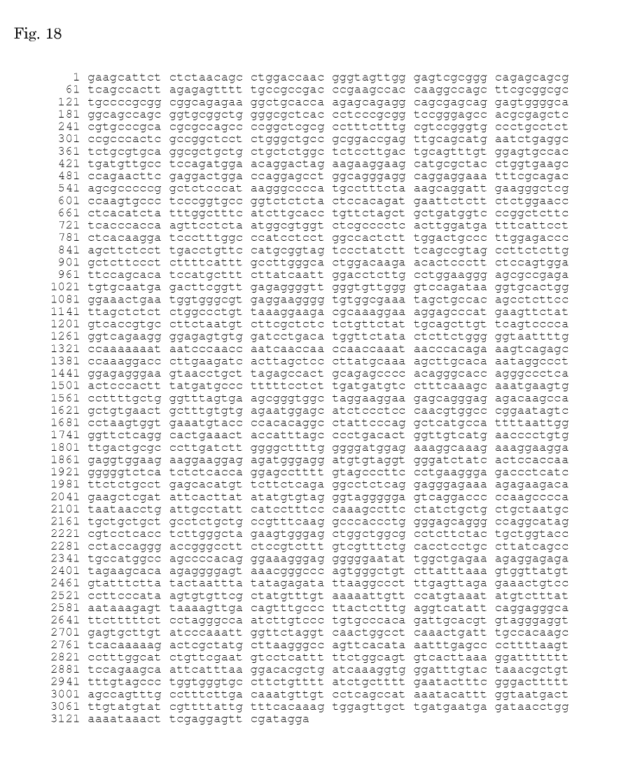

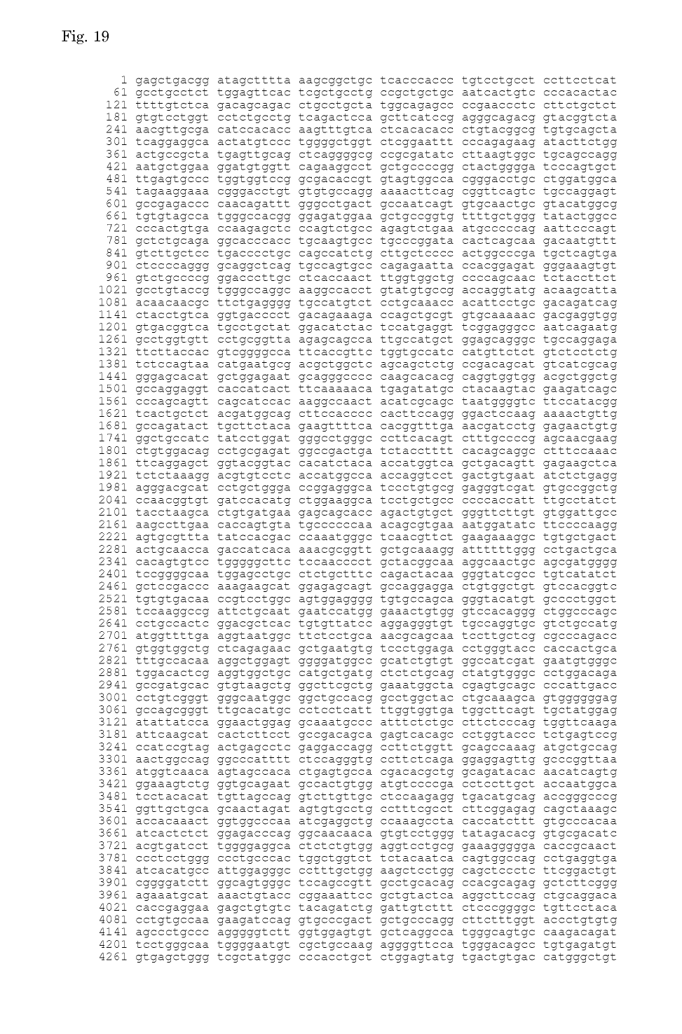

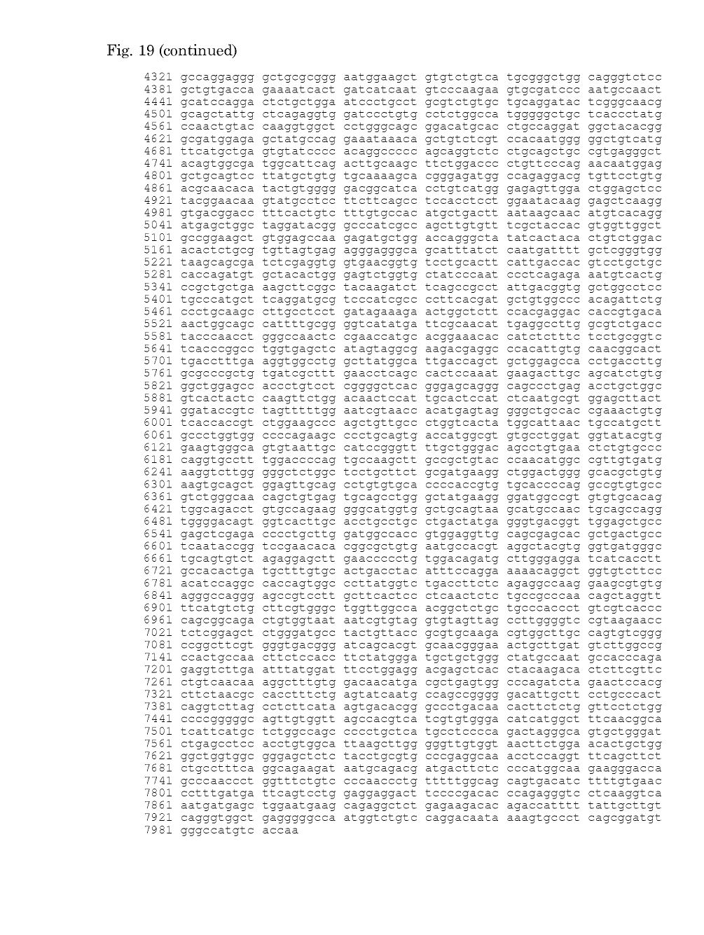

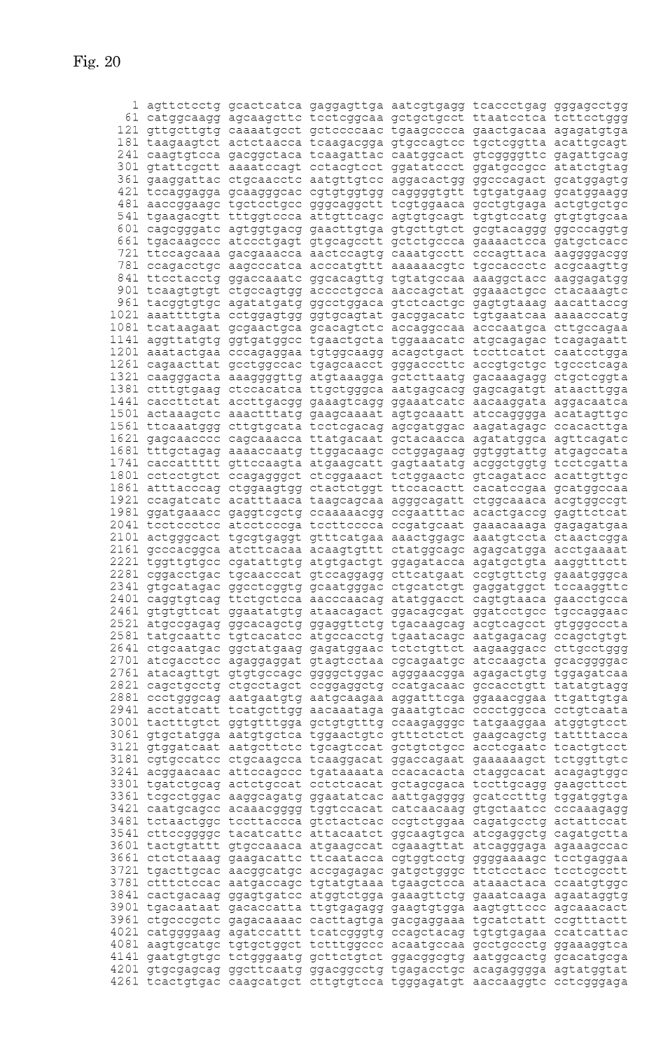

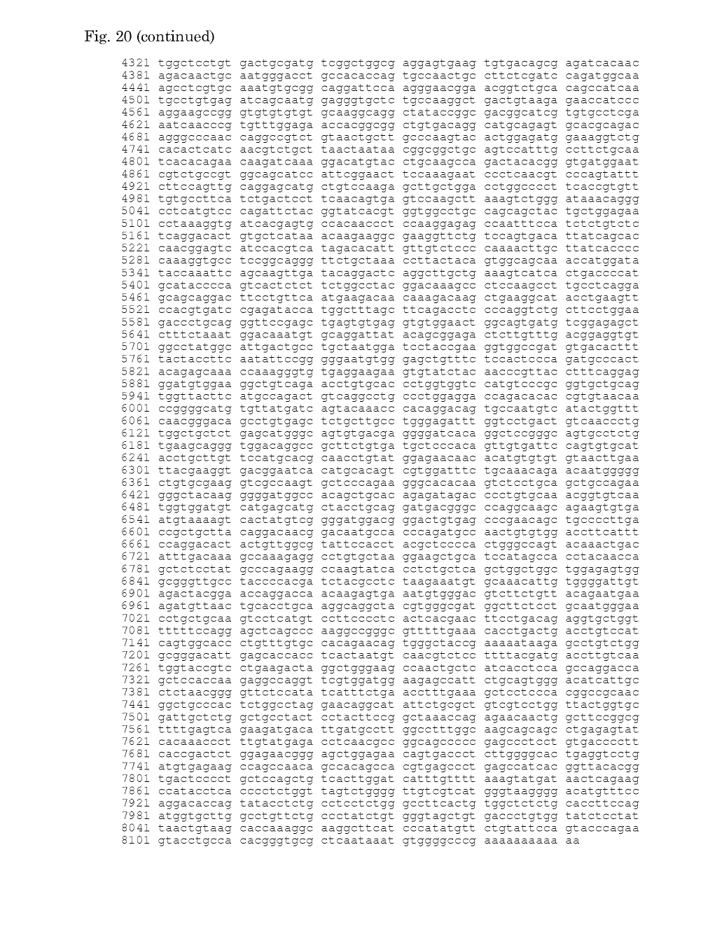

8. An isolated nucleic acid molecule comprising a sequence which has a sequence identity of at least 60% with the sequence as indicated in any one of FIGS. 11, 13, and 15-20.

9. A gene product of an isolated nucleic acid molecule according to claim 8 or a vector comprising the nucleic acid molecule according to claim 8 for use as a medicament.

10. A gene product or a vector according to claim 9 for use in:
the treatment or alleviation or prevention of the risk of suffering from cardiovascular disease, improving arterial healing following physical damage (stenting, medical intervention), treatment or alleviation or prevention of atherosclerosis, treatment or alleviation or prevention of atherosclerotic plaque formation, treatment or alleviation or prevention of plaque destabilization (vulnerable plaque formation and rupture); treatment or alleviation or prevention of cancer, in particular tumor angiogenesis; treatment or alleviation or prevention of diabetic retinopathy or retina retinopathy or any condition associated with enhanced, aberrant, immature, accelerated and/or uncoordinated vessel growth resulting in leaky or hyperpermeable vessels;
the treatment to induce arterial remodeling and arterial integrity/hyperpermeability;
the treatment to stimulate re-endothelialisation of compounds, grafts and/or devices (valves, vascular grafts, endovascular prosthesis, intravascular stents) to reduce the risk of thrombus formation thereon.

11. Method of treating a subject, comprising administering to said subject a therapeutically effective amount of the pharmaceutical composition of claim 4.

12. Method according to claim 11, wherein said treatment comprises:
the treatment or alleviation or prevention of the risk of suffering from cardiovascular disease, improving arterial healing following physical damage (stenting, medical intervention), treatment or alleviation or prevention of atherosclerosis, treatment or alleviation or prevention of atherosclerotic plaque formation, treatment or alleviation or prevention of plaque destabilization (vulnerable plaque formation and rupture); treatment or alleviation or prevention of cancer, in particular tumor angiogenesis; treatment or alleviation or prevention of diabetic retinopathy or retina retinopathy or any condition associated with enhanced, aberrant, immature, accelerated and/or uncoordinated vessel growth resulting in leaky or hyperpermeable vessels;
the treatment to induce arterial remodeling and arterial integrity/hyperpermeability;
the treatment to stimulate re-endothelialisation of compounds, grafts and/or devices (valves, vascular grafts, endovascular prosthesis, intravascular stents) to reduce the risk of thrombus formation thereon.
